(19) United States
(12) Patent Application Publication
(10) Pub. No.: US 2022/0315661 A1
(43) Pub. Date: Oct. 6, 2022

(54) DOSAGE REGIMENS FOR A COMBINATION OF ANTI-DR5 ANTIBODIES FOR USE IN TREATING CANCER

(71) Applicant: GENMAB B.V., Utrecht (NL)

(72) Inventors: Ulf FORSSMANN, Hannover (DE); Manish GUPTA, Skillman, NJ (US); Jens Thing MORTENSEN, Ballerup (DK); Merete ELLEKILDE-PEDERSEN, Vaerloese (DK); Marije Berber OVERDIJK, Utrecht (NL); Tahamtan AHMADI, Rydal, PA (US)

(21) Appl. No.: 17/609,359

(22) PCT Filed: May 11, 2020
(86) PCT No.: PCT/EP2020/063101

§ 371 Date: Nov. 5, 2021

Related U.S. Application Data
(60) Provisional application No. 62/845,746, filed on May 9, 2019.

(51) Int. Cl. C07K 16/28; A61P 35/00; A61K 39/395; A61K 31/573; A61P 35/02

(52) U.S. Cl. CPC C07K 16/2878; A61P 35/00; A61K 39/39558; A61K 31/573; A61P 35/02; A61K 2039/545

(57) ABSTRACT

The present invention relates to a combination of two antibody molecules that bind to human DR5 antigen and their use in treating cancer. In particular, the present invention relates to dosage regimens for such anti-DR5 antibodies comprising administering to subject weekly dosages followed by biweekly dosages, or one or two priming dosage(s) followed by biweekly dosages.

### FIELD OF THE INVENTION

The present invention relates, inter alia, to a combination of two antibody molecules that bind to human DR5 antigen and their use in treating cancer. In particular, the present invention relates to dosage regimens for such anti-DR5 antibodies comprising administering to subject weekly dosages followed by biweekly dosages, or one or two priming dosage(s) followed by biweekly dosages.

### BACKGROUND OF THE INVENTION

DR5, also known as death receptor 5, Tumor necrosis factor receptor superfamily member 10B, TNFRSF10B, TNF-related apoptosis-inducing ligand receptor 2, TRAIL receptor 2, TRAIL-R2 and CD262, is a cell surface receptor of the TNF receptor superfamily that binds tumor necrosis factor-related apoptosis-inducing ligand (TRAIL) and mediates apoptosis. In the absence of ligand, DR5 exists in the cell membrane either as monomer or as pre-assembled complexes of two or three receptors through interactions of the first cysteine-rich domain, also known as pre-ligand assembly domain (PLAD). A Crystal structure of TRAIL in complex with the DR5 ectodomain showed that TRAIL binds to DR5 in a complex containing a trimeric receptor and a trimeric ligand (Hymowitz et al., Mol Cell. 1999 October; 4(4):563-71). In the ligand-bound conformation, the cytoplasmic FAS-adaptor protein with a death domain (FADD) associate with the intracellular DD surface of the oligomerized DR5 molecules and engage initiator caspases caspase-8 and caspase-10 to form the death-inducing signaling complex (DISC).

Based on the sensitivity of cancer cells to TRAIL-mediated apoptosis, numerous agents were developed to activate this pathway to induce apoptosis selectively in cancer cells. A series of conventional (monospecific, bivalent) anti-DR5 antibodies have been developed and tested in the clinic (reviewed in Ashkenazi et al., Nat Rev Drug Discov. 2008 December; 7(12):1001-12; Trivedi et al., Front Oncol. 2015 Apr. 2; 5:69; Yuan et al., Cancer Metastasis Rev 2018 December; 37(4):733-748). Clinical studies with these compounds demonstrated that DR5 antibodies were generally well tolerated but failed to show convincing and significant clinical benefit.

A combination of two non-competing anti-DR5 antibodies comprising an Fc region of a human IgG1 and an antigen binding region binding to DR5, wherein the Fc region comprises an E430G mutation, was found to facilitate hexamerization of the antibodies on the cell-surface upon antigen binding and significantly enhances the potency of the antibodies in inducing apoptosis and cell death Accordingly, there remains an unmet medical need for patients suffering from solid tumors and hematological malignancies and anti-DR5 antibodies offer a promising strategy. However, there is a need for providing improved dosage regimens for the antibodies described in PCT/EP2016/079518.

### OBJECT OF THE INVENTION

It is an object of the present invention to provide methods for treating solid tumors and hematological malignancies. It is a further object of the present invention to provide a dosage regimen for a combination of a first and a second antibody binding to DR5 for use in methods of treating such cancers. It is a further object of the present invention to provide a safe and efficacious new dosage regimen for a first and a second antibody binding to DR5.

### SUMMARY OF THE INVENTION

The present inventors have developed an improved dosage regimen of a biweekly dosage regimen for a combination of a first anti-DR5 antibody and a second anti-DR5 antibody, which provides an efficacious therapeutic regimen and has acceptable tolerability and safety profiles. Accordingly, the present invention relates to a first and second anti-DR5 antibody for use in the treatment of solid cancers or hematological malignancies wherein the first and second anti-DR5 antibody is administered once a week for an eight-weeks period followed by a biweekly dosage, or twice in a two-weeks period followed by a biweekly dosage, or on day one of a first and second two-weeks period followed by a biweekly dosage. Thus, in one aspect, the invention relates to a method of treating a solid tumor or a hematological malignancy in a subject, the method comprising administering to a subject in need thereof a first antibody that binds DR5 and a second antibody that binds DR5, or a pharmaceutically acceptable salt thereof, wherein the first antibody and the second antibody is administered on: i) day 1 and day 8 of a 14-days cycle for the first four cycles (intensified; or ii) day 1 and day 8 of a first 14-days cycle (priming); or iii) day 1 of a first 14-day cycle (priming); iv) day 1 of a first and second 14-days cycle (priming); followed by administration on day 1 of a 14-days cycle (Q2W).

In a further aspect the invention relates to a first and second antibody that binds DR5, or a pharmaceutically acceptable salt thereof, for use in the treatment of a solid tumor or a hematological malignancy, wherein the first antibody and the second antibody, or pharmaceutically acceptable salt thereof, is administered on, i) day 1 and day 8 of a 14-days cycle for the first four cycles; or ii) day 1 and day 8 of a first 14-days cycle (priming); or iii)) day 1 of a first 14-day cycle (priming); iv) day 1 of a first and second 14-days cycle (priming); followed by administration on day 1 of a 14-days cycle (Q2W). In one embodiment the first dose is a priming dose or the first and second dose is a priming dose. The priming dose is a lower dose in the rage of about 0.05 mg/kg-0.15 mg/kg of each first and second antibody. Thus, the combined dose of the first and second antibody in the priming dose is in the range of about 0.1 mg/kg to 0.3 mg/kg. The priming dose is a lower dose that the treatment dose administered to an individual biweekly (Q2W) following a single priming dose or two priming doses. In a preferred embodiment the priming dose of each first and second antibody is 0.05 mg/kg.

In a preferred embodiment the priming dose of the combined first and second antibody is 0.1 mg/kg. Following the priming treatment dose, a subject in need of treatment may be administered further treatment doses based on a biweekly dosing schedule (on day 1 of a 14 days-cycle (Q2W)). Treatment doses following the priming dose may be in the range of 0.3 mg/kg to 9 mg/kg for each antibody. In one embodiment of the invention the treatment dose administered on day 1 of a 14-day cycle is in the range of 0.15 mg/kg to 9 mg/kg for each of the first and second antibody. In one embodiment of the invention the treatment dose administered on day 1 of a 14-day cycle is in the range of 0.15 mg/kg to 9 mg/kg for the for each of the first and second antibody. In a preferred embodiment the treatment dose administered on day 1 of a 14-day cycle is in the range of 0.15 mg/kg to 3 mg/kg for the for each of the first and second antibody. In one embodiment of the invention the combined treatment dose of the first and second antibody administered on day 1 of a 14-day cycle is within the range of 0.3 mg/kg to 6 mg/kg.

### BRIEF DESCRIPTION OF THE DRAWINGS

FIG. 1: Binding to CHO-S expressing human and cynomolgus monkey DR5. Antibody binding was tested by flow cytometry using CHO-S cells expressing (A) human DR5 and (B) cynomolgus monkey DR5. IgG1-b12 was used as an isotype control antibody. Binding is expressed as the geometric mean of the fluorescence intensity of duplicate samples ±SD.

FIG. 2: Binding of DR5-specific antibodies to HCT 116 cells. Binding of IgG1-hDR5-01-G56T-E430G and IgG1-hDR5-05-E430G to HCT 116 cells was measured by flow cytometry, and compared to the WT antibodies without the hexamerization-enhancing mutation E430G. IgG1-b12 was used as an isotype control antibody. The graph shows the Geomean fluorescence of duplicate measurements ±standard deviation (SD) of a representative experiment.

FIG. 3: Mapping of binding regions using domain-swapped DR5 molecules. (A) Sequence alignment of part of the extracellular domains of human DR5 and mouse DR5 using EMBOSS Matcher (http://www.ebi.ac.uk/Tools/psa/emboss_matcher/); (.) similar amino acid; (:) identical amino acid. (B) Graphical representation of the domain-swapped DR5 extracellular domain (white: human DR5 sequences; black: mouse DR5 sequences). Amino acid number refer to the human sequence and domain swaps were made based on the alignment shown in panel A. (C) Binding of IgG1-hDR5-01-F405L and the isotype control antibody IgG1-b12 to a panel of human-mouse chimeric DR5 molecules, as assessed by flow cytometry. In each domain-swapped DR5 molecule, specific human amino acids have been replaced by the mouse sequence, as indicated on the X-axis. Error bars indicate the standard deviation of duplicate samples. (D) Binding of IgG1-hDR5-05-F405L to a panel of human-mouse chimeric DR5 molecules, as assessed by flow cytometry. In each domain-swapped DR5 molecule, specific human amino acids had been replaced by the mouse sequence, as indicated on the X-axis. IgG1-b12 was included an isotype control antibody. Error bars indicate the standard deviation of duplicate samples.

FIG. 4: Antibody-specific binding ELISA. Differential binding of IgG1-hDR5-01-G56T-E430G and IgG1-hDR5-05-E430G to recombinant DR5 variants was investigated in a binding ELISA. IgG1-b12 was used as an isotype control antibody. Data show concentration-dependent binding to immobilized DR5 variants, as measured by OD at 405 nm. (A) Binding to DR5ECD-FcRbHisCtag; (B) Binding to DR5sh79-115ECDdel-FcRbHisCtag; (C) Binding to DR5sh139-166ECDdel-FcRbHisCtag.

FIG. 5: Association and dissociation curves of soluble recombinant extracellular domain of human DR5 binding to DR5-specific antibodies. The affinities for binding to kDR5ECDdelHis were measured for IgG1-hDR5-01-G56T-E430G and IgG1-hDR5-05-E430G and compared to the WT IgG1 antibodies using Bio-Layer Interferometry on a ForteBio Octet HTX. Data show for each antibody the association and dissociation traces (in black) and the fit (in red) of a representative experiment. (A) IgG1-hDR5-01-G56T; (B) IgG1-hDR5-01-G56T-E430G; (C) IgG1-hDR5-05; (D) IgG1-hDR5-05-E430G.

FIG. 6: Viability assay in vitro for the mixture of IgG1-hDR5-01-G56T-E430G and IgG1-hDR5-05-E430G using cell lines. A three-day viability assay was performed with several cell lines to test the cytotoxicity of the mixture of IgG1-hDR5-01-G56T-E430G and IgG1-hDR5-05-E430G. IgG1-b12 was used as an isotype control antibody. Cell viability was determined using the CellTiter-Glo kit. Data shown is the mean of duplicate samples ±standard deviation (SD).

FIG. 7: Cytotoxicity screening in a broad range of cell lines representing solid tumor indications. The cytotoxic capacity of the mixture of IgG1-hDR5-01-G56T-E430G and IgG1-hDR5-05-E430G was explored in viability assays using a panel of 240 cell lines representing 16 solid cancer lineages. Cell viability was determined using the ATPlite kit. (A) Percentage viable cells after incubation with the mixture of IgG1-hDR5-01-G56T-E430G and IgG1-hDR5-05-E430G, each data point represents an individual cell line of the indicated human cancer type; horizontal solid lines represent the mean for each human cancer type. Cell lines that showed ≤30% viable cells after antibody treatment (indicated by the dotted line) were classified as responders. (B) The percentage responding cell lines within each lineage. Cancer types for which more than 5 cell lines were tested are included in the graph (number of cell lines tested in parenthesis).

FIG. 8: Cytotoxicity screening in cell lines representing different hematological malignancies. The cytotoxic capacity of the mixture of IgG1-hDR5-01-G56T-E430G and IgG1-hDR5-05-E430G was explored in viability assays using a panel of 45 cell lines representing 5 hematological cancer lineages. Cell viability was determined using the CellTiter-Glo 2.0 proliferation assay. The dot plot presents percentage viable cells after incubation with the mixture of IgG1-hDR5-01-G56T-E430G and IgG1-hDR5-05-E430G, with each data point representing an individual cell line of the indicated human cancer type; horizontal solid lines represent the mean of the individual data points. DLBCL: diffuse large B-cell lymphoma; MCL: mantle cell lymphoma; AML: acute myeloid leukemia; MM: multiple myeloma.

FIG. 9: Dosage regimens in HCT-15 colorectal cancer CDX model. Different dosage regimens of the mixture of IgG1-hDR5-01-G56T-E430G and IgG1-hDR5-05-E430G were tested in a subcutaneous HCT-15 colorectal cancer xenograft, using IgG1-b12 antibody as isotype control. Mice were dosed Q7Dx3 with 0.5 mg/kg, Q7Dx2 with 0.75 mg/kg or 10 mg/kg or day 0, 3, 7, 10, 14, 21 with 0.25 mg/kg Hx-DR5-01/05. Tumor size (mean±standard error of mean [SEM]) in mice is shown in time.

FIG. 10: In vivo anti-tumor efficacy in COLO 205 colorectal cancer CDX model. Evaluation of the in vivo efficacy of different doses of the mixture of IgG1-DR5-01-K409R-E430G+IgG1-DR5-05-F405L-E430G in a subcutaneous COLO 205 colorectal cancer xenograft, using IgG1-b12 antibody as isotype control. Tumor size (mean±SEM) in mice treated with the indicated antibody dose is shown in time.

FIG. 11: In vivo anti-tumor efficacy in HCT-15 colorectal cancer CDX model. Evaluation of the in vivo efficacy of different doses of the mixture of IgG1-DR5-01-K409R-E430G+IgG1-DR5-05-F405L-E430G in a subcutaneous HCT-15 colorectal cancer xenograft, using IgG1-b12 antibody as isotype control. Tumor size (mean±SEM) in mice treated with the indicated antibody dose is shown in time.

FIG. 12: In vivo anti-tumor efficacy in SW480 colorectal cancer CDX model. Evaluation of the in vivo efficacy of different doses of the mixture of IgG1-DR5-01-K409R-E430G+IgG1-DR5-05-F405L-E430G in a subcutaneous SW480 colorectal cancer xenograft, using IgG1-b12 antibody as isotype control. Tumor size (mean±SEM) in mice treated with the indicated antibody dose is shown in time.

FIG. 13: In vivo anti-tumor efficacy in BxPC-3 pancreatic cancer CDX model. Evaluation of the in vivo efficacy of different doses of the mixture of IgG1-DR5-01-K409R-E430G+IgG1-DR5-05-F405L-E430G in a subcutaneous BxPC-3 pancreatic cancer xenograft, using IgG1-b12 antibody as isotype control. Tumor size (mean±SEM) in mice treated with the indicated antibody dose is shown in time.

FIG. 14: In vivo anti-tumor efficacy in PANC-1 pancreatic cancer CDX model. Evaluation of the in vivo efficacy of different doses of the mixture of IgG1-DR5-01-K409R-E430G+IgG1-DR5-05-F405L-E430G in a subcutaneous PANC-1 pancreatic cancer xenograft, using IgG1-b12 antibody as isotype control. Tumor size (mean±SEM) in mice treated with the indicated antibody dose is shown in time.

FIG. 15: In vivo anti-tumor efficacy in A375 melanoma CDX model. Evaluation of the in vivo efficacy of different doses of the mixture of IgG1-DR5-01-K409R-E430G+IgG1-DR5-05-F405L-E430G in a subcutaneous A375 melanoma xenograft, using IgG1-b12 antibody as isotype control. Tumor size (mean±SEM) in mice treated with the indicated antibody dose is shown in time.

FIG. 16: In vivo anti-tumor efficacy in SNU-5 gastric cancer CDX model. Evaluation of the in vivo efficacy of different doses of the mixture of IgG1-DR5-01-K409R-E430G+IgG1-DR5-05-F405L-E430G in a subcutaneous SNU-5 gastric cancer xenograft, using IgG1-b12 antibody as isotype control. Tumor size (mean±SEM) in mice treated with the indicated antibody dose is shown in time.

FIG. 17: In vivo anti-tumor efficacy in SK-MES-1 NSCLC CDX model. Evaluation of the in vivo efficacy of different doses of the mixture of IgG1-DR5-01-K409R-E430G+IgG1-DR5-05-F405L-E430G in a subcutaneous SK-MES-1 NSCLC xenograft, using IgG1-b12 antibody as isotype control. Tumor size (mean±SEM) in mice treated with the indicated antibody dose is shown in time.

FIG. 18: Efficacy in PDX clinical trials. The efficacy of 2 mg/kg of the mixture of IgG1-DR5-01-G56T-E430G+IgG1-DR5-05-E430G was tested in PDX models derived from colorectal cancer (n=70), NSCLC (n=62) and kidney cancer (n=5) using PBS as negative control. Responder, intermediates and non-responder models are defined by the relative tumor growth in the treated mouse versus the non-treated mouse (ΔT/ΔC value).

FIG. 19: Colorectal cancer PDX model CR0126. Evaluation of the in vivo efficacy of the mixture of IgG1-hDR5-01-G56T-E430G+IgG1-hDR5-05-E430G in colorectal cancer PDX model CR0126. IgG1-b12-E430G was used as negative control antibody (isotype control). Tumor size (mean±SEM) in mice treated with the indicated dose is shown in time.

FIG. 20: Colorectal cancer PDX model CR3056. Evaluation of the in vivo efficacy of the mixture of IgG1-hDR5-01-G56T-E430G+IgG1-hDR5-05-E430G in colorectal cancer PDX model CR3056. IgG1-b12-E430G was used as negative control antibody (isotype control). Tumor size (mean±SEM) in mice treated with the indicated dose is shown in time.

FIG. 21: Colorectal cancer PDX model CR3150. Evaluation of the in vivo efficacy of the mixture of IgG1-hDR5-01-G56T-E430G+IgG1-hDR5-05-E430G in colorectal cancer PDX model CR3150. IgG1-b12-E430G was used as negative control antibody (isotype control). Tumor size (mean±SEM) in mice treated with the indicated dose is shown in time.

FIG. 22: Total human IgG concentration in plasma from tumor-free immunodeficient CB17-SCID mice treated intravenously with 1 mg/kg IgG1-DR5-01-G56T-E430G or IgG1-DR5-05-E430G, or the mixture thereof. Total human IgG in plasma samples was determined by ELISA for each mouse using the mean of the four serial dilutions per sample and plotted in a concentration versus time curve. IgG1-b12 was used as an isotype control antibody. Each data point represents the mean±SEM from 3 individual mice.

FIG. 23: PK analysis in tumor-free mice. (A) Clearance (CL) rate until day 20 after administration of the antibody was determined following the formula (Dx1000)/AUC with D, injected dose and AUC, area under the curve of the concentration-time curve. (B) The peak plasma concentration (Cmax) as observed at 10 minutes after administration of the antibody. (C) Central volume of distribution (Vcen) was determined following the formula (Dose×1,000)/Cmax. IgG1-b12 was used as an isotype control antibody. Shown is the mean±SEM for the three mice per group.

FIG. 24: Plasma concentration-time profiles following a single i.v. dose of IgG1-hDR5-05-E430G+IgG1-hDR5-01-G56T-E430G in female cynomolgus monkeys. Three dose levels were tested with three female monkeys each: (A) 0.5 mg/kg; (B) 5 mg/kg; (C) 25 mg/kg. Post-dose samples were taken at 1, 3, 6, 12, 24 hours, 2, 3, 7, 14, 21, 22, 35, 49 days after dosing. The dotted line indicates the predicted PK profile of IgG1 using a 2 compartment model, with k10 (clearance constant) at 0.006 h-1, Vc (plasma vol) 40 mL·kg-1 and 5 kg bodyweight.

FIG. 25: Plasma concentration-time profiles following multiple i.v. doses of IgG1-hDR5-05-E430G+IgG1-hDR5-01-G56T-E430G in female cynomolgus monkeys. Four dose groups were tested with two animals each: 0.1 mg/kg (animals 105 and 106), 0.5 mg/kg (animals 107 and 108), 5 mg/kg (animals 109 and 110) and 25 mg/kg (animals 111 and 112).

FIG. 26: Mean plasma concentration-time profiles following once-weekly intravenous doses of IgG1-hDR5-05-E430G+IgG1-hDR5-01-G56T-E430G in male and female cynomolgus monkeys. Four dose groups were tested with five male and five female animals each: 0, 2, 10, and 50 mg/kg. Graphs represent plasma concentration-time profiles for IgG1-hDR5-01-G56T-E430G (left) and IgG1-hDR5-05-E430G (right) after dosing days 1 (top) and 29 (bottom).

FIG. 27: Plasma concentration-time profiles following first intravenous dose of IgG1-hDR5-05-E430G+IgG1-hDR5-01-G56T-E430G in human cancer patients. Graphs represent plasma concentration-time profiles for IgG1-hDR5-01-G56T-E430G and IgG1-hDR5-05-E430G after the first i.v. dosing of 15 patients in dose escalation cohorts (0.3 and 3.0 mg/kg). Mean plasma concentration time profile after first i.v. dosing of IgG1-hDR5-01-G56T-E430G and IgG1-hDR5-05-E430G in dose escalation cohorts (0.3, 1.0 and 3.0 mg/kg).

### DETAILED DISCLOSURE OF THE INVENTION

As described herein, the present invention relates to DR5-specific antibodies (also referred to as “anti-DR5 ab” or “antibodies that bind DR5” herein) as defined in any aspect or embodiment herein, for use in treating cancers, such as a solid tumor or a hematological malignancy. In particular, a new dosage regimen for a first and a second anti-DR5 antibody is provided. The dosage regimen provides an efficacious therapeutic regimen for treating cancer and has acceptable tolerability and safety profiles.

### Definitions

The term “DR5”, as used herein, refers to death receptor 5, also known as CD262 and TRAILR2, which is a single-pass type I membrane protein with three extracellular cysteine-rich domains (CRDs), a transmembrane domain (TM) and a cytoplasmic domain containing a death domain (DD). In humans, the amino acid sequence encoding the DR5 protein shown in SEQ ID NO 24, is encoded by a nucleic acid sequence (UniProtKB—014763 TR10B_HUMAN).

The term “immunoglobulin” as used herein, refers to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain (HC) typically is comprised of a heavy chain variable region (abbreviated herein as VH) and a heavy chain constant region (CH). The heavy chain constant region of IgG antibodies typically is comprised of three domains, CH1, CH2, and CH3. The heavy chains are inter-connected via disulfide bonds in the so-called “hinge region”. Each light chain (LC) typically is comprised of a light chain variable region (abbreviated herein as VL) and a light chain constant region (CL). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901 917 (1987)). Unless otherwise stated or contradicted by context, reference to amino acid positions in the present invention is according to the EU-numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242).

The term “hinge region” as used herein is intended to refer to the hinge region of an immunoglobulin heavy chain. Thus, for example the hinge region of a human IgG1 antibody corresponds to amino acids 216-230 according to the Eu numbering.

The term “CH2 region” or “CH2 domain” as used herein is intended to refer the CH2 region of an immunoglobulin heavy chain. Thus, for example the CH2 region of a human IgG1 antibody corresponds to amino acids 231-340 according to the Eu numbering. However, the CH2 region may also be any of the other isotypes or allotypes as described herein.

The term “CH3 region” or “CH3 domain” as used herein is intended to refer to the CH3 region of an immunoglobulin heavy chain. Thus, for example the CH3 region of a human IgG1 antibody corresponds to amino acids 341-447 according to the Eu numbering. However, the CH3 region may also be any of the other isotypes or allotypes as described herein.

The term “fragment crystallizable region”, “Fc region”, “Fc fragment” or “Fc domain”, which may be used interchangeably herein, refers to an antibody region comprising, arranged from amino-terminus to carboxy-terminus, at least a hinge region, a CH2 domain and a CH3 domain. An Fc region of an IgG1 antibody can, for example, be generated by digestion of an IgG1 antibody with papain. The Fc region of an antibody may mediate the binding of the immunoglobulin to host tissues or factors, including various cells of the immune system (such as effector cells) and components of the complement system such as C1q, the first component in the classical pathway of complement activation.

The term “Fab fragment” in the context of the present invention, refers to a fragment of an immunoglobulin molecule, which comprises the variable regions of the heavy chain and light chain as well as the constant region of the light chain and the CH1 region of the heavy chain of an immunoglobulin. The “CH1 region” refers e.g. to the region of a human IgG1 antibody corresponding to amino acids 118-215 according to the Eu numbering. Thus, the Fab fragment comprises the binding region of an immunoglobulin.

The term “antibody” (Ab) in the context of the present invention refers to an immunoglobulin molecule, a fragment of an immunoglobulin molecule, or a derivative of either thereof, which has the ability to specifically bind to an antigen. The antibody of the present invention comprises an Fc-domain of an immunoglobulin and an antigen-binding region. An antibody generally contains two CH2-CH3 regions and a connecting region, e.g. a hinge region, e.g. at least an Fc-domain. Thus, the antibody of the present invention may comprise an Fc region and an antigen-binding region. The variable regions of the heavy and light chains of the immunoglobulin molecule contain a binding domain that interacts with an antigen. The constant or “Fc” regions of the antibodies may mediate the binding of the immunoglobulin to host tissues or factors, including various cells of the immune system (such as effector cells) and components of the complement system such as Clq, the first component in the classical pathway of complement activation. An antibody may also be a multispecific antibody, such as a bispecific antibody or similar molecule. The term “bispecific antibody” refers to an antibody having specificities for at least two different, typically non-overlapping, epitopes. Such epitopes may be on the same or different targets. If the epitopes are on different targets, such targets may be on the same cell or different cells or cell types. As indicated above, unless otherwise stated or clearly contradicted by the context, the term antibody herein includes fragments of an antibody which comprise at least a portion of an Fc-region and which retain the ability to specifically bind to the antigen. Such fragments may be provided by any known technique, such as enzymatic cleavage, peptide synthesis and recombinant expression techniques. It has been shown that the antigen-binding function of an antibody may be performed by fragments of a full-length antibody. Examples of binding fragments encompassed within the term “Ab” or “antibody” include, without limitation, monovalent antibodies (described in WO2007059782 by Genmab); heavy-chain antibodies, consisting only of two heavy chains and naturally occurring in e.g. camelids (e.g., Hamers-Casterman (1993) Nature 363:446); ThioMabs (Roche, WO2011069104), strand-exchange engineered domain (SEED or Seed-body) which are asymmetric and bispecific antibody-like molecules (Merck, WO2007110205); Triomab (Pharma/Fresenius Biotech, Lindhofer et al. 1995 J Immunol 155:219; WO2002020039); FcΔAdp (Regeneron, WO2010151792), Azymetric Scaffold (Zymeworks/Merck, WO2012/058768), mAb-Fv (Xencor, WO2011/028952), Xmab (Xencor), Dual variable domain immunoglobulin (Abbott, DVD-Ig, U.S. Pat. No. 7,612,181); Dual domain double head antibodies (Unilever; Sanofi Aventis, WO20100226923), Di-diabody (ImClone/Eli Lilly), Knobs-into-holes antibody formats (Genentech, WO9850431); DuoBody antibodies (Genmab, WO 2011/131746); Bispecific IgG1 and IgG2 (Pfizer/Rinat, WO11143545), DuetMab (MedImmune, US2014/0348839), Electrostatic steering antibody formats (Amgen, EP1870459 and WO 2009089004; Chugai, US201000155133; Oncomed, W02010129304A2), CrossMAbs (Roche, WO2011117329), LUZ-Y (Genentech), BicIonic (Merus, WO2013157953), Dual Targeting domain antibodies (GSK/Domantis), Two-in-one Antibodies or Dual action Fabs recognizing two targets (Genentech, NovImmune, Adimab), Cross-linked Mabs (Karmanos Cancer Center), covalently fused mAbs (AIMM), CovX-body (CovX/Pfizer), FynomAbs (Covagen/Janssen ilag), DutaMab (Dutalys/Roche), iMab (MedImmune), IgG-like Bispecific (ImClone/Eli Lilly, Shen, J., et al. J Immunol Methods, 2007. 318 (1-2): p. 65-74), TIG-body, DIG-body and PIG-body (Pharmabcine), Dual-affinity retargeting molecules (Fc-DART or Ig-DART, by Macrogenics, WO/2008/157379, WO/2010/080538), BEAT (Glenmark), Zybodies (Zyngenia), approaches with common light chain (Crucell/Merus, U.S. Pat. No. 7,262,028) or common heavy chains ( [symbol] [symbol] Bodies by NovImmune, WO2012023053), as well as fusion proteins comprising a polypeptide sequence fused to an antibody fragment containing an Fc-region like scFv-fusions, like BsAb by ZymoGenetics/BMS, HERCULES by Biogen Idec (U5007951918), SCORPIONS by Emergent BioSolutions/Trubion and Zymogenetics/BMS, Ts2Ab (MedImmune/AZ (Dimasi, N., et al. J Mol Biol, 2009. 393 (3): p. 672-92), scFv fusion by Genetech/Roche, scFv fusion by Novartis, scFv fusion by Immunomedics, scFv fusion by Changzhou Adam Biotech Inc (CN 102250246), TvAb by Roche (WO 2012025525, WO 2012025530), mAb² by f-Star (WO2008/003116), and dual scFv-fusions, and like Fc fusions by HERA technology of Apogenix, nanobody-Fc fusions (such as from INHIBRX), MultYmab and MultYbody by JN Biosciences, Stradobody by Gliknik and Zybodies by Zyngenia. It also should be understood that the term antibody, unless specified otherwise, also includes polyclonal antibodies, monoclonal antibodies (such as human monoclonal antibodies), antibody mixtures (recombinant polyclonals) for instance generated by technologies exploited by Symphogen and Merus (Oligoclonics), multimeric Fc proteins as described in WO2015/158867, fusion proteins as described in WO2014/031646 and antibody-like polypeptides, such as chimeric antibodies and humanized antibodies. An antibody as generated can potentially possess any isotype.

The term “human antibody”, as used herein, refers to antibodies having variable and constant regions derived from human germline immunoglobulin sequences. The human antibodies of the invention may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another species, such as a mouse, have been grafted onto human framework sequences.

The term “chimeric antibody”, as used herein, refers to an antibody in which both chain types i.e. heavy chain and light chain are chimeric as a result of antibody engineering. A chimeric chain is a chain that contains a foreign variable domain (originating from a non-human species, or synthetic or engineered from any species including human) linked to a constant region of human origin.

The term “humanized antibody, as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “isotype”, as used herein, refers to the immunoglobulin class (for instance IgG1, IgG2, IgG3, IgG4, IgD, IgA1, IgA2, IgE, or IgM) that is encoded by heavy chain constant region genes. To produce a canonical antibody, each heavy chain isotype is to be combined with either a kappa (κ) or lambda (λ) light chain.

The term “allotype”, as used herein, refers to the amino acid variation within one isotype class in the same species. The predominant allotype of an antibody isotype varies between ethnicity individuals. The known allotype variations within the IgG1 isotype of the heavy chain result from 4 amino acid substitutions in the antibody frame. In one embodiment the antibody of the invention is of the IgG1m(f) allotype as defined in SEQ ID NO 46. In one embodiment of the invention the first and second antibody of the invention is of the IgG1m(f) allotype as defined in SEQ ID NO 46, wherein at least one amino acid substitution has been introduced. In one embodiment of the invention the first and second antibody of the invention is of the IgG1m(f) allotype as defined in SEQ ID NO 46, wherein at most five amino acid substitutions has been introduced, such as four amino acid substitutions, such as three amino acid substitutions, such as two amino acid substitutions.

The terms “monoclonal antibody”, “monoclonal Ab”, “monoclonal antibody composition”, “mAb”, or the like, as used herein refer to a preparation of Ab molecules of single molecular composition. A monoclonal antibody composition displays a single binding specificity and affinity for a particular epitope. Accordingly, the term “human monoclonal antibody” refers to Abs displaying a single binding specificity which have variable and constant regions derived from human germline immunoglobulin sequences. The human mAbs may be generated by a hybridoma which includes a B cell obtained from a transgenic or transchromosomal non-human animal, such as a transgenic mouse, having a genome comprising a human heavy chain transgene repertoire and a human light chain transgene repertoire, rearranged to produce a functional human antibody and fused to an immortalized cell. Alternatively, the human mAbs may be generated recombinantly.

The term “full-length antibody” when used herein, refers to an antibody (e.g., a parent or variant antibody) which contains all heavy and light chain constant and variable domains corresponding to those that are normally found in a wild-type antibody of that class or isotype.

The term “oligomer” as used herein, refers to a molecule that consists of more than one but a limited number of monomer units (e.g. antibodies) in contrast to a polymer that, at least in principle, consists of an unlimited number of monomers. Exemplary oligomers are dimers, trimers, tetramers, pentamers and hexamers. Greek prefixes are often used to designate the number of monomer units in the oligomer, for example a tetramer being composed of four units and a hexamer of six units. Likewise, the term “oligomerization”, as used herein, is intended to refer to a process that converts molecules to a finite degree of polymerization. Herein, it is observed, that antibodies and/or other dimeric proteins comprising target-binding regions according to the invention can form oligomers, such as hexamers, via non-covalent association of Fc-regions after target binding, e.g., at a cell surface.

The term “antigen-binding region”, “antigen binding region”, “binding region” or antigen binding domain, as used herein, refers to a region of an antibody which is capable of binding to the antigen. This binding region is typically defined by the VH and VL domains of the antibody which may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). The antigen can be any molecule, such as a polypeptide, e.g. present on a cell, bacterium, or virion or in solution. The terms “antigen” and “target” may, unless contradicted by the context, be used interchangeably in the context of the present invention.

The term “target”, as used herein, refers to a molecule to which the antigen binding region of the antibody binds. The target includes any antigen towards which the raised antibody is directed. The term “antigen” and “target” may in relation to an antibody be used interchangeably and constitute the same meaning and purpose with respect to any aspect or embodiment of the present invention.

The term “epitope” means a protein determinant capable of specific binding to an antibody. Epitopes usually consist of surface groupings of building blocks such as amino acids, sugar side chains or a combination thereof and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding and other amino acid residues, which are not directly involved in the binding, such as amino acid residues which are effectively blocked by the specifically antigen binding peptide (in other words, the amino acid residue is within the footprint of the specifically antigen binding peptide).

The term “binding” as used herein refers to the binding of an antibody to a predetermined antigen or target, typically with a binding affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less. Binding affinity may be determined by for instance surface plasmon resonance (SPR) technology in a BIAcore 3000 instrument using the antigen as the ligand and the antibody as the analyte or vice versa, and binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its affinity for binding to a non-specific antigen (e.g., BSA, casein) other than the predetermined antigen or a closely-related antigen. The amount with which the affinity is lower is dependent on the K_{D} of the antibody, so that when the K_{D} of the antibody is very low (that is, the antibody is highly specific), then the degree with which the affinity for the antigen is lower than the affinity for a non-specific antigen may be at least 10,000-fold. The term “K_{D}” (M), as used herein, refers to the dissociation equilibrium constant of a particular antibody-antigen interaction, and is obtained by dividing k_{d} by kₐ.

The term “k_{d}” (sec⁻¹), as used herein, refers to the dissociation rate constant of a particular antibody-antigen interaction. Said value is also referred to as the k_{off} value or off-rate.

The term “kₐ” (M⁻¹×sec⁻¹), as used herein, refers to the association rate constant of a particular antibody-antigen interaction. Said value is also referred to as the k₀ₙ value or on-rate.

The term “K_{A}” (M⁻¹), as used herein, refers to the association equilibrium constant of a particular antibody-antigen interaction and is obtained by dividing kₐ by k_{d}.

As used herein, the term “affinity” is the strength of binding of one molecule, e.g. an antibody, to another, e.g. a target or antigen, at a single site, such as the monovalent binding of an individual antigen binding site of an antibody to an antigen.

As used herein, the term “avidity” refers to the combined strength of multiple binding sites between two structures, such as between multiple antigen binding sites of antibodies simultaneously interacting with a target. When more than one binding interactions are present, the two structures will only dissociate when all binding sites dissociate, and thus, the dissociation rate will be slower than for the individual binding sites, and thereby providing a greater effective total binding strength (avidity) compared to the strength of binding of the individual binding sites (affinity).

The term “hexamerization enhancing mutation”, as used herein, refers to a mutation of an amino acid position corresponding to E430, E345 or 5440, with the proviso that the mutation in 5440 is 5440Y or 5440W in human IgG1 according to Eu numbering. The hexamerization enhancing mutation strengthens Fc-Fc interactions between neighbouring IgG1 antibodies that are bound to a cell surface target, resulting in enhanced hexamer formation of the target-bound antibodies, while the antibody molecules remain monomeric in solution as described in WO2013/004842; WO2014/108198.

The term “apoptosis”, as used herein refers to the process of programmed cell death (PCD) that may occur in a cell. Biochemical events lead to characteristic cell changes (morphology) and death. These changes include blebbing, cell shrinkage, phosphatidylserine exposure, loss of mitochondrial function, nuclear fragmentation, chromatin condensation, caspase activation, and chromosomal DNA fragmentation. In a particular embodiment, apoptosis by one or more agonistic anti-DR5 antibodies may be determined using caspase-3/7 activation assays or phosphatidylserine exposure. Anti-DR5 antibody at a fixed concentration of e.g. 1 μg/mL may be added to adhered cells and incubated for 1 to 24 hours. Caspase-3/7 activation can be determined by using special kits for this purpose, such as the PE Active Caspase-3 Apoptosis Kit of BD Pharmingen (Cat no 550914) or the Caspase-Glo 3/7 assay of Promega (Cat no G8091). Phosphatidylserine exposure and cell death can be determined by using special kits for this purpose, such as the FITC Annexin V Apoptosis Detection Kit I from BD Pharmingen (Cat no 556547).

The term “programmed cell-death” or “PCD”, as used herein refers to the death of a cell in any form mediated by an intracellular signaling, e.g. apoptosis, autophagy or necroptosis.

The term “Annexin V”, as used herein, refers to a protein of the annexin group that binds phosphatidylserine (PS) on the cell surface.

The term “caspase activation”, as used herein, refers to cleavage of inactive pro-forms of effector caspases by initiator caspases, leading to their conversion into effector caspases, which in turn cleave protein substrates within the cell to trigger apoptosis.

The term “caspase-dependent programmed cell death”, as used herein refers to any form of programmed cell death mediated by caspases. In a particular embodiment, caspase-dependent programmed cell death by one or more agonistic anti-DR5 antibodies may be determined by comparing the viability of a cell culture in the presence and absence of pan-caspase inhibitor Z-Val-Ala-DL-Asp-fluoromethylketone (Z-VAD-FMK). Pan-caspase inhibitor Z-VAD-FMK (5 μM end concentration) may be added to cells in incubated for one hour at 37° C.. Next, antibody concentration dilution series (e.g. starting from e.g. 20,000 ng/mL to 0.05 ng/mL final concentrations in 5-fold dilutions) may be added and incubated for 3 days at 37° C. Cell viability can be quantified using special kits for this purpose, such as the CellTiter-Glo luminescent cell viability assay of Promega (Cat no G7571).

The term “cell viability”, as used herein refers to the presence of metabolically active cells. In a particular embodiment, cell viability after incubation with one or more agonistic anti-DR5 antibodies can be determined by quantifying the ATP present in the cells. Antibody concentration dilution series (e.g. starting from e.g. 20,000 ng/mL to 0.05 ng/mL final concentration in 5-fold dilutions) may be added to cells, medium may be used as negative control and 5 μM staurosporine may be used as positive control for the induction of cell death. After 3 days incubation cell viability may be quantified using special kits for this purpose, such as the CellTiter-Glo luminescent cell viability assay of Promega (Cat no G7571) or ATPlite 1step Luminescence Assay System of Perkin Elmer (Cat no 6016739). The percentage viable cells can be calculated using the following formula: % viable cells=[(luminescence antibody sample−luminescence staurosporine sample)/(luminescence no antibody sample−luminescence staurosporine sample)]*100.

The term “antibody binding DR5”, “anti-DR5 antibody”, “DR5-binding antibody”, “DR5-specific antibody”, “DR5 antibody” “antibody that binds DR5” or “antibodies that bind DR5” which may be used interchangeably herein, refers to any antibody binding an epitope on the extracellular part of DR5.”

The term “agonist” as used herein, refers to a molecule such as an anti-DR5 antibody that is able to trigger a response in a cell when bound to DR5, wherein the response may be programmed cell death. That the anti-DR5 antibody is agonistic is to be understood as that the antibody stimulates, activates or clusters DR5 as the result from the anti-DR5 antibody binding to DR5. An agonistic anti-DR5 antibody comprising an amino acid mutation in the Fc region according to the present invention bound to DR5 results in DR5 stimulation, clustering or activation of the same intracellular signaling pathways as TRAIL bound to DR5.

In a particular embodiment, the agonistic activity of one or more antibodies can be determined by incubating target cells for 3 days with an antibody concentration dilution series (e.g. from 20,000 ng/mL to 0.05 ng/mL final concentrations in 5-fold dilutions). The antibodies may be added directly when cells are seeded, or alternatively the cells are first incubated for 4h at 37° C. before adding the antibody samples. The agonistic activity i.e. the agonistic effect can be quantified by measuring the amount of viable cells using special kits for this purpose, such as the CellTiter-Glo luminescent cell viability assay of Promega (Cat no G7571) or or ATPlite 1step Luminescence Assay System of Perkin Elmer (Cat no 6016739).

The terms “DR5-positive” and “DR5-expressing” as used herein, refers to tissues or cells which show binding of a DR5-specific antibody which can be measured with e.g. flow cytometry or immunohistochemistry.

A “variant” or “antibody variant” of the present invention is an antibody molecule which comprises one or more mutations as compared to a “parent” antibody. Exemplary parent antibody formats include, without limitation, a wild-type antibody, a full-length antibody or Fc-containing antibody fragment, a bispecific antibody, a human antibody, humanized antibody, chimeric antibody or any combination thereof.

The term “amino acid substitution” embraces a substitution into any one or the other nineteen natural amino acids, or into other amino acids, such as non-natural amino acids. For example, an amino acid may be substituted for another conservative or non-conservative amino acid. Amino acid residues may also be divided into classes defined by alternative physical and functional properties.

### Amino Acid Residue Classes for Conservative Substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and Gln (Q) |
| Aliphatic Uncharged Residues | Gly (G), Ala (A), Val (V), Leu (L), and Ile (I) |
| Non-polar Uncharged Residues | Cys (C), Met (M), and Pro (P) |
| Aromatic Residues | Phe (F), Tyr (Y), and Trp (W) |

### Alternative Conservative Amino Acid Residue Substitution Classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, |
| | V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, |
| formation | P, and T |
| Flexible residues | Q, T, K, S, G, D, E, and R |

In the context of the present invention, a substitution in a variant is indicated as:

Original amino acid—position-substituted amino acid;

The three letter code, or one letter code, are used, including the codes Xaa and X to indicate amino acid residue. Accordingly, the notation “E345R” or “Glu345Arg” means, that the variant comprises a substitution of Glutamic acid with Arginine in the variant amino acid position corresponding to the amino acid in position 345 in the parent antibody.

Where a position as such is not present in an antibody, but the variant comprises an insertion of an amino acid, for example: Position—inserted amino acid; the notation, e.g., “448E” is used. Such notation is particular relevant in connection with modification(s) in a series of homologous polypeptides or antibodies.

For a modification where the original amino acid(s) and/or substituted amino acid(s) may comprise more than one, but not all amino acid(s), e.g., the substitution of Glutamic acid for Arginine, Lysine or Tryptophan in position 345: “Glu345Arg,Lys,Trp” or “E345R,K,W” or “E345R/K/W” or “E345 to R, K or W” may be used interchangeably in the context of the invention. Furthermore, the term “a substitution” embraces a substitution into any one of the other nineteen natural amino acids, or into other amino acids, such as non-natural amino acids. For example, a substitution of amino acid E in position 345 includes each of the following substitutions: 345A, 345C, 345D, 345G, 345H, 345F, 345I, 345K, 345L, 345M, 345N, 345Q, 345R, 345S, 345T, 345V, 345W, and 345Y. This is, by the way, equivalent to the designation 345X, wherein the X designates any amino acid. These substitutions can also be designated E345A, E345C, etc, or E345A,C, ect, or E345A/C/ect. The same applies to analogy to each and every position mentioned herein, to specifically include herein any one of such substitutions.

For the purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment).

For the purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et a/., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

The sequence of CDR variants may differ from the sequence of the CDR of the parent antibody sequences through mostly conservative, physical or functional amino acids substitutions at most 5 mutations or substitutions selected from conservative, physical or functional amino acids in total across the six CDR sequences of the antibody binding region, such as at most 4 mutations or substitutions selected from conservative, physical or functional amino acids, such as at most 3 mutations or substitutions selected from conservative, physical or functional amino acids, such as at most 2 mutations selected from conservative, physical or functional amino acids or substitutions, such as at most 1 mutation or substitution selected from a conservative, physical or functional amino acid, in total across the six CDR sequences of the antibody binding region. The conservative, physical or functional amino acids are selected from the 20 natural amino acids found i.e, Arg (R), His (H), Lys (K), Asp (D), Glu (E), Ser (S), Thr (T), Asn (N), Gln (Q), Cys (C), Gly (G), Pro (P), Ala (A), Ile (I), Leu (L), Met (M), Phe (F), Trp (W), Tyr (Y) and Val (V).

The sequence of CDR variants may differ from the sequence of the CDR of the parent antibody sequences through mostly conservative, physical or functional amino acids substitutions; for instance at least about 75%, about 80% or more, about 85% or more, about 90% or more, about 95% or more (e.g., about 75-99%, such as about 92%, 93% or 94%) of the substitutions in the variant are mutations or substitutions selected from conservative, physical or functional amino acids residue replacements. The conservative, physical or functional amino acids are selected from the 20 natural amino acids found i.e, Arg (R), His (H), Lys (K), Asp (D), Glu (E), Ser (S), Thr (T), Asn (N), Gln (Q), Cys (C), Gly (G), Pro (P), Ala (A), Ile (I), Leu (L), Met (M), Phe (F), Trp (W), Tyr (Y) and Val (V).

An amino acid or segment in one sequence that “corresponds to” an amino acid or segment in another sequence is one that aligns with the other amino acid or segment using a standard sequence alignment program such as ALIGN, ClustalW or similar, typically at default settings. Hence a standard sequence alignment program can be used to identify which amino acid in an e.g. immunoglobulin sequence corresponds to a specific amino acid in e.g. human IgG1. Further a standard sequence alignment program can be used to identify sequence identity e.g. a sequence identity to SEQ ID NO:46 of at least 80%, or 85%, 90%, or at least 95%.

The term “vector,” as used herein, refers to a nucleic acid molecule capable of inducing transcription of a nucleic acid segment ligated into the vector. One type of vector is a “plasmid”, which is in the form of a circular double stranded DNA loop. Another type of vector is a viral vector, wherein the nucleic acid segment may be ligated into the viral genome. Certain vectors are capable of autonomous replication in a host cell into which they are introduced (for instance bacterial vectors having a bacterial origin of replication and episomal mammalian vectors). Other vectors (such as non-episomal mammalian vectors) may be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present invention is intended to include such other forms of expression vectors, such as viral vectors (such as replication defective retroviruses, adenoviruses and adeno-associated viruses), which serve equivalent functions.

The term “recombinant host cell” (or simply “host cell”), as used herein, is intended to refer to a cell into which an expression vector has been introduced. It should be understood that such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, transfectomas, such as CHO-S cells, HEK-293F cells, Expi293F cells, PER.C6, NS0 cells, and lymphocytic cells, and prokaryotic cells such as E. coli and other eukaryotic hosts such as plant cells and fungi, as well as prokaryotic cells such as E. coli.

As used herein, a “derivative” of a drug is a compound that is derived or derivable, by a direct chemical reaction, from the drug. As used herein, an “analog” or “structural analog” of a drug is a compound having a similar structure and/or mechanism of action to the drug but differing in at least one structural element. “Therapeutically active” analogs or derivatives of a parent drug such may have a similar or improved therapeutic efficacy as compared to the parent drug but may differ in, e.g., one or more of stability, solubility, toxicity, and the like.

“Treatment” refers to the administration of an effective amount of a therapeutically active compound as described herein to a subject with the purpose of easing, ameliorating, arresting or eradicating (curing) symptoms or disease states of the subject.

As used herein, “maintenance therapy” means therapy for the purpose of avoiding or delaying the cancer's progression or return. Typically, if a cancer is in complete remission after the initial treatment, maintenance therapy can be used to avoid or delay return of the cancer. If the cancer is advanced and complete remission has not been achieved after the initial treatment, maintenance therapy can be used to slow the growth of the cancer, e.g., to lengthen the life of the patient.

As used herein, the term “subject” is typically a human, to whom a first and second antibody binding to DR5 is administered, including for instance human patients diagnosed as having a cancer that may be treated by killing of DR5-expressing cancer cells, directly or indirectly.

An “effective amount” or “therapeutically effective amount” refers to an amount effective, at dosages and for periods of time necessary, to achieve a desired therapeutic result. A therapeutically effective amount of a first and second anti-DR5 antibody may vary according to factors such as the disease state, age, sex, and weight of the individual, and the ability of the first and second anti-DR5 antibody to elicit a desired response in the individual. A therapeutically effective amount is also one in which any toxic or detrimental effects of the first and second anti-DR5 are outweighed by the therapeutically beneficial effects.

The term a “cycle” or “cycle of treatment” describes a period of treatment followed by a period of rest (no treatment) that is repeated on a regular schedule. For example, treatment given on day 1 followed by 13 days of rest is one treatment cycle of 14 days. When this cycle is repeated multiple times on a regular schedule, it makes up a course of treatment. In one embodiment the treatment is administered on day 1 of a 14 days cycle. In one embodiment the treatment is administered on day 1 and day 8 of a 14 days cycle.

The term “Ctrough” describes the drug serum concentration at the end of the dosing interval. Thus, Ctrough is the lowest concentration reached by a drug before the next dose is administered.

The term “Therapeutic Index” (TI) describes the ratio of the dose of drug that causes adverse effects at an incidence/severity not compatible with the targeted indication (e.g. toxic dose in 50% of subjects, TD50) to the dose that leads to the desired pharmacological effect (e.g. efficacious dose in 50% of subjects, ED50).

As used herein, a “resistant”, “treatment-resistant” or “refractory” cancer, tumor or the like, means a cancer or tumor in a subject, wherein the cancer or tumor did not respond to treatment with a therapeutic agent from the onset of the treatment (herein referred to as “native resistance”) or initially responded to treatment with the therapeutic agent but became non-responsive or less responsive to the therapeutic agent after a certain period of treatment (herein referred to as “acquired resistance”), resulting in progressive disease. For solid tumors, also an initial stabilization of disease represents an initial response. Other indicators of resistance include recurrence of a cancer, increase of tumor burden, newly identified metastases or the like, despite treatment with the therapeutic agent. Whether a tumor or cancer is, or has a high tendency of becoming resistant to a therapeutic agent, can be determined by a person of skill in the art. For example, the National Comprehensive Cancer Network (NCCN, www.nccn.org) and European Society for Medical Oncology (ESMO, www.esmo.org/Guidelines) provide guidelines for assessing whether a specific cancer responds to treatment.

### Specific Embodiments of the Invention

As explained above, the invention is directed to a combination treatment involving a first antibody that binds to DR5 and a second antibody that binds to DR5, wherein the dosage regimen has been improved to reach an efficacious drug exposure within a shorter period of time to provide for a more effective treatment compared to a biweekly dosage regimen.

In one aspect, the present invention relates to a method of treating a solid tumor or a hematological malignancy in a subject, the method comprising administering to a subject in need thereof a first antibody that binds DR5 and a second antibody that binds DR5, or a pharmaceutically acceptable salt thereof, wherein the first antibody and the second antibody is administered on, i) day 1 and day 8 of a 14-days cycle for the first four cycles (intensified); or ii) day 1 and day 8 of a first 14-days cycle (priming); or iii) day 1 of a 14-days cycle (priming); or iv) day 1 of a first and second 14-days cycle (priming); followed by administration on day 1 of a 14-days cycle. Thus, the present invention provides for a method of treating a solid tumor or a hematological malignancy in a subject wherein the first and second antibody that binds DR5 is administered to the subject based on an intensified regimen which is on day 1 and day 8 of a 14-days cycle for the first four cycles), this may allow for a higher pre-dose Ctrough value and an improved therapeutic index, thereby allowing Ctrough to be consistently maintained during the course of the treatment duration; in subsequent doses, the subject may then continue treatment with the first and second antibody that binds DR5 based on a dosage regimen where the first and second antibody is administered on day 1 of a 14-days cycle (Q2W). The present invention also provides for a method of treating a solid tumor or a hematological malignancy in a subject wherein the first and second antibody that binds DR5 is administered on day 1 and day 8 of a first 14-days cycle and where the dose administered is a priming dose, which may allow for desensitization of the subjects to the therapy and reduce potential toxicities of higher doses of treatment, the subject may then continue treatment with the first and second antibody that binds DR5 based on a dosage regimen were the first and second antibody is administered on day 1 of a 14-days cycle (Q2W). The present invention also provides for a method of treating a solid tumor or a hematological malignancy in a subject wherein the first and second antibody that binds DR5 is administered on day 1 of a 14-day cycle, where the dose administered is a priming dose, which may allow for incremental build-up of the exposure to the drug, and may reduce the incidence and severity of perceived toxicities which may occur with the administration of higher doses. Thus, administration of a priming dose which is lower dose that the dose administered in the following treatment cycles and may improve drug tolerability. The subject may continue treatment based on a bi-weekly dosage regimen where the drug product i.e. the first and second antibody binding to DR5 is administered on day 1 of a 14-days cycle (Q2W).

Preferred anti-DR5 antibodies are characterized by DR5 binding properties, variable or hypervariable sequences, or a combination of binding and sequence properties, set out in the aspects and embodiments below. Most preferred are the specific anti-DR5 antibodies comprising VH region and VL region CDRs, VH and/or VL sequences described in Table 2 of particular interest are antibodies sharing one or more DR5 binding properties or CDRs, VH and/or VL sequences with an antibody selected from the group consisting of antibody DR5-01 and antibody DR5-05 and or a variant of any thereof.

In one embodiment, the anti-DR5 antibody comprises a variable heavy chain (VH) region and a variable light chain (VL) region, wherein the VH region and VL region comprises the CDR sequences selected from the group consisting of
(a) a VH region comprising the CDR1, CDR2, and CDR3 sequences of SEQ ID Nos.: 1, 8, and 3, respectively; and a VL region comprising the CDR1, CDR2, and CDR3 sequences of SEQ ID Nos.: 5, FAS, and 6, respectively, [DR5-01-G56T];
(b) a VH region comprising the CDR1, CDR2, and CDR3 sequences of SEQ ID Nos.: 1, 2, and 3, respectively; and a VL region comprising the CDR1, CDR2, and CDR3 sequences of SEQ ID Nos.: 5, FAS, and 6, respectively, [DR5-01];
(c) a VH region comprising the CDR1, CDR2, and CDR3 sequences of SEQ ID Nos.: 10, 2, and 11, respectively, and a VL region comprising the CDR1, CDR2, and CDR3 sequences of SEQ ID Nos.: 13, RTS, and 14, respectively [DR5-05];
(d) a VH region comprising the CDR1, CDR2, and CDR3 sequences of SEQ ID Nos.:16, 17, and 18, respectively; and a VL region comprising the CDR1, CDR2, and CDR3 sequences of SEQ ID Nos.: 21, GAS, and 22, respectively [cona];
(e) a variant of any of said antibodies defined in (a) to (d), wherein said variant preferably has at most 1, 2 or 3 amino acid modifications, more preferably amino acid substitutions, such as conservative amino acid substitutions, across the six CDR sequences. Thus, in one embodiment the first and the second antibody may be selected from an antibody as described in (a) to (d) where the first and second antibody is not the same.

In one embodiment the first or second antibody that binds DR5 comprises a variable heavy chain (VH) region and a variable light chain (VL) region wherein the VH region comprises the CDR1, CDR2 and CDR3 sequences of SEQ ID Nos: 1, 8, and 3 respectively; and wherein the VL region comprises the CDR1, CDR2 and CDR3 sequences of SEQ ID Nos: 5, FAS, and 6 respectively.

In one embodiment the first antibody that binds DR5 comprises a variable heavy chain (VH) region and a variable light chain (VL) region wherein the VH region comprises the CDR1, CDR2 and CDR3 sequences of SEQ ID Nos: 1, 2, and 3 respectively; and wherein the VL region comprises the CDR1, CDR2 and CDR3 sequences of SEQ ID Nos: 5, FAS, and 6 respectively.

In one embodiment the first or second antibody that binds DR5 comprises a variable heavy chain region and a variable light chain region wherein the variable heavy chain region comprises the CDR1, CDR2 and CDR3 sequences of SEQ ID Nos: 10, 2, and 11 respectively; and wherein the variable light chain region comprises the CDR1, CDR2 and CDR3 sequences of SEQ ID Nos: 13, RTS, and 14 respectively.

In one embodiment the second antibody that binds DR5 comprises a variable heavy chain region and a variable light chain region wherein the variable heavy chain region comprises the CDR1, CDR2 and CDR3 sequences of SEQ ID Nos: 16, 17, and 18 respectively; and wherein the variable light chain region comprises the CDR1, CDR2 and CDR3 sequences of SEQ ID Nos: 21, GAS, and 22 respectively.

In one embodiment of the invention, the first or second antibody that binds DR5 comprises a VH region and a VL region selected from the group consisting of:
(a) a VH region comprising SEQ ID No: 9 and a VL region comprising SEQ ID No:7 [DR5-01-G56T];
(b) a VH region comprising SEQ ID No: 4 and a VL region comprising SEQ ID No: 7 [DR5-01]; and
(c) a VH region comprising SEQ ID No: 12 and a VL region comprising SEQ ID No: 15 [DR5-05].

In one preferred embodiment of the invention, the first antibody that binds DR5 is the antibody having the VH region CDR1, CDR2 and CDR3 amino acid sequences set forth in SEQ ID Nos 1, 8, and 3, respectively; and the VL region CDR1, CDR2 and CDR3 amino acid sequence set forth in SEQ ID Nos 5, FAS, and 6, respectively, [DR5-01-G56T] and the second antibody that binds DR5 is the antibody having the VH region CDR1, CDR2 and CDR3 amino acid sequences set forth in SEQ ID Nos 10, 2, and 11, respectively; and the VL region CDR1, CDR2 and CDR3 amino acid sequence set forth in SEQ ID Nos 13, RTS, and 14, respectively, [DR5-05]. For example, the first antibody that binds DR5 may comprise a VH region comprising SEQ ID No: 9 and a VL region comprising SEQ ID No: 7 [DR5-01-G56T]; and the second antibody that binds DR5 may comprise a VH region comprising SEQ ID No: 12 and a VL region comprising SEQ ID No: 15 [DR5-05].

In one embodiment of the invention the first and second antibody bind different epitopes on DR5. Hereby are embodiments provided where the antibodies bind different epitopes or require different amino acids within the DR5 sequence (SEQ ID NO 24) for binding to DR5. In one embodiment of the invention the first and second antibody bind non-overlapping epitopes on DR5. That is in one embodiment of the invention the first and second antibodies binding to DR5 do not compete for binding to DR5, thus the first and second antibody may bind DR5 simultaneously.

In a preferred embodiment of the invention, the antibody is a full-length antibody. The antibody may, for example, be a fully human monoclonal IgG1 antibody, such as an IgG1,κ. In one embodiment, the antibody is a full-length antibody.

In one embodiment of the invention the antibody binding to DR5 comprises an Fc region of a human IgG1, wherein the Fc region comprises a mutation which enhances Fc-Fc interactions between antibodies. Mutations which have been shown to enhance Fc-Fc interactions are mutations at an amino acid position corresponding to E430, E345, or 5440 in human IgG1 according to Eu numbering, with the proviso that the mutation in 5440 is 5440Y or S440W. Mutations that enhance Fc-Fc interactions has also been found to enhance hexamerization of antibodies comprising such Fc-Fc enhancing mutations, once such antibodies bind to their target on a cell surface.

In one embodiment of the invention the antibody binding to DR5 comprises an Fc region of human IgG1, wherein the Fc region comprises a mutation at the amino acid position corresponding to E430. In one embodiment the antibody binding to DR5 comprises an Fc region of human IgG1, wherein the Fc region comprises a mutation at the amino acid position corresponding to E345. In one embodiment the antibody binding to DR5 comprises an Fc region of human IgG1, wherein the Fc region comprises a 5440Y or S440W mutation.

In one embodiment of the invention the first and/or second antibody comprises a mutation at the amino acid position corresponding to E430 in human IgG1 according to Eu numbering, wherein the mutation is selected from the group consisting of: E430G, E430S, E430F and E430T.

In one embodiment of the invention the first and/or second antibody comprises a mutation at the amino acid position corresponding to E345 in human IgG1 according to EU numbering, wherein the mutation is selected form the group consisting of: E345K, E345Q, E345R and E345Y.

In one embodiment of the invention the first and/or second antibody comprises a mutation corresponding to S440Y or S440W in human IgG1 according to Eu numbering.

In one embodiment of the invention the first and second antibody comprises an Fc region of a human IgG1, wherein the Fc region comprises an E430G mutation in human IgG1, wherein the amino acid position is according to the Eu numbering.

In one embodiment of the invention the first or second antibody comprises the heavy chain set forth in SEQ ID NO 30. In one embodiment of the invention the first or second antibody comprises the heavy chain set forth in SEQ ID NO 32.

In one embodiment of the invention the first or second antibody comprises the heavy chain set forth in SEQ ID NO 47. In one embodiment of the invention the first or second antibody comprises the heavy chain set forth in SEQ ID NO 48.

In one embodiment of the invention the first or second antibody comprises the light chain set forth in SEQ ID NO 27. In one embodiment of the invention the first or second antibody comprises the light chain set forth in SEQ ID NO 35.

In one embodiment of the invention the first or second antibody comprises the heavy chain and light chain as set forth in SEQ ID Nos 30 and 27, respectively.

In one embodiment of the invention the first or second antibody comprises the heavy chain and light chain as set forth in SEQ ID Nos 47 and 27, respectively.

In one embodiment of the invention the first or second antibody comprises the heavy chain and light chain as set forth in SEQ ID NOs 32 and 35, respectively.

In one embodiment of the invention the first or second antibody comprises the heavy chain and light chain as set forth in SEQ ID NOs 48 and 35, respectively.

In one embodiment of the invention the first antibody comprises the heavy chain and light chain as set forth in SEQ ID NOs 30 and 27, respectively.

In one embodiment of the invention the first antibody comprises the heavy chain and light chain as set forth in SEQ ID NOs 47 and 27, respectively.

In one embodiment of the invention the second antibody comprises the heavy chain and light chain as set forth in SEQ ID NOs 32 and 35, respectively.

In one embodiment of the invention the second antibody comprises the heavy chain and light chain as set forth in SEQ ID NOs 48 and 35, respectively.

### Therapeutic Applications

The present invention provides for methods of treating a solid tumor or a hematological malignancy in a subject by administering a first and a second antibody binding to DR5 as described herein.

The present invention includes embodiments wherein a subject will be administered a first and a second antibody that binds DR5 where the first and second antibody or a pharmaceutically acceptable salt thereof is administered on i) day 1 and day 8 of a 14-days cycle for the first four cycles; or ii) day 1 and 8 of a first 14-days cycle (priming); or iii) day 1 of a first 14-days cycle (priming) or iii) day 1 of a first and second 14-days cycle (priming); followed by administration on day 1 of a 14-days cycle (Q2W). Followed by the initial dosage schedule according to i), which allows for establishment of more stable Ctrough values and an improved therapeutic index, the subject may receive treatment administered based on a biweekly dosage schedule (Q2W), following the initial dosage schedule according to ii), iii) or iv) which may allow for desensitization of the subjects to the therapy and reduce potential toxicities of higher doses of treatment the subject may receive treatment administered based on a biweekly dosage. The effect of administering a priming dose may mitigate potential transaminase elevations caused by administration of the first and second antibody binding to DR5. Thus, administering a priming dose may reduce, prevent or lessen the induction of transaminase levels by the first and second antibody, such as reduce, prevent or lessen the induction of alanine transaminase (ALT) or aspartate transaminase (AST).

In one embodiment the priming doses administered according to ii)-iv) are in the range of 0.05 mg/kg to 0.15 mg/kg for each of the first and second antibody. In one embodiment the combined priming dose of the first and second antibody is in the range of 0.1 mg/kg to 0.3 mg/kg. In a preferred embodiment the combined priming dose of the first and second antibody is 0.1 mg/kg.

The treatment dose administered following the priming dose is in the range of 0.15 mg/kg to 9 mg/kg for each first and second antibody. In one embodiment following the first or first and second priming dose the subject is administered a treatment dose on a bi-weekly schedule wherein the treatment dose is in the range of 0.15 mg/kg to 9 mg/kg for each first and second antibody. In one embodiment following the first or first and second priming dose the subject is administered a treatment dose on a bi-weekly schedule wherein the treatment dose is in the range of 0.3 mg/kg to 18 mg/kg for the combined dose of th first and second antibody. In a preferred embodiment the treatment dose of the first and second antibody combined is in the range of 0.3 mg/kg to 6 mg/kg. In a more preferred embodiment, the treatment dose of the first and second antibody combined is in the range of 0.3 mg/kg to 3 mg/kg.

The subject to be treated according to a dosage regimen of the present invention is typically a subject expected to benefit from the administration of a first and a second antibody that binds DR5. In separate and specific exemplary embodiments, the subject to be treated according to a dosage regimen of the present invention is selected from:
a subject that has been diagnosed with a solid tumor or cancer,
a subject that has been diagnosed with a hematological malignancy,
a subject suspected of having a tumor or cancer that expresses DR5, or
a subject diagnosed with a cancer which is resistant, or which has a high tendency to become resistant, to certain therapeutic agent(s).

For example, a cancer that expresses DR5 may be a solid tumor expressing DR5 or it may be a DR5-expressing hematological cancer.

In some embodiments, the cancer comprises a solid tumor expressing DR5, and is selected from the group consisting of lung cancer, such as non-small cell lung cancer (NSCLC) and lung squamous cell carcinoma; a gynaecological cancer, such as ovarian cancer, endometrial cancer or cervical cancer; thyroid cancer; a skin cancer, such as melanoma, e.g., malignant melanoma; colorectal cancer, such as colorectal carcinoma and colorectal adenocarcinoma; bladder cancer; bone cancer, such as chondrosarcoma; breast cancer, such as triple-negative breast cancer (TNBC); cancers of the central nervous system, such as glioblastoma, astrocytoma and neuroblastoma; connective tissue cancer; fibroblast cancer; gastric cancer, such as gastric carcinoma; head and neck cancer; kidney cancer; liver cancer, such as hepatocellular carcinoma; muscle cancer; neural tissue cancer; pancreatic cancer, such as pancreatic ductal carcinoma and pancreatic adenocarcinoma; and sarcoma, such as soft tissue sarcoma. In one embodiment, the cancer is melanoma. In one embodiment, the cancer is lung cancer, such as non-small cell lung cancer (NSCLC). In one embodiment, the cancer is sarcoma, such as a sarcoma selected from the group consisting of undifferentiated pleomorphic sarcoma, liposarcoma, leiomyosarcoma, synovial sarcoma, Ewing's sarcoma, osteosarcoma and chondrosarcoma. In one embodiment, the cancer is ovarian cancer. In one embodiment, the cancer is endometrial cancer. In one embodiment, the solid cancer is cervical cancer. In one embodiment, the cancer is thyroid cancer. In one embodiment, the solid cancer is colorectal cancer.

In vitro analysis of the cytotoxic ability of a first and second antibody according to the present invention has shown that antibodies according to the invention show cytotoxic activity in a broad range of solid tumor cell lines, Example 7, Example 8. In a preferred embodiment of the invention the solid tumor is selected from the group consisting of: colorectal cancer (CRC), non-small lung cancer (NSCLC), triple negative breast cancer (TNBC), renal cell carcinoma (RCC), gastric cancer, pancreatic cancer, urothelial cancer, melanoma, brain tumors, ovarian cancer, liver cancer, endometrial cancer, head and neck cancer, and lung mesolthelioma. In one embodiment the solid tumor is a colorectal cancer (CRC). In one embodiment the solid tumor is a non-small lung cancer (NSCLC). In one embodiment the solid tumor is a triple negative breast cancer (TNBC). In one embodiment the solid tumor is a renal cell carcinoma (RCC).

In one embodiment the solid tumor is a gastric cancer. In one embodiment the solid tumor is a pancreatic cancer. In one embodiment the solid tumor is an urothelial cancer.

In some embodiments, the DR5-expressing tumor is a hematological malignancy. In one embodiment the hematological malignancy is selected from the group consisting of: leukemia, including chronic lymphocytic leukemia (CLL) and myeloid leukemia, including acute myeloid leukemia (AML) and chronic myeloid leukemia, lymphoma, Non-Hodgkin lymphoma (NHL), including diffuse large B-cell lymphoma (DLBCL), mantle cell lymphoma (MCL), and follicular lymphoma (FL), or multiple myeloma (MM), Hodgkin Lymphoma or myelodysplastic syndromes.

In one embodiment the hematological malignancy is leukemia. In one embodiment the hematological malignancy is chronic lymphocytic leukemia (CLL). In one embodiment the hematological malignancy is myeloid leukemia. In one embodiment the hematological malignancy is acute myeloid leukemia (AML). In one embodiment the hematological malignancy is chronic myeloid leukemia. In one embodiment the hematological malignancy is lymphoma. In one embodiment the hematological malignancy is Non-Hodgkin lymphoma (NHL). In one embodiment the hematological malignancy is multiple myeloma (MM). In one embodiment the hematological malignancy is Hodgkin Lymphoma. In one embodiment the hematological malignancy is myelodysplastic syndromes.

In vitro analysis of the cytotoxic ability of a first and second antibody according to the present invention has shown that antibodies according to the invention show cytotoxic activity in a broad range of haematological cell lines, Example 9. In a preferred embodiment of the invention the hematological malignancy is selected from the group consisting of AML, DLBCL, FL, MM, and MCL.

In one embodiment of the invention the first and second antibody, or a pharmaceutically acceptable salt thereof, are administered simultaneously, separately, or sequentially. In one embodiment of the invention the first and second antibody, or a pharmaceutically acceptable salt thereof, are administered simultaneously. That is, the first and second antibody may be stored separately, but mixed together to a single solution before administration, so that the first and second antibody may be administered simultaneously. In one embodiment of the invention the first and second antibody, or pharmaceutically acceptable salt thereof, are administered separately. In one embodiment of the invention the first and second antibody, or a pharmaceutically acceptable salt thereof, are administered sequentially. That is, the first antibody may be administered to the subject first followed by administration of the second antibody. Alternatively, the second antibody may be administered to the subject first followed by administration of the first antibody.

In one embodiment of the invention the first or second antibody, or a pharmaceutically acceptable salt thereof, is administered by intravenous infusion.

In one embodiment of the invention the first and second antibody, or a pharmaceutically acceptable salt thereof, are administered by intravenous infusion.

### Protocol

In one aspect, the present invention provides for methods of treating a subject with a solid tumor or a hematological malignancy as described herein wherein the first and second antibody or pharmaceutically acceptable salt thereof are administered at a particular frequency.

The present invention includes embodiments wherein a subject will be administered a first and a second antibody that binds DR5, where the first and second antibody or a pharmaceutically acceptable salt thereof are administered on i) 1 and day 8 of a 14-days cycle for the first four cycles; or ii) day 1 and 8 of a first 14-days cycle (priming); or iii) day 1 of a first 14-days cycle (priming); or iv) day 1 of a first and second 14-days cycle (priming); followed by administration on day 1 of a 14-days cycle (Q2W). Thus following the initial dosage schedule according to i) which allows for establishment of more stable Ctrough values and an improved therapeutic index, the subject may receive treatment administered based on a biweekly dosage schedule, following the initial dosage schedule according to ii) or iii) which may allow for desensitization of the subjects to the therapy and reduce potential toxicities of higher doses of treatment the subject may receive treatment administered based on a biweekly dosage.

In a preferred embodiment the first and second antibody is administered as a single priming dose on day 1 of a 14-day cycle, followed by administration of a treatment dose on day 1 of a 14-day cycle. In a preferred embodiment of the invention the priming dose is 0.05 mg/kg of each of the first and second antibody. In a preferred embodiment of the invention the priming dose is 0.1 mg/kg of the first and second antibody combined.

In on embodiment of the invention the treatment dose administered following the priming dose is within the range of 0.15 mg/kg to 9 mg/kg for each of the first and second antibody. In a preferred embodiment of the invention the treatment dose administered following the priming dose is within the range of 0.15 mg/kg to 3 mg/kg for each of the first and second antibody.

In on embodiment of the invention the treatment dose administered following the priming dose is within the range of 0.3 mg/kg to 18 mg/kg for the combined dose of the first and second antibody. In a preferred embodiment of the invention the treatment dose administered following the priming dose is within the range of 0.3 mg/kg to 6 mg/kg for the combined dose of the first and second antibody. In one embodiment of the invention the treatment dose of the first and second antibody is 0.3 mg/kg. In one embodiment of the invention the treatment dose of the first and second antibody is 0.6 mg/kg. In one embodiment of the invention the treatment dose of the first and second antibody is 1 mg/kg. In one embodiment of the invention the treatment dose of the first and second antibody is 2 mg/kg. In one embodiment of the invention the treatment dose of the first and second antibody is 3 mg/kg. In one embodiment of the invention the treatment dose of the first and second antibody is 4 mg/kg. In one embodiment of the invention the treatment dose of the first and second antibody is 4.5 mg/kg. In one embodiment of the invention the treatment dose of the first and second antibody is 6 mg/kg. In one embodiment of the invention the treatment dose of the first and second antibody is 9 mg/kg. In one embodiment of the invention the treatment dose of the first and second antibody is 12 mg/kg. In one embodiment of the invention the treatment dose of the first and second antibody is 15 mg/kg. In one embodiment of the invention the treatment dose of the first and second antibody is 18 mg/kg. Hereby embodiments are provided wherein the treatment dose is a presented as the combined dose of the first and second antibody.

In one embodiment of the present invention, the first and second antibody binding to DR5, or pharmaceutical acceptable salt thereof, are administered twice for the first four cycles followed by administration on day 1 of a 14-days cycle (Q2W). In one embodiment the first and second antibody or pharmaceutical acceptable salt thereof, are administered twice in the first four cycles, thus the first dose of the first and second antibody may be administered on day 1, 2, 3, 4, 5, 6 or 7 of a 14-days cycle and the second dose of the first and second antibody may be administered on day 8, 9, 10, 11, 12, 12 or 14 of a 14 days cycle followed by administration of the first and second antibody, or a pharmaceutical acceptable salt thereof, on day 1 of a 14-days cycle (Q2W). In one embodiment the first dose of the first and second antibody, or pharmaceutical acceptable salt thereof, are administered on day 1, 2 or 3 of a 14-days cycle and the second dose of the first and second antibody are administered on day 8, 9 or 10 of a 14 days cycle followed by administration of the first and second antibody, or a pharmaceutical acceptable salt thereof, on day 1 of a 14-days cycle (Q2W). In one embodiment the first and second antibody, or pharmaceutical acceptable salt thereof, are administered on day 1 and day 8 of a 14-days cycle for the first four cycles followed by administration of the first and second antibody, or a pharmaceutical acceptable salt thereof, on day 1 of a 14-days cycle (Q2W).

Hereby, a dosage regimen is provided where the subject to be treated is dosed with an intensified regimen with a weekly dosage for the first 8 weeks of treatment followed by a biweekly dosage regimen (Q2W).

In one embodiment of the present invention, the first and second antibody binding to DR5, or pharmaceutical acceptable salt thereof, is administered twice in a 14-days cycle followed by continued administration on day 1 of a 14-days cycle (Q2W). In one embodiment of the present invention, the first and second antibody binding to DR5, or pharmaceutical acceptable salt thereof, is administered on day 1, 2 or 3 and 8, 9 or 10 of a first 14-days cycle (priming), followed by continued administration on day 1 of a 14-days cycle. In on embodiment of the present invention, the first and second antibody binding to DR5, or pharmaceutical acceptable salt thereof, is administered on day 1 and 8 of a first 14-days cycle (priming), followed by administration on day 1 of a 14-days cycle (Q2W). Thus, for the first 2 weeks the first and second antibody binding to DR5 are administered according to a priming regimen to allow for desensitization of the subjects to the therapy. Thus, the priming dose(s) may reduce potential toxicities of higher doses of treatment. The priming doses used at the initiation of the therapy is a lower dose of the first and second antibody binding to DR5 than the dose administered in the following 14-day cycles. Once the subject has received treatment based on a priming dose the therapy may be based on a biweekly dosage regimen, such as on day 1 of a 14-days cycle.

In one embodiment of the present invention, the first and second antibody binding to DR5, or pharmaceutical acceptable salt thereof, are administered once in a first 14-days cycle as a priming dose followed by continued administration on day 1 of a 14-days cycle (Q2W). In one embodiment of the invention the first and second antibody binding to DR5, or pharmaceutical acceptable salt thereof, are administered on day 1, 2 or 3 of the first 14-days cycles (priming), followed by continued administration on day 1, 2 or 3 of a 14-day cycle. In one embodiment of the invention the first and second antibody binding to DR5, or pharmaceutical acceptable salt thereof, are administered on day 1 of the first 14-day cycle (priming), followed by administration on day 1 of a 14-day cycle (Q2W). Thus, the administration of the first and second antibody binding to DR5 in the first 14-days cycles is the administration according to a priming regimen which allow for desensitization of the subjects to the therapy and reduce potential toxicities of higher doses of treatment. Thus, following the initial priming doses the subject may receive treatment administered based on a biweekly dosage regimen, where the following doses are a higher dose than the priming doses. The priming dose administered is a lower dose of the first and second antibody binding to DR5 than the dose administered in the following 14-day cycles. Thus, the first priming dose may be of 0.1 mg/kg whereas the following doses may be from 0.3 mg/kg to 18 mg/kg. Thus, the priming dose may be a lower dose than the following doses administered to the subject. The priming doses used at the initiation of therapy may be used for desensitization of the subjects to the therapy and thereby the priming dose(s) may reduce potential toxicities of higher doses of treatment.

In one embodiment of the present invention, the first and second antibody binding to DR5, or pharmaceutical acceptable salt thereof, are administered once in a first and second 14-days cycle as a priming dose followed by continued administration on day 1 of a 14-days cycle (Q2W). In one embodiment of the invention the first and second antibody binding to DR5, or pharmaceutical acceptable salt thereof, are administered on day 1, 2 or 3 of the first and second 14-days cycles (priming), followed by continued administration on day 1, 2 or 3 of a 14-day cycle. In one embodiment of the invention the first and second antibody binding to DR5, or pharmaceutical acceptable salt thereof, are administered on day 1 of the first and second 14-days cycles (priming), followed by administration on day 1 of a 14-day cycle (Q2W). Thus, the administration of the first and second antibody binding to DR5 in the first and second 14-days cycles is the administration according to a priming regimen which allow for desensitization of the subjects to the therapy and reduce potential toxicities of higher doses of treatment. Thus, following the initial priming doses the subject may receive treatment administered based on a biweekly dosage regimen, where the following doses are a higher dose than the priming doses. The priming dose administered is a lower dose of the first and second antibody binding to DR5 than the dose administered in the following 14-day cycles. Thus, the first priming dose may be of 1 mg/kg and the second priming dose may be from 1 mg/kg to 6 mg/kg, whereas the following doses may be from 3 mg/kg to 15 mg/kg. Thus, the priming dose may be a lower dose than the following doses administered to the subject. The priming doses used at the initiation of therapy may be used for desensitization of the subjects to the therapy and thereby the priming dose(s) may reduce potential toxicities of higher doses of treatment.

The present invention encompasses embodiments wherein the subject remains on the biweekly (Q2W) treatment cycle, such as on day 1 of a 14-days cycle for at least 2, 3, 4, 5, 6, 7, 8, 9, 10, 11, 12 or more cycles. In another embodiment, the subject remains on the biweekly treatment cycle for between 2 and 48 cycles, such as between 2 and 36 cycles, such as between 2 and 24 cycles, such as between 2 and 15 cycles, such as between 2 and 12 cycles, such as 2 cycles, 3 cycles, 4 cycles, 5 cycles, 6 cycles, 7 cycles, 8 cycles, 9 cycles, 10 cycles, 11 cycles or 12 cycles wherein each cycle is 14 days as described above. In some embodiments, the subject remains on the Q2W treatment cycle for 12 cycles or more, such as 16 cycles or more, such as 24 cycles or more, such as 36 cycles or more. In some embodiments, the first and second antibodies are administered for no more than 3, no more than 4, no more than 5, or no more than 6, no more than 7, no more than 8, no more than 9, no more than 10, no more than 11, no more than 12 14-days treatment cycles. The number of treatment cycles suitable for any specific subject or group of subjects may be determined by a person of skill in the art, typically a physician. For example, such a person may evaluate the response to the anti-DR5 antibody treatment based on the criteria provided in Table 1 (RECIST Criteria v1.1).

In certain embodiments of the invention, the first or second antibody, or a pharmaceutically acceptable salt thereof, is administered at a dose ranging from about 0.05 mg/kg to 9 mg/kg or about 0.15 mg/kg to 18 mg/kg. Thus, the dosage may be adjusted to the subject's body weight. In one embodiment of the invention, the first or second antibody, or a pharmaceutically acceptable salt thereof, is administered at a dose ranging from about 0.05 mg/kg to 6 mg/kg or about 0.15 mg/kg to 9 mg/kg. In one embodiment of the invention, the first or second antibody, or a pharmaceutically acceptable salt thereof, is administered at a dose of about 0.05 mg/kg, or a dose of about 0.15 mg/kg, or a dose of about 0.3 mg/kg, or a dose of about 0.5 mg/kg, or a dose of about 1 mg/kg, or a dose of about 1.5 mg/kg, or a dose of about 2.25 mg/kg, or a dose of about 3 mg/kg, or a dose of about 4.5 mg/kg, or a dose of about 6 mg/kg, or a dose of about 7.5 mg/kg, or a dose of about 9 mg/kg. In one embodiment of the invention, the first or second antibody, or a pharmaceutically acceptable salt thereof, is administered at dose range of about 0.1 mg/kg to 3 mg/kg or about 1 mg/kg to 6 mg/kg. In one embodiment of the invention, the first or second antibody, or a pharmaceutically acceptable salt thereof, is administered at a dose of about 0.05 mg/kg, 0.15 mg/kg, 0.3 mg/kg, 0.5 mg/kg, 1 mg/kg, 1.5 mg/kg, 2.25 mg/kg, 3 mg/kg, 4.5 mg/kg, 6 mg/kg, 7.5 mg/kg or 9 mg/kg.

In some embodiments, the biweekly dose of the first or second antibody will be about 0.05 mg/kg body weight. In some embodiments, the biweekly dose of the first or second antibody will be about 0.15 mg/kg body weight. In some embodiments, the biweekly dose of the first or second antibody will be about 0.3 mg/kg body weight. In some embodiments, the biweekly dose of the first or second antibody will be about 0.5 mg/kg body weight. In some embodiments, the biweekly dose of the first or second antibody will be about 1 mg/kg body weight. In some embodiments, the weekly dose of the first or second antibody will be about 1.5 mg/kg body weight. In some embodiments, the biweekly dose of the first or second antibody will be about 2.25 mg/kg body weight. In some embodiments, the biweekly dose of the first or second antibody will be about 3 mg/kg body weight. In some embodiments, the biweekly dose of the first or second antibody will be about 4.5 mg/kg body weight. In some embodiments, the biweekly dose of the first or second antibody will be about 6 mg/kg body weight. In some embodiments, the biweekly dose of the first or second antibody will be about 7.5 mg/kg body weight. In some embodiments, the biweekly dose of the first or second antibody will be about 9 mg/kg body weight. Hereby biweekly doses are provided where the biweekly dose may be either the priming dose or the dose following the biweekly dose i.e. the treatment dose.

In one embodiment of the invention, the first or second antibody, or a pharmaceutically acceptable salt thereof, is administered to the subject on day 1 of a 7-days cycle for a first 8 weeks at a dose ranging from about 0.15 mg/kg to 9 mg/kg. In one embodiment of the invention, the first or second antibody, or a pharmaceutically acceptable salt thereof, is administered to the subject on day 1 of a 7-days cycle for a first 8 weeks at a dose of about 0.30 mg/kg. In one embodiment of the invention, the first or second antibody, or a pharmaceutically acceptable salt thereof, is administered to the subject on day 1 of a 7-days cycle for a first 8 weeks at a dose of about 0.5 mg/kg. In one embodiment of the invention, the first or second antibody, or a pharmaceutically acceptable salt thereof, is administered to the subject on day 1 of a 7-days cycle for a first 8 weeks at a dose of about 1 mg/kg. In one embodiment of the invention, the first or second antibody, or a pharmaceutically acceptable salt thereof, is administered to the subject on day 1 of a 7-days cycle for a first 8 weeks at a dose of about 1.5 mg/kg. In one embodiment of the invention, the first or second antibody, or a pharmaceutically acceptable salt thereof, is administered to the subject on day 1 of a 7-days cycle for a first 8 weeks at a dose of about 2.25 mg/kg. In one embodiment of the invention, the first or second antibody, or a pharmaceutically acceptable salt thereof, is administered to the subject on day 1 of a 7-days cycle for a first 8 weeks at a dose of about 3 mg/kg. In one embodiment of the invention, the first or second antibody, or a pharmaceutically acceptable salt thereof, is administered to the subject on day 1 of a 7-days cycle for a first 8 weeks at a dose of about 4.5 mg/kg. In one embodiment of the invention, the first or second antibody, or a pharmaceutically acceptable salt thereof, is administered to the subject on day 1 of a 7-days cycle for a first 8 weeks at a dose of about 6 mg/kg. In one embodiment of the invention, the first or second antibody, or a pharmaceutically acceptable salt thereof, is administered to the subject on day 1 of a 7-days cycle for a first 8 weeks at a dose of about 7.5 mg/kg. In one embodiment of the invention, the first or second antibody, or a pharmaceutically acceptable salt thereof, is administered to the subject on day 1 of a 7-days cycle for a first 8 weeks at a dose of about 9 mg/kg.

In one embodiment of the invention, the first or second antibody, or a pharmaceutically acceptable salt thereof, is administered to the subject on day 1 and day 8 of a first 14-days cycle at a dose ranging from about 0.15 mg/kg to 3 mg/kg. In one embodiment of the invention, the first or second antibody, or a pharmaceutically acceptable salt thereof, is administered to the subject on day 1 and day 8 of a first 14-days cycle at a dose of about 0.15 mg/kg. In one embodiment of the invention, the first or second antibody, or a pharmaceutically acceptable salt thereof, is administered to the subject on day 1 and day 8 of a first 14-days cycle at a dose of about 0.30 mg/kg. In one embodiment of the invention, the first or second antibody, or a pharmaceutically acceptable salt thereof, is administered to the subject on day 1 and day 8 of a first 14-days cycle at a dose of about 0.5 mg/kg. In one embodiment of the invention, the first or second antibody, or a pharmaceutically acceptable salt thereof, is administered to the subject on day 1 and day 8 of a first 14-days cycle at a dose of about 1 mg/kg. In one embodiment of the invention, the first or second antibody, or a pharmaceutically acceptable salt thereof, is administered to the subject on day 1 and day 8 of a first 14-days cycle at a dose of about 1.5 mg/kg. In one embodiment of the invention, the first or second antibody, or a pharmaceutically acceptable salt thereof, is administered to the subject on day 1 and day 8 of a first 14-days cycle at a dose of about 2 mg/kg. In one embodiment of the invention, the first or second antibody, or a pharmaceutically acceptable salt thereof, is administered to the subject on day 1 and day 8 of a first 14-days cycle at a dose of about 2.25 mg/kg. In one embodiment of the invention, the first or second antibody, or a pharmaceutically acceptable salt thereof, is administered to the subject on day 1 and day 8 of a first 14-days cycle at a dose of about 3 mg/kg.

In one embodiment of the invention, the first or second antibody, or a pharmaceutically acceptable salt thereof, is administered to the subject on day 1 of a first 14-days cycle at a dose ranging from about 0.05 mg/kg to 0.15 mg/kg. In one embodiment of the invention, the first or second antibody, or a pharmaceutically acceptable salt thereof, is administered to the subject on day 1 of a first 14-days cycle at a dose of 0.05 mg/kg. In one embodiment of the invention, the first or second antibody, or a pharmaceutically acceptable salt thereof, is administered to the subject on day 1 of a first 14-days cycle at a dose of 0.15 mg/kg. In one embodiment of the invention, the first or second antibody, or a pharmaceutically acceptable salt thereof, is administered to the subject on day 1 of a first 14-days cycle at a dose of 0.30 mg/kg. In one embodiment of the invention, the first or second antibody, or a pharmaceutically acceptable salt thereof, is administered to the subject on day 1 of a first 14-days cycle at a dose of 0.5 mg/kg. In one embodiment of the invention, the first or second antibody, or a pharmaceutically acceptable salt thereof, is administered to the subject on day 1 of a first 14-days cycle at a dose of 1 mg/kg. In one embodiment of the invention, the first or second antibody, or a pharmaceutically acceptable salt thereof, is administered to the subject on day 1 of a first 14-days cycle at a dose of 2 mg/kg.

In one embodiment of the invention, the first or second antibody, or a pharmaceutically acceptable salt thereof is administered to the subject on day 1 of a first 14-day cycle at a dose ranging from about 0.05 mg/kg to 1 mg/kg, such as ranging from about 0.05 mg/kg to 0.3 mg/kg.

In one embodiment of the invention, the first or second antibody, or a pharmaceutically acceptable salt thereof, is administered to the subject on day 1 of a first and second 14-days cycle at a dose ranging from about 0.15 mg/kg to 1 mg/kg. In one embodiment of the invention, the first or second antibody, or a pharmaceutically acceptable salt thereof, is administered to the subject on day 1 of a first and second 14-days cycle at a dose ranging from about 0.15 mg/kg to 0.5 mg/kg. In one embodiment of the invention, the first or second antibody, or a pharmaceutically acceptable salt thereof, is administered to the subject on day 1 of a first and second 14-days cycle at a dose of 0.15 mg/kg. In one embodiment of the invention, the first or second antibody, or a pharmaceutically acceptable salt thereof, is administered to the subject on day 1 of a first and second 14-days cycle at a dose of 0.30 mg/kg. In one embodiment of the invention, the first or second antibody, or a pharmaceutically acceptable salt thereof, is administered to the subject on day 1 of a first and second 14-days cycle at a dose of 1 mg/kg. In one embodiment of the invention, the first or second antibody, or a pharmaceutically acceptable salt thereof, is administered to the subject on day 1 of a first and second 14-days cycle at a dose of 1.5 mg/kg. In one embodiment of the invention, the first or second antibody, or a pharmaceutically acceptable salt thereof, is administered to the subject on day 1 of a first and second 14-days cycle at a dose of 2 mg/kg.

In one embodiment of the invention, the first or second antibody, or a pharmaceutically acceptable salt thereof, is administered to the subject on day 8 of a first 14-days cycle at a dose ranging from about 0.5 mg/kg to 3 mg/kg. In one embodiment of the invention, the first or second antibody, or a pharmaceutically acceptable salt thereof, is administered to the subject on day 1 of a first and second 14-days cycle at a dose 0.5 mg/kg. In one embodiment of the invention, the first or second antibody, or a pharmaceutically acceptable salt thereof, is administered to the subject on day 1 of a first and second 14-days cycle at a dose 1 mg/kg. In one embodiment of the invention, the first or second antibody, or a pharmaceutically acceptable salt thereof, is administered to the subject on day 1 of a first and second 14-days cycle at a dose 1.5 mg/kg. In one embodiment of the invention, the first or second antibody, or a pharmaceutically acceptable salt thereof, is administered to the subject on day 1 of a first and second 14-days cycle at a dose 2 mg/kg. In one embodiment of the invention, the first or second antibody, or a pharmaceutically acceptable salt thereof, is administered to the subject on day 1 of a first and second 14-days cycle at a dose 2.25 mg/kg. In one embodiment of the invention, the first or second antibody, or a pharmaceutically acceptable salt thereof, is administered to the subject on day 1 of a first and second 14-days cycle at a dose 3 mg/kg. In one embodiment of the invention, the first or second antibody, or a pharmaceutically acceptable salt thereof, is administered to the subject on day 1 of a first and second 14-days cycle at a dose 3.5 mg/kg. In one embodiment of the invention, the first or second antibody, or a pharmaceutically acceptable salt thereof, is administered to the subject on day 1 of a first and second 14-days cycle at a dose 4 mg/kg.

In one embodiment of the invention, the first and second antibody, or a pharmaceutically acceptable salt thereof, are combined; then the total amount of antibody administered is at a dose ranging from about 0.1 mg/kg to 18 mg/kg or from about 0.3 mg/kg to 18 mg/kg. Thus, in some embodiments, the dose administered is described as the combined amount of a first and second antibody administered to the subject. Thus, in some embodiments where e.g. 1 mg/kg of the first antibody is administered to the subject and 1 mg/kg of the second antibody is administered to the subject, the combined total amount of antibody administered to the subject is a dose of 2 mg/kg.

In one embodiment of the invention, the first and second antibody, or a pharmaceutically acceptable salt thereof, are combined; then the total amount of antibody administered is at a dose of 0.1 mg/kg. In one embodiment of the invention, the first and second antibody, or a pharmaceutically acceptable salt thereof, are combined; then the total amount of antibody administered is at a dose of 0.3 mg/kg. In one embodiment of the invention, the first and second antibody, or a pharmaceutically acceptable salt thereof, are combined; then the total amount of antibody administered is at a dose of 0.5 mg/kg. In one embodiment of the invention, the first and second antibody, or a pharmaceutically acceptable salt thereof, are combined; then the total amount of antibody administered is at a dose of 0.6 mg/kg. In one embodiment of the invention, the first and second antibody, or a pharmaceutically acceptable salt thereof, are combined; then the total amount of antibody administered is at a dose of 1 mg/kg. In one embodiment of the invention, the first and second antibody, or a pharmaceutically acceptable salt thereof, are combined; then the total amount of antibody administered is at a dose of 2 mg/kg. In one embodiment of the invention, the first and second antibody, or a pharmaceutically acceptable salt thereof, are combined; then the total amount of antibody administered is at a dose of 3 mg/kg. In one embodiment of the invention, the first and second antibody, or a pharmaceutically acceptable salt thereof, are combined; then the total amount of antibody administered is at a dose of 4.5 mg/kg. In one embodiment of the invention, the first and second antibody, or a pharmaceutically acceptable salt thereof, are combined; then the total amount of antibody administered is at a dose of 6 mg/kg. In one embodiment of the invention, the first and second antibody, or a pharmaceutically acceptable salt thereof, are combined; then the total amount of antibody administered is at a dose of 8 mg/kg. In one embodiment of the invention, the first and second antibody, or a pharmaceutically acceptable salt thereof, are combined; then the total amount of antibody administered is at a dose of 9 mg/kg. In one embodiment of the invention, the first and second antibody, or a pharmaceutically acceptable salt thereof, are combined; then the total amount of antibody administered is at a dose of 10 mg/kg. In one embodiment of the invention, the first and second antibody, or a pharmaceutically acceptable salt thereof, are combined; then the total amount of antibody administered is at a dose of 12 mg/kg. In one embodiment of the invention, the first and second antibody, or a pharmaceutically acceptable salt thereof, are combined; then the total amount of antibody administered is at a dose of 15 mg/kg. In one embodiment of the invention, the first and second antibody, or a pharmaceutically acceptable salt thereof, are combined; then the total amount of antibody administered is at a dose of 18 mg/kg.

In one embodiment of the invention, the first and second antibody, or a pharmaceutically acceptable salt thereof, are administered at about a 49:1 to 1:49 molar ratio. In one embodiment of the invention, the first and second antibody, or a pharmaceutically acceptable salt thereof, are administered at about a 25:1 to 1:25 molar ratio. In one embodiment of the invention, the first and second antibody, or a pharmaceutically acceptable salt thereof, are administered at about a 15:1 to 1:15 molar ratio. In one embodiment of the invention, the first and second antibody, or a pharmaceutically acceptable salt thereof, are administered at about a 10:1 to 1:10 molar ratio. In one embodiment of the invention, the first and second antibody, or a pharmaceutically acceptable salt thereof, are administered at about a 5:1 to 1:5 molar ratio. In one embodiment of the invention, the first and second antibody, or a pharmaceutically acceptable salt thereof, are administered at about a 2:1 to 1:2 molar ratio. In one embodiment of the invention, the first and second antibody, or a pharmaceutically acceptable salt thereof, are administered at about a 1:1 molar ratio.

In one embodiment of the invention, a steroid hormone is administered to the subject prior to administration of the first and second antibody. In one embodiment of the invention, a steroid hormone is administered from three days prior to seven days after the administration of the first and second antibody. That is in one embodiment the steroid hormone is administered from day −3 to day 8, when the first and second antibody is administered on day 1 of to 14-day cycle. In one embodiment of the invention, a steroid hormone is administered one day to three days prior to the administration of the first and second antibody. In one embodiment of the invention, a steroid hormone is administered one day prior to the administration of the first and second antibody. In one embodiment of the invention, a steroid hormone is administered two days prior to the administration of the first and second antibody. In one embodiment of the invention, a steroid hormone is administered three days prior to the administration of the first and second antibody. In one embodiment of the invention, a steroid hormone is administered to the subject one the same day as the first and second antibody. In one embodiment of the invention, a steroid hormone is administered 1 day to 7 day following the administration of the first and second antibody. In one embodiment of the invention, a steroid hormone is administered 1 day to three days following the administration of the first and second antibody. The effect of administering the steroid hormone is to mitigate potential transaminase elevations caused by administration of the first and second antibody binding to DR5. Thus, administering a steroid may reduce, prevent or lessen the induction of transaminase levels by the first and second antibody, such as reduce, prevent or lessen the induction of alanine transaminase (ALT) or aspartate transaminase (AST).

In one embodiment of the invention the steroid hormone is a corticosteroid. In one embodiment the steroid hormone is dexamethasone.

In one embodiment of the invention, dexamethasone is administered to the subject from three days prior to 7 days after the administration of the first and second antibody. In one embodiment of the invention, dexamethasone is administered to the subject prior to administration of the first and second antibody. In one embodiment of the invention, dexamethasone is administered between one day to three days prior to the administration of the first and second antibody. In one embodiment of the invention, dexamethasone is administered one day prior to the administration of the first and second antibody. In one embodiment of the invention, dexamethasone is administered two days prior to the administration of the first and second antibody. In one embodiment of the invention, dexamethasone is administered three days prior to the administration of the first and second antibody. In one embodiment of the invention, dexamethasone is administered one the day of administration of the first and second antibody.

In one embodiment of the invention, dexamethasone is administered at a dose ranging from 1 to 100 mg. In one embodiment of the invention, dexamethasone is administered at a dose ranging from 5 to 20 mg. Thus, the dexamethasone is administered at a flat dose to the subject which does not depend on the weight of the subject. In one embodiment of the invention, dexamethasone is administered at a dose of 10 mg. Thus, in one embodiment of the invention, dexamethasone is administered at a dose of 10 mg per subject, where the dose administered does not depend on the weight of the subject. In one embodiment of the invention, dexamethasone is administered daily.

In one embodiment of the invention, dexamethasone is administered by intravenous infusion. In one embodiment of the invention, 10 mg dexamethasone is administered by intravenous infusion 1 day prior to the administration of the first and second antibody. Hereby embodiments are described wherein the dexamethasone is administered to mitigate transaminase elevations caused by administration of the first and second antibody binding to DR5. Thus, administering dexamethasone may reduce, prevent or lessen the induction of transaminase levels by the first and second antibody, such as reduce, prevent or lessen the induction of alanine transaminase (ALT) or aspartate transaminase (AST).

### Maintenance Therapy

A person of skill in the art, such as a physician, may determine that, after a suitable number of treatment cycles, the treatment cycles should be followed by maintenance therapy with a first and a second antibody binding to DR5, treatment with another therapeutic agent or combination of therapeutic agents, as appropriate.

In some embodiments, the subject will begin maintenance therapy following one or more, preferably two or more, such as following 3 or 4 or 5 or 6 or 7 or 8 or 9 or 10 or 11 or 12 or more cycles, such as 24 cycles or more, such as 36 cycles or more, of 14 days treatment cycles (Q2W).

In some embodiments, the subject will start maintenance therapy following an evaluation indicating that the subject has reduced amount of cancer or no detectable cancer, e.g., following an evaluation indicating that the subject has had a complete response.

As used herein, “reduced administration frequency refers to therapy with the first and second antibody binding DR5, but at a reduced administration schedule compared to an intensified dosing schedule where the ab is dosed at e.g. once a week. During reduced administration frequency, the first and second antibody binding DR5 is preferably administered once every two weeks (Q2W).

The first and second antibody binding to DR5 may alternatively be administered as a combination therapy. By the term “combination therapy” is meant that at least one other anti-cancer agent is administered to the subject during the treatment cycle with a first and second antibody binding to DR5. The first and second antibody binding to DR5 and the at least one other anti-cancer agent may be administered simultaneously, and may optionally be provided in the same pharmaceutical composition.

Typically, however, the first and second antibody binding to DR5 and the at least one other anti-cancer agent are separately administered and formulated as separate pharmaceutical compositions. For example, the at least one other anti-cancer agent may be administered according to the dosage regimen for which it has been approved by a medicines regulatory authority when administered as a monotherapy, or the at least one other anti-cancer agent may be administered according to a dosage regimen which is optimized for its combined use with the first and second antibody binding to DR5 as described herein.

The response to the anti-DR5 therapy may be evaluated by a person of skill in the art according to known methods, e.g., the guidelines of the NCCN or ESMO. In a specific embodiment, the evaluation can be based on the following criteria (RECIST Criteria v1.1):

TABLE 1
Definition of Response (RECIST Criteria v1.1)

| | Category | Criteria |
|---|---|---|
| Based on target | Complete Response | Disappearance of all target lesions. Any pathological |
| lesions | (CR) | lymph nodes must have reduction in short axis to <10 mm. |
| | Partial Response | ≥30% decrease in the sum of the LD of target lesions, |
| | (PR) | taking as reference the baseline sum LDs. |
| | Stable Disease | Neither sufficient shrinkage to qualify for PR nor sufficient |
| | (SD) | increase to qualify for PD, taking as reference the smallest |
| | | sum of LDs while in trail since the treatment started. |
| | Progressive Disease | ≥20% (and ≥5 mm) increase in the sum of the LDs of |
| | (PD) | target lesions, taking as reference the smallest sum of the |
| | | target LDs recorded while in trail or the appearance of 1 |
| | | or more new lesions. since the treatment started or the |
| | | appearance of one or more new lesions. |
| | NE | Not evaluable |
| | CR | Disappearance of all non-target lesions and normalization |
| | | of tumor marker level. All lymph nodes must be non- |
| | | pathological in size (<10 mm short axis). |
| Based on non- | SD | Persistence of one or more non-target lesion(s) or/and |
| target lesions | | maintenance of tumor marker level above the normal |
| | | limits. |
| | PD | Appearance of one or more new lesions and/or |
| | | unequivocal progression of existing non-target lesions. |
| | NE | Not evaluable |

### Pharmaceutical Compositions

In another aspect of the invention, the first and/or second antibody binding DR5 for use according to any aspect or embodiment of the invention as described herein is comprised in a pharmaceutical composition. In one embodiment the pharmaceutical composition further comprises a pharmaceutically acceptable carrier. In particular, upon purifying the first and/or second antibody binding DR5s they may be formulated into pharmaceutical compositions using well known pharmaceutical carriers or excipients.

The pharmaceutical compositions may be formulated with pharmaceutically acceptable carriers or diluents as well as any known adjuvants and excipients in accordance with conventional techniques such as those disclosed in Remington: The Science and Practice of Pharmacy, 19th Edition, Gennaro, Ed., Mack Publishing Co., Easton, Pa., 1995.

The pharmaceutically acceptable carriers or diluents as well as any known adjuvants and excipients should be suitable for the antibodies of the present invention and the chosen mode of administration. Suitability for carriers and other components of pharmaceutical compositions is determined based on the lack of significant negative effect on the desired biological properties of the compound or pharmaceutical composition of the present invention (e.g., less than a substantial effect (10% or less relative inhibition, 5% or less relative inhibition, etc.)) on antigen binding.

A pharmaceutical composition of the present invention may also include diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition.

The pharmaceutical composition may be administered by any suitable route and mode. Suitable routes of administering an antibody of the present invention are well-known in the art and may be selected by those of ordinary skill in the art.

In one embodiment, the pharmaceutical composition of the present invention is administered by intravenous administration.

In one embodiment, the pharmaceutical composition of the present invention is administered by intravenous infusion.

Pharmaceutically acceptable carriers include any and all suitable solvents, dispersion media, coatings, antibacterial and antifungal agents, isotonicity agents, antioxidants and absorption delaying agents, and the like that are physiologically compatible with the antibodies of the present invention.

Examples of suitable aqueous- and non-aqueous carriers which may be employed in the pharmaceutical compositions of the present invention include water, saline, phosphate-buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the first and/or second antibody of the present invention, use thereof in the pharmaceutical compositions of the present invention is contemplated.

Proper fluidity may be maintained, for example, by the use of coating materials, such as lecithin, by the maintenance of the required particle size in the case of dispersions, and by the use of surfactants.

The pharmaceutical compositions of the present invention may also comprise pharmaceutically acceptable antioxidants for instance (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present invention may also comprise isotonicity agents, such as sugars, polyalcohols, such as mannitol, sorbitol, glycerol or sodium chloride.

The pharmaceutical compositions of the present invention may also contain one or more adjuvants appropriate for the chosen route of administration such as preservatives, wetting agents, emulsifying agents, dispersing agents or buffers, which may prolong the shelf life or effectiveness of the pharmaceutical composition. The first and/or second antibody binding DR5 of the present invention may be prepared with carriers that will protect the compound against rapid release, such as a controlled release formulations, including implants, transdermal patches, and microencapsulated delivery systems.

Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well known in the art. Methods for the preparation of such formulations are generally known to those skilled in the art. See e.g., Sustained and Controlled Release Drug Delivery Systems, J. R. Robinson, ed., Marcel Dekker, Inc., New York, 1978.

In one embodiment, the first and/or second antibody binding DR5 of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated. Supplementary active compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or nonaqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the first and/or second antibody binding DR5 in the required amount in an appropriate solvent with one or a combination of ingredients e.g. as enumerated above, as required, followed by sterilization microfiltration. Generally, dispersions are prepared by incorporating the first and/or second antibody binding DR5 into a sterile vehicle that contains a basic dispersion medium and the required other ingredients e.g. from those enumerated above.

Sterile injectable solutions may be prepared by incorporating the first and/or second antibody binding DR5 in the required amount in an appropriate solvent with one or a combination of ingredients enumerated above, as required, followed by sterilization microfiltration. Generally, dispersions are prepared by incorporating the first and/or second antibody binding DR5 into a sterile vehicle that contains a basic dispersion medium and the required other ingredients from those enumerated above.

In one particular embodiment, the first and/or second antibody binding DR5 is comprised in a pharmaceutical composition which comprises one or more excipients but is free of surfactant. In one embodiment, the pharmaceutical composition has a pH of about 5.5 to about 7 and comprises, in aqueous solution:
(a) from about 5 mg/mL to about 30 mg/mL of a first and second antibody binding to DR5;
(b) histidine; and
(c) sodium chloride.

In one embodiment of the invention, the pharmaceutical composition has a pH of about 6.

In a specific embodiment, the pharmaceutical composition has a pH in the range of about 5.5 to about 6.5 and comprises:

(a) from about 2 mg/mL to about 20 mg/mL of a first and second antibody binding DR5, such as about 10 mg/mL of the first antibody binding DR5 and 10 mg/mL of the second antibody binding DR5;

(b) from about 10 mM to about 50 mM histidine, such as about 30 mM histidine;

(c) from about 50 mM to 250 mM sodium chloride, such as about 150 mM sodium chloride.

In one embodiment of the invention, the pharmaceutical composition has a pH of about 6 and comprises:
(a) 10 mg/mL of the first antibody binding DR5 and 10 mg/mL of the second antibody binding DR5;
(b) from about 30 mM histidine; and
(c) 150 mM sodium chloride.

In one embodiment of the invention, the pharmaceutical composition has a pH of about 6 and comprises:
(a) 20 mg/mL of the first antibody binding DR5 and 20 mg/mL of the second antibody binding DR5;
(b) from about 30 mM histidine; and
(c) 150 mM sodium chloride.

TABLE 2
Sequences

| SEQ ID | | | |
|---|---|---|---|
| NO: | Name | Sequence | Remarks |
| SEQ ID | VH hDR5-01 | GFNIKDTF | Clone IgG1- |
| NO: 1 | CDR1 | | hDR5-01 |
| SEQ ID | VH hDR5-01 | IDPANGNT | |
| NO: 2 | CDR2 | | |
| SEQ ID | VH hDR5-01 | VRGLYTYYFDY | |
| NO: 3 | CDR3 | | |
| SEQ ID | VH hDR5-01 | EVQLQQSGAEVVKPGASVKLSCKASGFNIKDTFIHWVKQAPGQ | |
| NO: 4 | | GLEWIGRIDPANGNTKYDPKFQGKATITTDTSSNTAYMELSSLRS | |
| | | EDTAVYYCVRGLYTYYFDYWGQGTLVTVSS | |
| SEQ ID | VL hDR5-01 | QSISNN | |
| NO: 5 | CDR1 | | |
| | VL hDR5-01 | FAS | |
| | CDR2 | | |
| SEQ ID | VL hDR5-01 | QQGNSWPYT | |
| NO: 6 | CDR3 | | |
| SEQ ID | VL hDR5-01 | EIVMTQSPATLSVSPGERATLSCRASQSISNNLHWYQQKPGQAP | |
| NO: 7 | | RLLIKFASQSITGIPARFSGSGSGTEFTLTISSLQSEDFAVYYCQQG | |
| | | NSWPYTFGQGTKLEIK | |
| SEQ ID | VH hDR5-01- | GFNIKDTF | Clone IgG1- |
| NO: 1 | G56T CDR1 | | hhDR5-01-G56T |
| SEQ ID | VH hDR5-01- | IDPANTNT | |
| NO: 8 | G56T CDR2 | | |
| SEQ ID | VH hDR5-01- | VRGLYTYYFDY | |
| NO: 3 | G56T CDR3 | | |
| SEQ ID | VH hDR5-01- | EVQLQQSGAEVVKPGASVKLSCKASGFNIKDTFIHWVKQAPGQ | |
| NO: 9 | G56T | GLEWIGRIDPANTNTKYDPKFQGKATITTDTSSNTAYMELSSLRS | |
| | | EDTAVYYCVRGLYTYYFDYWGQGTLVTVSS | |
| SEQ ID | VL hDR5-01- | QSISNN | |
| NO: 5 | G56T CDR1 | | |
| | VL hDR5-01- | FAS | |
| | G56T CDR2 | | |
| SEQ ID | VL hDR5-01- | QQGNSWPYT | |
| NO: 6 | G56T CDR3 | | |
| SEQ ID | VL hDR5-01- | EIVMTQSPATLSVSPGERATLSCRASQSISNNLHWYQQKPGQAP | |
| NO: 7 | G56T | RLLIKFASQSITGIPARFSGSGSGTEFTLTISSLQSEDFAVYYCQQG | |
| | | NSWPYTFGQGTKLEIK | |
| SEQ ID | VH hDR5-05 | GFNIKDTH | Clone IgG1- |
| NO: | CDR1 | | hDR5-05 |
| 10 | | | |
| SEQ ID | VH hDR5-05 | IDPANGNT | |
| NO: 2 | CDR2 | | |
| SEQ ID | VH hDR5-05 | ARWGTNVYFAY | |
| NO: | CDR3 | | |
| 11 | | | |
| SEQ ID | VH hDR5-05 | QVQLVQSGAEVKKPGASVKVSCKASGFNIKDTHMHWVRQAPG | |
| NO: | | QRLEWIGRIDPANGNTEYDQKFQGRVTITVDTSASTAYMELSSL | |
| 12 | | RSEDTAVYYCARWGTNVYFAYWGQGTLVTVSS | |
| SEQ ID | VL hDR5-05 | SSVSY | |
| NO: 13 | CDR1 | | |
| | VL hDR5-05 | RTS | |
| | CDR2 | | |
| SEQ ID | VL hDR5-05 | QQYHSYPPT | |
| NO: | CDR3 | | |
| 14 | | | |
| SEQ ID | VL hDR5-05 | DIQLTQSPSSLSASVGDRVTITCSASSSVSYMYWYQQKPGKAPKP | |
| NO: | | WIYRTSNLASGVPSRFSGSGSGTDFTLTISSLQPEDFATYYCQQY | |
| 15 | | HSYPPTFGGGTKVEIK | |
| SEQ ID | VH CONA-CDR1 | GGSISSGDYF | Clone |
| NO: | | | Conatumumab |
| 16 | | | IgG1-DR5-CONA |
| SEQ ID | VH CONA-CDR2 | IHNSGTT | |
| NO: | | | |
| 17 | | | |
| SEQ ID | VH CONA-CDR3 | ARDRGGDYYYGMDV | |
| NO: | | | |
| 18 | | | |
| SEQ ID | VH CONA | QVQLQESGPGLVKPSQTLSLTCTVSGGSISSGDYFWSWIRQLPG | |
| NO: | | KGLECIGHIHNSGTTYYNPSLKSRVTISVDTSKKQFSLRLSSVTAAD | |
| 19 | | TAVYYCARDRGGDYYYGMDVWGQGTTVTVSS | |
| SEQ ID | VH CONA-C49W | QVQLQESGPGLVKPSQTLSLTCTVSGGSISSGDYFWSWIRQLPG | Conatumumab |
| NO: | | KGLEWIGHIHNSGTTYYNPSLKSRVTISVDTSKKQFSLRLSSVTAA | IgG1-DRS- |
| 20 | | DTAVYYCARDRGGDYYYGMDVWGQGTTVTVSS | CONA with |
| | | | removal of a |
| | | | cysteine |
| | | | residue |
| | | | (C49W) |
| SEQ ID | VL CONA-CDR1 | QGISRSY | The antibodies |
| NO: | | | IgG1-DRS- |
| 21 | | | CONA and |
| | | | IgG1-DR5- |
| | | | CONA-C49W |
| | | | share the |
| | | | same VL |
| | | | sequence |
| | VL CONA-CDR2 | GAS | |
| SEQ ID | VL CONA-CDR3 | QQFGSSPWT | |
| NO: | | | |
| 22 | | | |
| SEQ ID | VL CONA | EIVLTQSPGTLSLSPGERATLSCRASQGISRSYLAWYQQKPGQAP | |
| NO: | | SLLIYGASSRATGIPDRFSGSGSGTDFTLTISRLEPEDFAVYYCQQF | |
| 23 | | GSSPWTFGQGTKVEIK | |
| SEQ ID | Human DR5 | MEQRGQNAPAASGARKRHGPGPREARGARPGPRVPKTLVLVV | Human DR5 |
| NO: 24 | (Uniprot | AAVLLLVSAESALITQQDLAPQQRAAPQQKRSSPSEGLCPPGHHI | isoform long |
| | O14763-1) | SEDGRDCISCKYGQDYSTHWNDLLFCLRCTRCDSGEVELSPCTTT | |
| | | RNTVCQCEEGTFREEDSPEMCRKCRTGCPRGMVKVGDCTPWS | |
| | | DIECVHKESGTKHSGEVPAVEETVTSSPGTPASPCSLSGIIIGVTVA | |
| | | AVVLIVAVFVCKSLLWKKVLPYLKGICSGGGGDPERVDRSSQRPG | |
| | | AEDNVLNEIVSILQPTQVPEQEMEVQEPAEPTGVNMLSPGESEH | |
| | | LLEPAEAERSQRRRLLVPANEGDPTETLRQCFDDFADLVPFDSW | |
| | | EPLMRKLGLMDNEIKVAKAEAAGHRDTLYTMLIKWVNKTGRDA | |
| | | SVHTLLDALETLGERLAKQKIEDHLLSSGKFMYLEGNADSAMS | |
| SEQ ID | HC hDR5-01 | EVQLQQSGAEVVKPGASVKLSCKASGFNIKDTFIHWVKQAPGQ | Clone IgG1- |
| NO: 25 | | GLEWIGRIDPANGNTKYDPKFQGKATITTDTSSNTAYMELSSLRS | hDR5-01 |
| | | EDTAVYYCVRGLYTYYFDYWGQGTLVTVSSASTKGPSVFPLAPSS | |
| | | KSTSGGTAALGCLVKDYFPEPVTVSWNSGALTSGVHTFPAVLQS | |
| | | SGLYSLSSVVTVPSSSLGTQTYICNVNHKPSNTKVDKRVEPKSCD | |
| | | KTHTCPPCPAPELLGGPSVFLFPPKPKDTLMISRTPEVTCVVVDV | |
| | | SHEDPEVKFNWYVDGVEVHNAKTKPREEQYNSTYRVVSVLTVL | |
| | | HQDWLNGKEYKCKVSNKALPAPIEKTISKAKGQPREPQVYTLPP | |
| | | SREEMTKNQVSLTCLVKGFYPSDIAVEWESNGQPENNYKTTPPV | |
| | | LDSDGSFFLYSKLTVDKSRWQQGNVFSCSVMHEALHNHYTQKS | |
| | | LSLSPGK | |
| SEQ ID | HC hDR5-01- | EVQLQQSGAEVVKPGASVKLSCKASGFNIKDTFIHWVKQAPGQ | Clone IgG1- |
| NO: 26 | K409R-E430G | GLEWIGRIDPANGNTKYDPKFQGKATITTDTSSNTAYMELSSLRS | hDR5-01- |
| | | EDTAVYYCVRGLYTYYFDYWGQGTLVTVSSASTKGPSVFPLAPSS | K409R-E430G |
| | | KSTSGGTAALGCLVKDYFPEPVTVSWNSGALTSGVHTEPAVLQS | |
| | | SGLYSLSSVVTVPSSSLGTQTYICNVNHKPSNTKVDKRVEPKSCD | |
| | | KTHTCPPCPAPELLGGPSVFLFPPKPKDTLMISRTPEVTCVVVDV | |
| | | SHEDPEVKFNWYVDGVEVHNAKTKPREEQYNSTYRVVSVLTVL | |
| | | HQDWLNGKEYKCKVSNKALPAPIEKTISKAKGQPREPQVYTLPP | |
| | | SREEMTKNQVSLTCLVKGFYPSDIAVEWESNGQPENNYKTTPPV | |
| | | LDSDGSFFLYSRLTVDKSRWQQGNVFSCSVMHGALHNHYTQKS | |
| | | LSLSPGK | |
| SEQ ID | LC hDR5-01 | EIVMTQSPATLSVSPGERATLSCRASQSISNNLHWYQQKPGQAP | Clone IgG1- |
| NO: 27 | | RLLIKFASQSITGIPARFSGSGSGTEFTLTISSLQSEDFAVYYCQQG | hDR5-01 |
| | | NSWPYTFGQGTKLEIKRTVAAPSVFIFPPSDEQLKSGTASVVCLL | |
| | | NNFYPREAKVQWKVDNALQSGNSQESVTEQDSKDSTYSLSSTLT | |
| | | LSKADYEKHKVYACEVTHQGLSSPVTKSFNRGEC | |
| SEQ ID | HC hDR5-01- | EVQLQQSGAEVVKPGASVKLSCKASGFNIKDTFIHWVKQAPGQ | Clone IgG1- |
| No:28 | F405L | GLEWIGRIDPANTNTKYDPKFQGKATITTDTSSNTAYMELSSLRS | hDR5-01-F405L |
| | | EDTAVYYCVRGLYTYYFDYWGQGTLVTVSSASTKGPSVFPLAPSS | |
| | | KSTSGGTAALGCLVKDYFPEPVTVSWNSGALTSGVHTEPAVLQS | |
| | | SGLYSLSSVVTVPSSSLGTQTYICNVNHKPSNTKVDKRVEPKSCD | |
| | | KTHTCPPCPAPELLGGPSVFLFPPKPKDTLMISRTPEVTCVVVDV | |
| | | SHEDPEVKFNWYVDGVEVHNAKTKPREEQYNSTYRVVSVLTVL | |
| | | HQDWLNGKEYKCKVSNKALPAPIEKTISKAKGQPREPQVYTLPP | |
| | | SREEMTKNQVSLTCLVKGFYPSDIAVEWESNGQPENNYKTTPPV | |
| | | LDSDGSFLLYSKLTVDKSRWQQGNVFSCSVMHEALHNHYTQKS | |
| | | LSLSPGK | |
| SEQ ID | HC hDR5-01- | EVQLQQSGAEVVKPGASVKLSCKASGFNIKDTFIHWVKQAPGQ | Clone IgG1- |
| NO: 29 | G56T | GLEWIGRIDPANTNTKYDPKFQGKATITTDTSSNTAYMELSSLRS | hDR5-01-G56T |
| | | EDTAVYYCVRGLYTYYFDYWGQGTLVTVSSASTKGPSVFPLAPSS | |
| | | KSTSGGTAALGCLVKDYFPEPVTVSWNSGALTSGVHTFPAVLQS | |
| | | SGLYSLSSVVTVPSSSLGTQTYICNVNHKPSNTKVDKRVEPKSCD | |
| | | KTHTCPPCPAPELLGGPSVFLFPPKPKDTLMISRTPEVTCVVVDV | |
| | | SHEDPEVKFNWYVDGVEVHNAKTKPREEQYNSTYRVVSVLTVL | |
| | | HQDWLNGKEYKCKVSNKALPAPIEKTISKAKGQPREPQVYTLPP | |
| | | SREEMTKNQVSLTCLVKGFYPSDIAVEWESNGQPENNYKTTPPV | |
| | | LDSDGSFFLYSKLTVDKSRWQQGNVFSCSVMHEALHNHYTQKS | |
| | | LSLSPGK | |
| SEQ ID | HC hDR5-01- | EVQLQQSGAEVVKPGASVKLSCKASGFNIKDTFIHWVKQAPGQ | Clone IgG1- |
| NO: 30 | G56T-E430G | GLEWIGRIDPANTNTKYDPKFQGKATITTDTSSNTAYMELSSLRS | hDR5-01- |
| | | EDTAVYYCVRGLYTYYFDYWGQGTLVTVSSASTKGPSVFPLAPSS | G56T-E430G |
| | | KSTSGGTAALGCLVKDYFPEPVTVSWNSGALTSGVHTFPAVLQS | |
| | | SGLYSLSSVVTVPSSSLGTQTYICNVNHKPSNTKVDKRVEPKSCD | |
| | | KTHTCPPCPAPELLGGPSVFLFPPKPKDTLMISRTPEVTCVVVDV | |
| | | SHEDPEVKFNWYVDGVEVHNAKTKPREEQYNSTYRVVSVLTVL | |
| | | HQDWLNGKEYKCKVSNKALPAPIEKTISKAKGQPREPQVYTLPP | |
| | | SREEMTKNQVSLTCLVKGFYPSDIAVEWESNGQPENNYKTTPPV | |
| | | LDSDGSFFLYSKLTVDKSRWQQGNVFSCSVMHGALHNHYTQKS | |
| | | LSLSPGK | |
| SEQ ID | LC hDR5-01- | EIVMTQSPATLSVSPGERATLSCRASQSISNNLHWYQQKPGQAP | Clone IgG1- |
| NO: 27 | G56T | RLLIKFASQSITGIPARFSGSGSGTEFTLTISSLQSEDFAVYYCQQG | hDR5-01-G56T |
| | | NSWPYTFGQGTKLEIKRTVAAPSVFIFPPSDEQLKSGTASVVCLL | |
| | | NNFYPREAKVQWKVDNALQSGNSQESVTEQDSKDSTYSLSSTLT | |
| | | LSKADYEKHKVYACEVTHQGLSSPVTKSFNRGEC | |
| SEQ ID | HC hDR5-05 | QVQLVQSGAEVKKPGASVKVSCKASGFNIKDTHMHWVRQAPG | Clone IgG1- |
| NO: 31 | | QRLEWIGRIDPANGNTEYDQKFQGRVTITVDTSASTAYMELSSL | hDR5-05 |
| | | RSEDTAVYYCARWGTNVYFAYWGQGTLVTVSSASTKGPSVFPL | |
| | | APSSKSTSGGTAALGCLVKDYFPEPVTVSWNSGALTSGVHTFPA | |
| | | VLQSSGLYSLSSVVTVPSSSLGTQTYICNVNHKPSNTKVDKRVEP | |
| | | KSCDKTHTCPPCPAPELLGGPSVFLFPPKPKDTLMISRTPEVTCVV | |
| | | VDVSHEDPEVKFNWYVDGVEVHNAKTKPREEQYNSTYRVVSVL | |
| | | TVLHQDWLNGKEYKCKVSNKALPAPIEKTISKAKGQPREPQVYTL | |
| | | PPSREEMTKNQVSLTCLVKGFYPSDIAVEWESNGQPENNYKTTP | |
| | | PVLDSDGSFFLYSKLTVDKSRWQQGNVFSCSVMHEALHNHYTQ | |
| | | KSLSLSPGK | |
| SEQ ID | HC hDR5-05- | QVQLVQSGAEVKKPGASVKVSCKASGFNIKDTHMHWVRQAPG | Clone IgG1- |
| NO: 32 | E430G | QRLEWIGRIDPANGNTEYDQKFQGRVTITVDTSASTAYMELSSL | hDR5-05-E430G |
| | | RSEDTAVYYCARWGTNVYFAYWGQGTLVTVSSASTKGPSVFPL | |
| | | APSSKSTSGGTAALGCLVKDYFPEPVTVSWNSGALTSGVHTFPA | |
| | | VLQSSGLYSLSSVVTVPSSSLGTQTYICNVNHKPSNTKVDKRVEP | |
| | | KSCDKTHTCPPCPAPELLGGPSVFLFPPKPKDTLMISRTPEVTCVV | |
| | | VDVSHEDPEVKFNWYVDGVEVHNAKTKPREEQYNSTYRVVSVL | |
| | | TVLHQDWLNGKEYKCKVSNKALPAPIEKTISKAKGQPREPQVYTL | |
| | | PPSREEMTKNQVSLTCLVKGFYPSDIAVEWESNGQPENNYKTTP | |
| | | PVLDSDGSFFLYSKLTVDKSRWQQGNVFSCSVMHGALHNHYTQ | |
| | | KSLSLSPGK | |
| SEQ ID | HC hDR5-05- | QVQLVQSGAEVKKPGASVKVSCKASGFNIKDTHMHWVRQAPG | Clone IgG1- |
| NO: 33 | F405L | QRLEWIGRIDPANGNTEYDQKFQGRVTITVDTSASTAYMELSSL | hDR5-05-F405L |
| | | RSEDTAVYYCARWGTNVYFAYWGQGTLVTVSSASTKGPSVFPL | |
| | | APSSKSTSGGTAALGCLVKDYFPEPVTVSWNSGALTSGVHTFPA | |
| | | VLQSSGLYSLSSVVTVPSSSLGTQTYICNVNHKPSNTKVDKRVEP | |
| | | KSCDKTHTCPPCPAPELLGGPSVFLFPPKPKDTLMISRTPEVTCVV | |
| | | VDVSHEDPEVKFNWYVDGVEVHNAKTKPREEQYNSTYRVVSVL | |
| | | TVLHQDWLNGKEYKCKVSNKALPAPIEKTISKAKGQPREPQVYTL | |
| | | PPSREEMTKNQVSLTCLVKGFYPSDIAVEWESNGQPENNYKTTP | |
| | | PVLDSDGSFLLYSKLTVDKSRWQQGNVFSCSVMHEALHNHYTQ | |
| | | KSLSLSPGK | |
| SEQ ID | HC hDR5-05- | QVQLVQSGAEVKKPGASVKVSCKASGFNIKDTHMHWVRQAPG | Clone IgG1- |
| NO: 34 | F405L-E430G | QRLEWIGRIDPANGNTEYDQKFQGRVTITVDTSASTAYMELSSL | hDR5-05- |
| | | RSEDTAVYYCARWGTNVYFAYWGQGTLVTVSSASTKGPSVFPL | F405L-E430G |
| | | APSSKSTSGGTAALGCLVKDYFPEPVTVSWNSGALTSGVHTFPA | |
| | | VLQSSGLYSLSSVVTVPSSSLGTQTYICNVNHKPSNTKVDKRVEP | |
| | | KSCDKTHTCPPCPAPELLGGPSVFLFPPKPKDTLMISRTPEVTCVV | |
| | | VDVSHEDPEVKFNWYVDGVEVHNAKTKPREEQYNSTYRVVSVL | |
| | | TVLHQDWLNGKEYKCKVSNKALPAPIEKTISKAKGQPREPQVYTL | |
| | | PPSREEMTKNQVSLTCLVKGFYPSDIAVEWESNGQPENNYKTTP | |
| | | PVLDSDGSFLLYSKLTVDKSRWQQGNVFSCSVMHGALHNHYTQ | |
| | | KSLSLSPGK | |
| SEQ ID | LC hDR5-05 | DIQLTQSPSSLSASVGDRVTITCSASSSVSYMYWYQQKPGKAPKP | Clone IgG1- |
| NO: 35 | | WIYRTSNLASGVPSRFSGSGSGTDFTLTISSLQPEDFATYYCQQY | hDR5-05 |
| | | HSYPPTFGGGTKVEIKRTVAAPSVFIFPPSDEQLKSGTASVVCLLN | |
| | | NFYPREAKVQWKVDNALQSGNSQESVTEQDSKDSTYSLSSTLTL | |
| | | SKADYEKHKVYACEVTHQGLSSPVTKSFNRGEC | |
| SEQ ID | Human DR5del- | MEQRGQNAPAASGARKRHGPGPREARGARPGPRVPKTLVLVV | Human DR5 |
| NO: 36 | K386N | AAVLLLVSAESALITQQDLAPQQRAAPQQKRSSPSEGLCPPGHHI | (based on |
| | | SEDGRDCISCKYGQDYSTHWNDLLFCLRCTRCDSGEVELSPCTTT | Uniprot |
| | | RNTVCQCEEGTFREEDSPEMCRKCRTGCPRGMVKVGDCTPWS | 014763-2) |
| | | DIECVHKESGIIIGVTVAAVVLIVAVFVCKSLLWKKVLPYLKGICSG | with K386N |
| | | GGGDPERVDRSSQRPGAEDNVLNEIVSILQPTQVPEQEMEVQE | mutation as |
| | | PAEPTGVNMLSPGESEHLLEPAEAERSQRRRLLVPANEGDPTET | used in the |
| | | LRQCFDDFADLVPFDSWEPLMRKLGLMDNEIKVAKAEAAGHR | examples |
| | | DTLYTMLIKWVNKTGRDASVHTLLDALETLGERLANQKIEDHLLS | |
| | | SGKFMYLEGNADSAMS | |
| SEQ ID | Human DR5 | MEQRGQNAPAASGARKRHGPGPREARGARPGPRVPKTLVLVV | Human DR5 |
| NO: 37 | (Uniprot | AAVLLLVSAESALITQQDLAPQQRAAPQQKRSSPSEGLCPPGHHI | isoform short: |
| | 014763-2) | SEDGRDCISCKYGQDYSTHWNDLLFCLRCTRCDSGEVELSPCTTT | missing aa |
| | | RNTVCQCEEGTFREEDSPEMCRKCRTGCPRGMVKVGDCTPWS | 185-213 |
| | | DIECVHKESGIIIGVTVAAVVLIVAVFVCKSLLWKKVLPYLKGICSG | compared to |
| | | GGGDPERVDRSSQRPGAEDNVLNEIVSILQPTQVPEQEMEVQE | Uniprot |
| | | PAEPTGVNMLSPGESEHLLEPAEAERSQRRRLLVPANEGDPTET | 014763-1 in |
| | | LRQCFDDFADLVPFDSWEPLMRKLGLMDNEIKVAKAEAAGHR | SEQ ID NO 24 |
| | | DTLYTMLIKWVNKTGRDASVHTLLDALETLGERLAKQKIEDHLLS | |
| | | SGKFMYLEGNADSAMS | |
| SEQ ID | Human DR5 | MEQRGQNAPAASGARKRHGPGPREARGARPGLRVPKTLVLVV | SEQ ID NO 24 |
| NO: 38 | natural variant | AAVLLLVSAESALITQQDLAPQQRVAPQQKRSSPSEGLCPPGHHI | with naturally |
| | (Accession: | SEDGRDCISCKYGQDYSTHWNDLLFCLRCTRCDSGEVELSPCTTT | occurring |
| | AAB70578) | RNTVCQCEEGTFREEDSPEMCRKCRTGCPRGMVKVGDCTPWS | P32L; A67V; |
| | | DIECVHKESGTKHSGEAPAVEETVTSSPGTPASPCSLSGIIIGVTVA | V191A |
| | | AVVLIVAVFVCKSLLWKKVLPYLKGICSGGGGDPERVDRSSQRPG | substitutions |
| | | AEDNVLNEIVSILQPTQVPEQEMEVQEPAEPTGVNMLSPGESEH | |
| | | LLEPAEAERSQRRRLLVPANEGDPTETLRQCFDDFADLVPFDSW | |
| | | EPLMRKLGLMDNEIKVAKAEAAGHRDTLYTMLIKWVNKTGRDA | |
| | | SVHTLLDALETLGERLAKQKIEDHLLSSGKFMYLEGNADSAMS | |
| SEQ ID | Cynomolgus | MGQLRQSAPAASGARKGRGPGPREARGARPGLRVLKTLVLVVA | |
| NO: 39 | monkey DR5 | AARVLLSVSADCAPITRQSLDPQRRAAPQQKRSSPTEGLCPPGH | |
| | (NCBI | HISEDSRECISCKYGQDYSTHWNDFLFCLRCTKCDSGEVEVNSCT | |
| | XP_005562887.1) | TTRNTVCQCEEGTFREEDSPEICRKCRTGCPRGMVKVKDCTPWS | |
| | | DIECVHKESGTKHTGEVPAVEKTVTTSPGTPASPCSLSGIIIGVIVL | |
| | | VVIVVVAVIVWKTSLWKKVLPYLKGVCSGGGGDPERVDSSSHSP | |
| | | QRPGAEDNALNEIVSIVQPSQVPEQEMEVQEPAEQTDVNTLSP | |
| | | GESEHLLEPAKAEGPQRRGQLVPVNENDPTETLRQCFDDFAAIV | |
| | | PFDAWEPLVRQLGLTNNEIKVAKAEAASSRDTLYVMLIKWVNKT | |
| | | GRAASVNTLLDALETLEERLAKQKIQDRLLSSGKFMYLEDNADSA | |
| | | TS | |
| SEQ ID | Cyno DR5Mfdel- | MGQLRQSAPAASGARKGRGPGPREARGARPGLRVLKTLVLVVA | Cynomolgus |
| NO: 40 | K420N | AARVLLSVSADCAPITRQSLDPQRRAAPQQKRSSPTEGLCPPGH | monkey DRS |
| | | HISEDSRECISCKYGQDYSTHWNDFLFCLRCTKCDSGEVEVNSCT | with deletion |
| | | TTRNTVCQCEEGTFREEDSPEICRKCRTGCPRGMVKVKDCTPWS | of aa 185-213 |
| | | DIECVHKESGIIIGVIVLVVIVVVAVIVWKTSLWKKVLPYLKGVCSG | and K420N |
| | | GGGDPERVDSSSHSPQRPGAEDNALNEIVSIVQPSQVPEQEME | mutation as |
| | | VQEPAEQTDVNTLSPGESEHLLEPAKAEGPQRRGQLVPVN | END used in the |
| | | PTETLRQCFDDFAAIVPFDAWEPLVRQLGLTNNEIKVAKAEAASS | examples |
| | | RDTLYVMLIKWVNKTGRAASVNTLLDALETLEERLANQKIQDRLL | |
| | | SSGKFMYLEDNADSATS | |
| SEQ ID | Murine DRS | MEPPGPSTPTASAAARADHYTPGLRPLPKRRLLYSFALLLAVLQA | |
| NO: 41 | (Uniprot | VFVPVTANPAHNRPAGLQRPEESPSRGPCLAGQYLSEGNCKPCR | |
| | Q9QZM4) | EGIDYTSHSNHSLDSCILCTVCKEDKVVETRCNITTNTVCRCKPGT | |
| | | FEDKDSPEICQSCSNCTDGEEELTSCTPRENRKCVSKTAWASWH | |
| | | KLGLWIGLLVPVVLLIGALLVWKTGAWRQWLLCIKRGCERDPES | |
| | | ANSVHSSLLDRQTSSTTNDSNHNTEPGKTQKTGKKLLVPVNGN | |
| | | DSADDLKFIFEYCSDIVPFDSWNRLMRQLGLTDNQIQMVKAETL | |
| | | VTREALYQMLLKWRHQTGRSASINHLLDALEAVEERDAMEKIED | |
| | | YAVKSGRFTYQNAAAQPETGPGGSQCV | |
| SEQ ID | DR5ECD- | MEQRGQNAPAASGARKRHGPGPREARGARPGLRVPKTLVLVV | Extracellular |
| NO: 42 | FcRbHisCtag | AAVLLLVSAESALITQQDLAPQQRVAPQQKRSSPSEGLCPPGHHI | domain of |
| | | SEDGRDCISCKYGQDYSTHWNDLLFCLRCTRCDSGEVELSPCTTT | human DRS |
| | | RNTVCQCEEGTFREEDSPEMCRKCRTGCPRGMVKVGDCTPWS | natural variant |
| | | DIECVHKESGTKHSGEAPAVEETVTSSPGTPASPCSAPSTCSKPTC | P32L; A67V; |
| | | PPPELLGGPSVFIFPPKPKDTLMISRTPEVTCVVVDVSQDDPEVQ | V191A fused |
| | | FTWYINNEQVRTARPPLREQQFNSTIRVVSTLPIAHQDWLRGKE | to rabbit Fc |
| | | FKCKVHNKALPAPIEKTISKARGQPLEPKVYTMGPPREELSSRSVS | domain, His |
| | | LTCMINGFYPSDISVEWEKNGKAEDNYKTTPAVLDSDGSYFLYSK | tag and C-tag |
| | | LSVPTSEWQRGDVFTCSVMHEALHNHYTQKSISRSPGKHHHHH | (at C- |
| | | HHHEPEA | terminus) |
| SEQ ID | DR5sh79- | MEQRGQNAPAASGARKRHGPGPREARGARPGPRVPKTLVLVV | |
| NO: 43 | 115ECDdel- | AAVLLLVSAESALITQQDLAPQQRAAPQQKRSSPSEGPCLAGQY | |
| | FcRbHisCtag | LSEGNCKPCREGIDYTSHSNHSLDSCILCTRCDSGEVELSPCTTTR | |
| | | NTVCQCEEGTFREEDSPEMCRKCRTGCPRGMVKVGDCTPWSD | |
| | | IECVHKESGAPSTCSKPTCPPPELLGGPSVFIFPPKPKDTLMISRTP | |
| | | EVTCVVVDVSQDDPEVQFTWYINNEQVRTARPPLREQQFNSTI | |
| | | RVVSTLPIAHQDWLRGKEFKCKVHNKALPAPIEKTISKARGQPLE | |
| | | PKVYTMGPPREELSSRSVSLTCMINGFYPSDISVEWEKNGKAED | |
| | | NYKTTPAVLDSDGSYFLYSKLSVPTSEWQRGDVFTCSVMHEALH | |
| | | NHYTQKSISRSPGKHHHHHHHHEPEA | |
| SEQ ID | DR5sh139- | MEQRGQNAPAASGARKRHGPGPREARGARPGPRVPKTLVLVV | |
| NO: 44 | 166ECDdel- | AAVLLLVSAESALITQQDLAPQQRAAPQQKRSSPSEGLCPPGHHI | |
| | FcRbHisCtag | SEDGRDCISCKYGQDYSTHWNDLLFCLRCTRCDSGEVELSPCTTT | |
| | | RNTVCQCKPGTFEDKDSPEICQSCSNCTDGEEEVGDCTPWSDIE | |
| | | CVHKESGAPSTCSKPTCPPPELLGGPSVFIFPPKPKDTLMISRTPE | |
| | | VTCVVVDVSQDDPEVQFTWYINNEQVRTARPPLREQQFNSTIR | |
| | | VVSTLPIAHQDWLRGKEFKCKVHNKALPAPIEKTISKARGQPLEP | |
| | | KVYTMGPPREELSSRSVSLTCMINGFYPSDISVEWEKNGKAEDN | |
| | | YKTTPAVLDSDGSYFLYSKLSVPTSEWQRGDVFTCSVMHEALHN | |
| | | HYTQKSISRSPGKHHHHHHHHEPEA | |
| SEQ ID | kDR5ECDdelHis | MGWSCIILFLVATATGVHSQQDLAPQQRAAPQQKRSSPSEGLC | DR5ECDdelHis |
| NO: 45 | | PPGHHISEDGRDCISCKYGQDYSTHWNDLLFCLRCTRCDSGEVE | where the |
| | | LSPCTTTRNTVCQCEEGTFREEDSPEMCRKCRTGCPRGMVKVG | signal peptide |
| | | DCTPWSDIECVHKESGHHHHHHHH | (aa 1-57) is |
| | | | replaced by |
| | | | that of Kappa |
| | | | 2F8 to |
| | | | introduce |
| | | | defined |
| | | | domain |
| | | | boundaries |
| SEQ ID | Fc IgG1m(f) | ASTKGPSVFPLAPSSKSTSGGTAALGCLVKDYFPEPVTVSWNSGA | |
| NO: 46 | | LTSGVHTFPAVLQSSGLYSLSSVVTVPSSSLGTQTYICNVNHKPSN | |
| | | TKVDKRVEPKSCDKTHTCPPCPAPELLGGPSVFLFPPKPKDTLMI | |
| | | SRTPEVTCVVVDVSHEDPEVKFNWYVDGVEVHNAKTKPREEQY | |
| | | NSTYRVVSVLTVLHQDWLNGKEYKCKVSNKALPAPIEKTISKAKG | |
| | | QPREPQVYTLPPSREEMTKNQVSLTCLVKGFYPSDIAVEWESNG | |
| | | QPENNYKTTPPVLDSDGSFFLYSKLTVDKSRWQQGNVFSCSVM | |
| | | HEALHNHYTQKSLSLSPGK | |
| SEQ ID | HC hDR5-01- | EVQLQQSGAEVVKPGASVKLSCKASGFNIKDTFIHWVKQAPGQ | Clone IgG1- |
| NO: 47 | G56T-E430G | GLEWIGRIDPANTNTKYDPKFQGKATITTDTSSNTAYMELSSLRS | hDR5-01- |
| | | EDTAVYYCVRGLYTYYFDYWGQGTLVTVSSASTKGPSVFPLAPSS | G56T-E430G |
| | | KSTSGGTAALGCLVKDYFPEPVTVSWNSGALTSGVHTFPAVLQS | Without lysine |
| | | SGLYSLSSVVTVPSSSLGTQTYICNVNHKPSNTKVDKRVEPKSCD | (K) |
| | | KTHTCPPCPAPELLGGPSVFLFPPKPKDTLMISRTPEVTCVVVDV | |
| | | SHEDPEVKFNWYVDGVEVHNAKTKPREEQYNSTYRVVSVLTVL | |
| | | HQDWLNGKEYKCKVSNKALPAPIEKTISKAKGQPREPQVYTLPP | |
| | | SREEMTKNQVSLTCLVKGFYPSDIAVEWESNGQPENNYKTTPPV | |
| | | LDSDGSFFLYSKLTVDKSRWQQGNVFSCSVMHGALHNHYTQKS | |
| | | LSLSPG | |
| SEQ ID | HC hDR5-05- | QVQLVQSGAEVKKPGASVKVSCKASGFNIKDTHMHWVRQAPG | Clone IgG1- |
| NO: 48 | E430G | QRLEWIGRIDPANGNTEYDQKFQGRVTITVDTSASTAYMELSSL | hDR5-05-E430G |
| | | RSEDTAVYYCARWGTNVYFAYWGQGTLVTVSSASTKGPSVFPL | Without lysine |
| | | APSSKSTSGGTAALGCLVKDYFPEPVTVSWNSGALTSGVHTFPA | (K) |
| | | VLQSSGLYSLSSVVTVPSSSLGTQTYICNVNHKPSNTKVDKRVEP | |
| | | KSCDKTHTCPPCPAPELLGGPSVFLFPPKPKDTLMISRTPEVTCVV | |
| | | VDVSHEDPEVKFNWYVDGVEVHNAKTKPREEQYNSTYRVVSVL | |
| | | TVLHQDWLNGKEYKCKVSNKALPAPIEKTISKAKGQPREPQVYTL | |
| | | PPSREEMTKNQVSLTCLVKGFYPSDIAVEWESNGQPENNYKTTP | |
| | | PVLDSDGSFFLYSKLTVDKSRWQQGNVFSCSVMHGALHNHYTQ | |
| | | KSLSLSPG | |

The present invention is further illustrated by the following Examples which should not be construed as further limiting.

### EXAMPLES

### Example 1: Antibody and Antigen Constructs

### Expression Constructs for DR5

Codon-optimized constructs for expression of the short isoform of human DR5 (SEQ ID NO 36; based on UniprotKB/Swiss-Prot 014763-2) with death domain loss-of-function mutation K386N, and cynomolgus monkey DR5 (SEQ ID NO 40; based on NCBI accession number XP 005562887.1) with deletion of amino acids 185-213 and death domain loss-of-function mutation K420N, were generated. The constructs were cloned in the mammalian expression vector pcDNA3.3 (Invitrogen). For mapping of the binding regions of the DR5 antibodies (as described in Example 4) the following chimeric human/mouse DR5 constructs were made (human DR5 in which, respectively, the following parts were replaced by the corresponding mouse DR5 sequence with numbers referring to the human sequence): construct A aa 56-68, construct B aa 56-78, construct C aa 69-78, construct D aa 79-115, construct E 79-138, construct F aa 97-138, construct G aa 139-166, construct H aa 139-182, construct I aa 167-182, construct J 167-210, construct K aa 183-210. DR5 expression constructs were transiently transfected in Freestyle CHO-S cells (Life technologies, Cat no R80007), using the FreeStyle MAX Reagent (Invitrogen by Life technologies, Cat no 16447-100), as described by the manufacturer. Transfected cells were stored in liquid nitrogen.

Codon-optimized construct for the extracellular domain (ECD) of human DR5 with a C-terminal tag were generated: DR5ECD-FcRbHisCtag (SEQ ID NO 42) and kDR5ECDdelHis (SEQ ID NO 45). All constructs contained suitable restriction sites for cloning and an optimal Kozak (GCCGCCACC) sequence. The constructs were cloned in the mammalian expression vector pcDNA3.3 (Invitrogen).

### Expression Constructs for Antibodies

For antibody expression the VH and VL sequences of the chimeric human/mouse DR5 antibodies DR5-01 and DR5-05 (EP2684896A1; WO17093448; US20170260281) and their humanized variants hDR5-01 and hDR5-05 (WO14009358; WO17093448; US20170260281) were cloned in expression vectors (pcDNA3.3) containing the relevant constant HC and LC regions. Desired mutations were introduced either by gene synthesis or site directed mutagenesis.

In some of the examples gp120-specific human IgG1 antibody IgG1-b12 or IgG1-b12-E430G was used as negative (isotype) control (Barbas et al., J Mol Biol. 1993 Apr. 5; 230(3):812-23).

### Transient Expression

Antibodies were expressed as IgG1,κ by GeneArt or in house by Genmab BV. At Genmab, plasmid DNA mixtures encoding both heavy and light chains of antibodies were transiently transfected in Expi293F cells (Life technologies) using 293fectin (Life technologies), essentially as described by Vink et al. (Vink et al., Methods, 65 (1), 5-10 2014).

Membrane proteins were expressed in Freestyle CHO-S cells (Life technologies), using the freestyle Max reagent, as described by the manufacturer.

### Purification and Analysis of Proteins

Antibodies were purified by immobilized protein A chromatography. His-tagged recombinant protein was purified by immobilized metal affinity chromatography. Protein batches were analyzed by a number of bioanalytical assays including sodium dodecyl sulphate polyacrylamide gel electrophoresis (SDS-PAGE), size exclusion chromatography (SEC) and measurement of endotoxin levels.

### Example 2: DR5 Antibody Binding to Transfected CHO-S Cells

Frozen transfected CHO-S cells were quickly thawed at 37° C. and suspended in 10 mL medium (RPMI 1640 with 25 mM Hepes and L-Glutamine [Lonza, Cat no BE12-115F]+50 Units penicillin/50 Units streptomicin [Pen/Strep; Lonza, Cat no DE17-603E]+10% heat-inactivated Donor Bovine Serum with Iron [DBSI; Life Technologies, Cat no 10371-029]). Cells were washed with PBS and resuspended in FACS buffer (PBS+0.1% w/v bovine serum albumin [BSA; Roche, Cat no 10735086001]+0.02% w/v sodium azide) at a concentration of 1.0×10⁶ cells/mL. 100 μL cell suspension samples (100,000 or 50,000 cells per well) were seeded in 96-well ps plates (Greiner Bio-One, Cat no 650101) and pelleted by centrifugation at 300×g for 3 minutes at 4° C. 25 μL of dilution antibody preparation series (0-20 μg/mL final antibody concentrations in 6-fold dilutions) was added and incubated for 30 minutes at 4° C. Next, cells were washed once with 150 μL FACS buffer and incubated with 50 μL secondary antibody R-phycoerythrin (R-PE)-conjugated goat-anti-human IgG F(ab′)₂ (Jackson ImmunoResearch, Cat no 109-116-098; 1/100) for 30 minutes at 4° C. protected from light. Cells were washed once with 150 μL FACS buffer, resuspended in 50 μL or 100 μL FACS buffer, and antibody binding was analysed by flow cytometry on a BD LRSFFortessa cell analyzer (BD Biosciences) by recording 10,000 events. Transfection efficacy for the CHO-S cells was not 100%; therefore the geometric mean fluorescence intensity (FI) of the PE positive population was determined. In case the PE positive population could no longer be discriminated from the negative population geometric mean FI from all cells was determined. Binding curves were analyzed using non-linear regression analysis (sigmoidal dose-response with variable slope) using GraphPad Prism software.

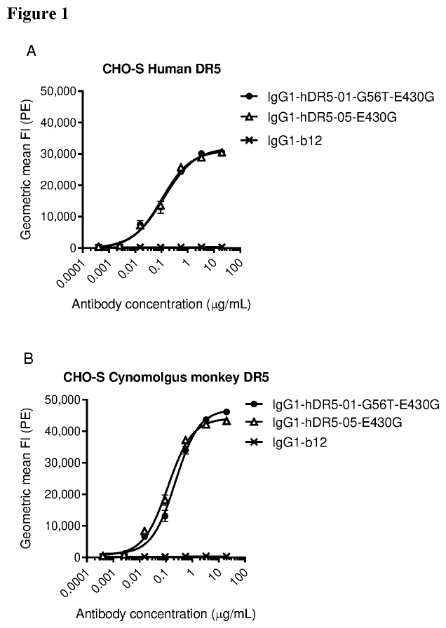

IgG1-hDR5-01-G56T-E430G and IgG1-hDR5-05-E430G showed similar dose-dependent binding to CHO-S cells expressing human and cynomolgus monkey DR5, with apparent affinities (EC50) in the high picomolar-low nanomolar range (FIG. 1, Table 3).

TABLE 3
EC50 values for antibody binding to human and cynomolgus
monkey DR5. Antibody binding was tested by flow cytometry
using CHO-S cells expressing human DR5 and cynomolgus
monkey DR5. EC50 values were calculated from the dose
response-curve using GraphPad Prism software. Average EC50
values were calculated from four independent experiments.

| | | | EC50 for cynomolgus | |
|---|---|---|---|---|
| | EC50 for human DR5 | | monkey DR5 | |
| | Average (n = 4) | | Average (n = 4) | |
| | μg/mL | nM | μg/mL | nM |
| Antibody | (SD) | (SD) | (SD) | (SD) |
| IgG1-hDR5-01-G56T- | 0.13 (0.03) | 0.87 (0.23) | 0.27 (0.18) | 1.77 (1.16) |
| E430G | | | | |
| IgG1-hDR5-05-E430G | 0.12 (0.03) | 0.83 (0.18) | 0.17 (0.08) | 1.16 (0.56) |

### Example 3: DR5 Antibody Binding to HCT 116

To prepare single cell suspensions, adherent HCT 116 cells (ATCC CCL-247) were washed twice with PBS (B.Braun, Cat no 3623140) before incubating with Trypsin-EDTA (Gibco, Cat no 15400-054, diluted in PBS to a final concentration of 0.05% Trypsin) for 2 minutes at 37° C. 10 mL culture medium (McCoy's 5A medium with L-Glutamine and HEPES [Lonza, Cat no BE12-168F]+10% Donor Bovine Serum with Iron [Life Technologies, Cat no 10371-029]+50 Units Penicillin/50 Units Streptomycin [Lonza, Cat no DE17-603E]) was added before pelleting the cells by centrifugation for 5 minutes at 1,200 rpm. Cells were resuspended in 10 mL medium, pelleted again by centrifugation for 5 minutes at 1,200 rpm, and resuspended in FACS buffer at a concentration of 1.0×10⁶ cells/mL. The next steps were performed at 4° C. 100 μL cell suspension samples (100,000 cells per well) were seeded in polystyrene 96-well round-bottom plates (Greiner Bio-One, Cat no 650101) and pelleted by centrifugation at 300×g for 3 minutes at 4° C. Cells were resuspended in 100 μL antibody samples of a dilution series (0-10 μg/mL in 5-fold dilutions) and incubated for 30 minutes at 4° C. Cells were pelleted by centrifugation at 300×g for 3 minutes at 4° C. and washed twice with 150 μL FACS buffer. Cells were incubated with 50 μL secondary antibody R-phycoerythrin (R-PE)-conjugated goat-anti-human IgG F(ab′)2 (Jackson ImmunoResearch, Cat no 109-116-098; 1/100) for 30 minutes at 4° C., protected from light. Cells were washed twice with 150 μL FACS buffer, resuspended in 150 μL FACS buffer, and antibody binding was analyzed by flow cytometry on a FACS Fortessa (BD Biosciences) by recording 10,000 events. Binding curves were analyzed using non-linear regression analysis (sigmoidal dose-response with variable slope) using GraphPad Prism software.

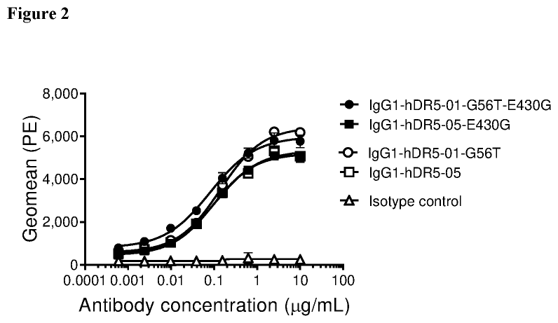

Binding to DR5-positive HCT 116 cells was observed for all tested DR5-specific antibodies, in a concentration-dependent manner (FIG. 2), with apparent affinity (EC50) values in the high picomolar/low nanomolar range (Table 4). EC50 values were in the same range for IgG1-hDR5-01-G56T-E430G and IgG1-hDR5-05-E430G. The E430G mutation did not affect binding to DR5 as demonstrated by highly comparable EC50 values for the HexaBody molecules and their respective WT counterparts. Control antibody IgG1-b12 showed no binding.

TABLE 4
EC50 values for antibody binding to HCT116. Antibody
binding was tested by flow cytometry using HCT 116
human colorectal carcinoma cells. EC50 values were
calculated from the dose response-curve using GraphPad
Prism software.

| | EC50 HCT 116 | | |
|---|---|---|---|
| | Average | | |
| Antibody | μg/mL (SD) | nM (SD) | n |
| IgG1-hDR5-01-G56T | 0.12 (0.04) | 0.82 (0.28) | 5 |
| IgG1-hDR5-01-G56T-E430G | 0.08 (0.06) | 0.51 (0.39) | 6 |
| IgG1-hDR5-05 | 0.11 (0.04) | 0.76 (0.29) | 5 |
| IgG1-hDR5-05-E430G | 0.10 (0.05) | 0.70 (0.35) | 6 |

### Example 4: Mapping of Binding Regions Using Domain-Swapped DR5 Molecules

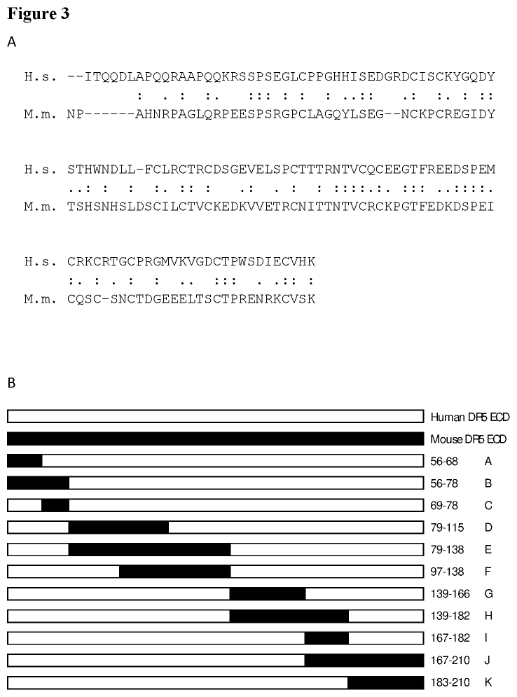

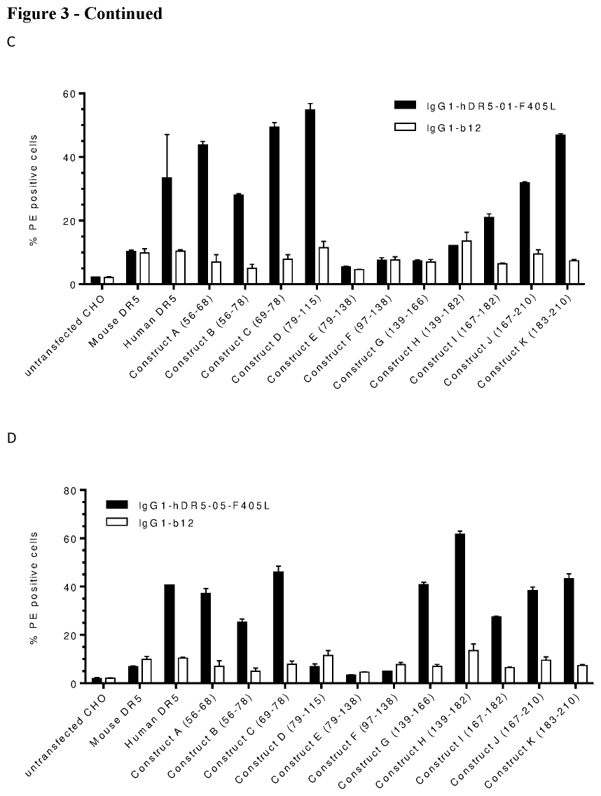

The amino acid sequences of the extracellular domains of human and murine DR5 show limited homology (FIG. 3A) and the humanized antibodies IgG1-hDR5-01-F405L and IgG1-hDR5-05-F405L do not bind murine DR5 (FIG. 3C,D). With the aim to identify amino acid stretches in the human DR5 extracellular domain that are involved in antibody binding, we developed eleven human-mouse chimeric DR5 molecules, in which specific human DR5 domains had been replaced by the mouse analogues (domain-swapped DR5 molecules described in Example 1) as visualized in FIG. 3B. The domain-swapped DR5 variants were transiently expressed on CHO cells. Loss of binding of the DR5 antibodies to domain-swapped DR5 molecules indicates that the swapped domain of human DR5 contains one or more amino acids that are crucial for binding. Vice versa, retention of binding of the DR5 antibodies to domain-swapped DR5 molecules indicates that the swapped domain of human DR5 does not contain amino acids that are crucial for binding. For the binding assay, 3×10⁶ transfected cells were washed and resuspended in 3 mL FACS buffer. 100 μL cell suspension was added per well (100,000 cells per well) of 96-well round bottom plates (Greiner Bio-one; Cat no 650101). The next steps were performed at 4° C. Cells were pelleted, resuspended in 50 μL DR5 antibody sample (10 μg/mL final concentration) and incubated for 30 minutes at 4° C. The cells were washed twice and incubated in 50 μL secondary antibody R-PE-conjugated goat-anti-human IgG F(ab′)₂ (Jackson ImmunoResearch; Cat no 109-116-098; 1/100) for 30 minutes at 4° C. protected from light. Cells were washed twice, resuspended in 120 μL FACS buffer, and analyzed by flow cytometry on a FACS Canto II (BD Biosciences). The percentage of viable PE-positive cells was plotted using GraphPad Prism software. Surface expression was confirmed for each domain-swapped DR5 molecule using a panel of DR5 antibodies directed against different epitopes (not shown). The non-target binding antibody IgG1-b12 against gp120 was included as a negative control for binding. FIG. 3C shows that IgG1-hDR5-01-F405L showed loss of binding to constructs E (79-138), F (97-138), G (139-166) and H (139-182), whereas binding to constructs A-D (covering human DR5 sequence 56-115) and I-K (covering human DR5 sequence 167-210) was retained. Together, these data indicate that the amino acid regions 116-138 and 139-166 each contain one or more amino acids required for binding of IgG1-hDR5-01-F405L to human DR5. FIG. 3D shows that IgG1-hDR5-05-F405L showed loss of binding to constructs D (79-115), E (79-138) and F (97-138), whereas binding to constructs A-C (covering human DR5 sequence 56-78) and G-K (covering human DR5 sequence 139-210) was retained. Together, these data indicate that the amino acid region 79-138 contains one or more amino acids required for binding of IgG1-hDR5-05-F405L to human DR5.

### Example 5: ELISA for Selective Antibody Detection

Based on the specific DR5 binding regions of IgG1-hDR5-01-F405L and IgG1-hDR5-05-F405L that were identified by binding analysis to human-mouse DR5 shuffle variants (Example 4), two recombinant DR5 antigens were produced based on DR5ECD-FcRbHisCtag: one with the human DR5 amino acids 79-115 replaced by the corresponding mouse sequence (DR5sh79-115ECDdel-FcRbHisCtag) and one with the human DR5 amino acids 133-166 replaced by the corresponding mouse sequence (DR5sh139-166ECDdel-FcRbHisCtag).

96-well flat-bottom ELISA plates (Greiner bio-one, Cat no 665092) were coated overnight (4° C.) with 100 μL (2 μg/mL in PBS) penta-his antibody (Qiagen, Cat no 34660). After washing the plates three times in PBST, non-specific binding was blocked with 200 μL/well PBS with 1% BSA and incubated for 1 hour while shaking (300×rpm) at RT. After washing three times in PBST, recombinant DR5 ECD fusion proteins (DR5ECD-FcRbHisCtag; DR5sh79-115ECDdel-FcRbHisCtag; DR5sh139-166ECDdel-FcRbHisCtag) were added to the wells (1 μg/mL, 100 μL/well) and incubated for 1 hour while shaking (300×rpm) at RT. After washing plates three times in PBST, three-fold serial dilutions of DR5-specific antibodies or isotype control (range 0.46-1,000 ng/mL) in PBS containing 2% BSA were added in duplicate (100 μL) and incubated for 2 hours while shaking (300×rpm) at RT. After washing three times in PBST, wells were incubated with 100 μL HRP-conjugated goat anti-human IgG F(ab)₂ (Jackson, Cat no 109-035-097), and incubated for 1 hour while shaking (300×rpm) at RT. After washing three times with PBST, the reaction was visualized through incubation with 100 μL ABTS (Roche, Cat no 11112597001) for 30 minutes at RT protected from light. The substrate reaction was stopped by adding an equal volume of 2% (w/v) oxalic acid. Fluorescence was measured at 405 nm on an ELISA reader (BioTek ELx808 Absorbance Microplate Reader).

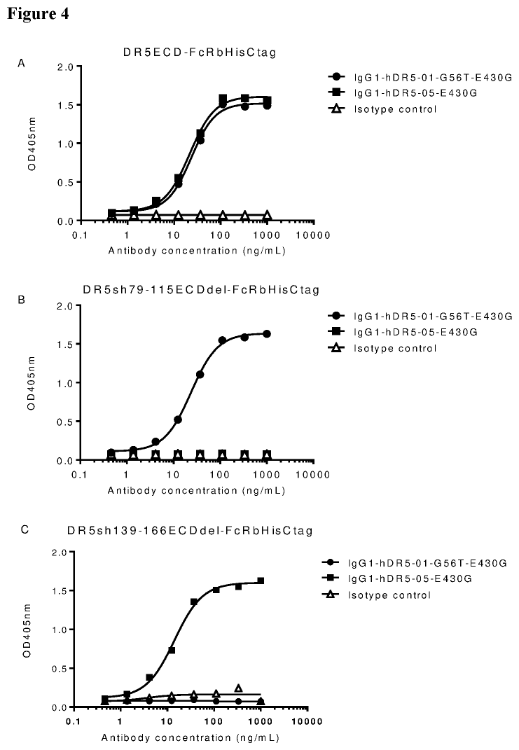

Concentration-dependent binding was observed for IgG1-hDR5-01-G56T-E430G and IgG1-hDR5-05-E430G to the fully human DR5 antigen (DR5ECD-FcRbHisCtag, FIG. 4A). When plates were coated with DR5sh79-115ECDdel-FcRbHisCtag, concentration-dependent binding was only observed with IgG1-hDR5-01-G56T-E430G, but not with IgG1-hDR5-05-E430G (FIG. 4B). Vice versa, when plates were coated with DR5sh139-166ECDdel-FcRbHisCtag, concentration-dependent binding was only observed with IgG1-hDR5-05-E430G, but not with IgG1-hDR5-01-G56T-E430G (FIG. 4C). These experiments show that with these binding assays, the presence of IgG1-hDR5-01-G56T-E430G can be specifically demonstrated using DR5sh79-115ECDdel-FcRbHisCtag protein, and the presence of IgG1-hDR5-05-E430G can be specifically demonstrated using DR5sh139-166ECDdel-FcRbHisCtag protein.

### Example 6: Affinity Determination Using Bio-Layer Interferometry

Target binding affinities of DR5-specific antibodies were determined using Bio-Layer Interferometry on a ForteBio Octet HTX instrument. To this end, antibodies (1 μg/mL) were loaded onto Anti-Human IgG Fc Capture (AHC) biosensors (ForteBio) for 600 s. After a baseline (100 s) in Sample Diluent (ForteBio), the association (1,000 s) and dissociation (1,000 s) of kDR5ECDdelHis was determined, using a concentration range of 100 nM-1.56 nM (1.53 μg/mL-0.02 μg/mL) with 2-fold dilution steps. Experiments were carried out while shaking (1,000 rpm) at 30° C. Affinity data was analyzed with ForteBio Data Analysis Software, using the 1:1 model and a global full fit with 1,000 s association time and 100 s dissociation time as window of interest. Data traces were corrected by subtraction of a reference curve (Sample Diluent instead of kDR5ECDdelHis), the Y-axis was aligned to the last 10 s of the baseline and interstep correction as well as Savitzky-Golay filtering was applied. Data traces with a response <0.05 nm were excluded from analysis.

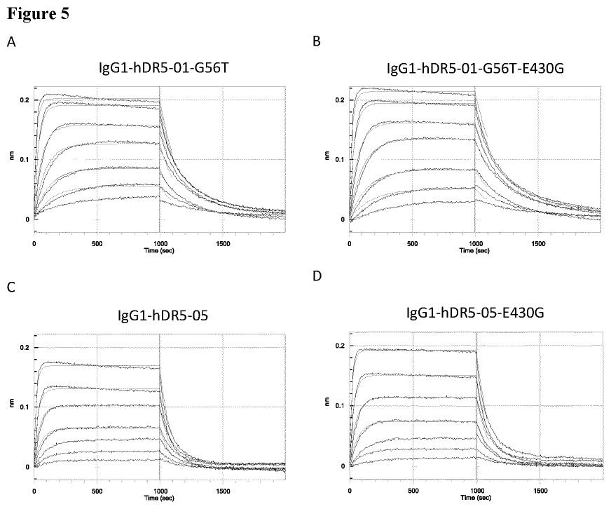

DR5 binding affinity of IgG1-hDR5-01-G56T-E430G and IgG1-hDR5-05-E430G was determined and compared to the corresponding WT antibodies (FIG. 5). Data show that the dissociation constants (K_{D}) of the DR5-specific antibodies were all in the nanomolar range, and independent of the presence of the hexamerization-enhancing mutation E430G (Table 5). The affinity of IgG1-hDR5-01-G56T-E430G (K_{D} 10.6 nM) was approximately 2.5-fold higher than for IgG1-hDR5-05-E430G (K_{D} 27.1 nM).

TABLE 5
Average values of antibody binding affinity for human recombinant DR5 as tested by
Bio-Layer interferometry.

| | Bio-Layer Interferometry | | | |
|---|---|---|---|---|
| | K_{D}¹ | kₐ² | k_{d}³ | |
| Antibody | nM (SD) | M⁻¹ × sec⁻¹ (SD) | sec⁻¹ (SD) | n |
| IgG1-hDR5-01-G56T | 12.8 (2.7) | 4.3 × 10⁵ (2.8 × 10⁴) | 5.5 × 10⁻³ (8.0 × 10⁻⁴) | 2 |
| IgG1-hDR5-01-G56T-E430G | 10.6 (0.6) | 4.2 × 10⁵ (2.3 × 10⁴) | 4.5 × 10⁻³ (5.4 × 10⁻⁵) | 3 |
| IgG1-hDR5-05 | 28.5 (0.3) | 4.4 × 10⁵ (2.8 × 10⁴) | 1.3 × 10⁻² (9.9 × 10⁻⁴) | 2 |
| IgG1-hDR5-05-E430G | 27.1 (3.3) | 4.2 × 10⁵ (3.7 × 10⁴) | 1.1 × 10⁻² (2.0 × 10⁻³) | 3 |

¹K_{D} Dissociation constant
²kₐ association rate constant or on-rate
³k_{d} dissociation rate constant or off-rate

### Example 7: Viability Assays Human Cell Lines

The capacity of the mixture of IgG1-hDR5-01-G56T-E430G and IgG1-hDR5-05-E430G to induce cytotoxicity was explored in viability assays in vitro using 12 different cell lines. COLO 205 cancer cells were harvested by pooling the culture supernatant containing non-adherent cells and trypsinized adherent cells. A375, A549, BxPC-3, HPAFII, PANC1, HCT 116, HCT 15, HT29, SW480, SK-MES-1 and SNUS cancer cells were harvested by trypsinization. For trypsinization, adherent cells were incubated with Trypsin-EDTA (Gibco, Cat no 15400-054) diluted in PBS (B.Braun; Cat no 3623140) to a final concentration of 0.05% Trypsin for 2 minutes at 37° C. and passed through a cell strainer. Cells were pelleted by centrifugation for 5 minutes at 1,200 rpm and resuspended at a concentration of 0.5×10⁵ cells/mL in culture medium (see Table 6).

TABLE 6
Cell lines used in this example

| | | Company, | |
|---|---|---|---|
| Cell line | Origin | Cat no | Culture medium |
| A375 | Melanoma | ATCC | DMEM 4.5 g/L Glucose without L-Glutamine with HEPES |
| | | CRL-1619 | (Lonza, Cat no BE12-709F) + 2 mM L-glutamine |
| | | | (Lonza, Cat no BE17-605F) + 10% heat-inactivated |
| | | | Donor Bovine Serum with Iron (DBSI; Life Technologies |
| | | | Cat no 10371-029) + 50 Units/mL penicillin/50 Units/mL |
| | | | streptomicin (Pen/Strep; Lonza Cat no DE17-603E) |
| Bx-PC-3 | Pancreatic | ATCC | RPMI 1640 with 25 mM Hepes and L-Glutamine (Lonza, |
| | cancer | CRL-1687 | Cat no BE12-115F) + 10% heat-inactivated DBSI + Pen/Strep |
| HPAF-II | Pancreatic | ATCC | EMEM with low sodium bicarbonate (ATCC, Cat no 30- |
| | cancer | CRL-1997 | 2003) + 10% heat-inactivated DBSI + Pen/Strep |
| PANC-I | Pancreatic | ATCC | DMEM 4.5 g/L Glucose without L-Glutamine and HEPES |
| | cancer | CRL-1469 | (Lonza) + 2 mM L-glutamine (Lonza) + 1 mM Sodium |
| | | | Pyruvate (Lonza, Cat no BE13-115E) + 10% heat-inactivated |
| | | | DBSI + Pen/Strep |
| COLO 205 | Colorectal | ATCC | RPMI 1640 with 25 mM Hepes and L-Glutamine (Lonza) + |
| | cancer | CCL-222 | 10% heat-inactivated DBSI + Pen/Strep |
| HCT 116 | Colorectal | ATCC | McCoy's 5A medium with L-Glutamine and HEPES |
| | cancer | CCL-247 | (Lonza, Cat no BE12-168F) + 10% heat-inactivated |
| | | | DBSI + Pen/Strep |
| HCT-15 | Colorectal | ATCC | RPMI 1640 with 25 mM Hepes and L-Glutamine (Lonza) + |
| | cancer | CCL-225 | 10% heat-inactivated DBSI + Pen/Strep |
| HT-29 | Colorectal | ATCC | McCoy's 5A medium with L-Glutamine and HEPES |
| | cancer | HTB-38 | (Lonza) + 10% heat-inactivated DBSI + Pen/Strep |
| SW480 | Colorectal | ATCC | RPMI 1640 with 25 mM Hepes and L-Glutamine (Lonza) + |
| | cancer | CCL-228 | 10% heat-inactivated DBSI + Pen/Strep |
| A549 | Lung cancer- | ATCC | Ham's F-12K Medium with low sodium bicarbonate |
| | NSCLC | CCL-185 | (ATCC, Cat no 30-2004) + 2 mM L-glutamine |
| | | | (Lonza) + 10% heat-inactivated DBSI + Pen/Strep |
| SK-MES-1 | Lung cancer- | ATCC | EMEM with low sodium bicarbonate (ATCC) + 10% |
| | NSCLC | HTB-58 | heat-inactivated DBSI + Pen/Strep |
| SNU-5 | Gastric | ATCC | IMEM with HEPES and L-glutamine (Lonza, Cat no |
| | cancer | CRL-5973 | BE12-722F) + 10% heat-inactivated DBSI + Pen/Strep |

100 μL of single cell suspensions (5,000 cells per well) were seeded in polystyrene 96-well flat-bottom plates (Greiner Bio-One, Cat no 655180) and allowed to adhere overnight at 37° C. The following day, 50 μL antibody samples (0.002-133 nM final concentrations in 4-fold dilutions) were added to the adherent cells and incubated for 3 days at 37° C. As a positive control in all viability assays, cells were incubated with 5 μM staurosporine (Sigma Aldrich, Cat no S6942), and untreated cells were included as the negative control. The viability of the cultured cells was determined in a CellTiter-Glo Luminescent Cell Viability Assay (Promega, Cat no G7571) that quantifies the presence of ATP, which is an indicator of metabolically active cells. From the kit, 15 μL Luciferin Solution Reagent was added to each well of the viability assay plate. Next, plates were incubated for 1.5 hours at 37° C. 100 μL supernatant was transferred to a white OptiPlate-96 (Perkin Elmer, Cat no 6005299) and luminescence was measured on an EnVision Multilabel Reader (PerkinElmer). Data were analyzed and plotted using non-linear regression (sigmoidal dose-response with variable slope) using GraphPad Prism software. The percentage viable cells was calculated using the following formula: % viable cells=[(luminescence antibody sample-luminescence staurosporine sample)/(luminescence no antibody sample—luminescence staurosporine sample)]×100.

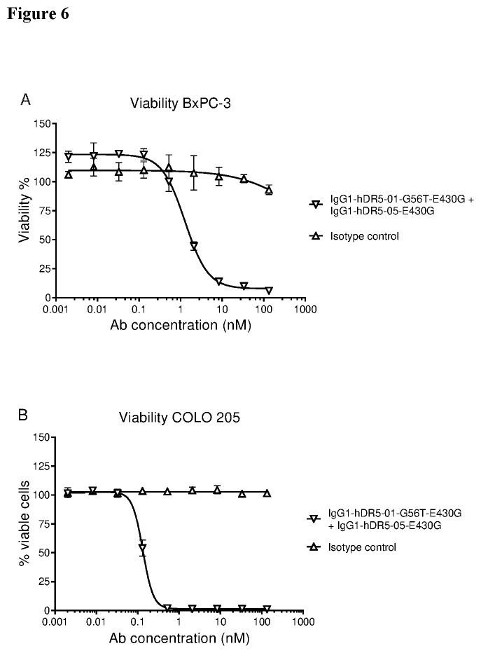

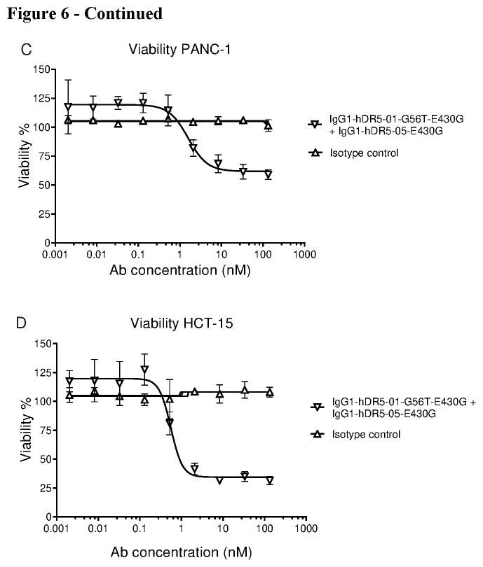

The mixture of IgG1-hDR5-01-G56T-E430G and IgG1-hDR5-05-E430G induced dose-dependent cytotoxicity, as shown in FIG. 6 for the colorectal cancer cell lines COLO 205 and HCT-15 and the pancreatic cancer cell lines BxPC-3 and PANC-1. An overview and calculation of the average values for IC20, IC50, IC90 and maximal inhibition of the tested cell lines (A375, A549, BxPC-3, HPAF-II, PANC-1, COLO 205, HCT 116, HCT-15, HT-29, SW480, SK-MES-1, SNU-5) is presented in Table 7.

TABLE 7
Average IC20, IC50 and IC90 values and percentage of maximal growth inhibition from
a three-day viability assay performed with 12 cell lines from different indications, to test
the cytotoxicity of the mixture of IgG1-hDR5-01-G56T-E430G and IgG1-hDR5-05-E430G.

| | | | | IC20 | IC50 | IC90 | Maximal |
|---|---|---|---|---|---|---|---|
| Cancer | | | | nM | nM | nM | inhibition |
| subtype | Cell line | n | | (μg/mL) | (μg/mL) | (μg/mL) | (%) |
| Melanoma | A375 | 2 | Average | 0.445 | 0.758 | 2.255 | 54.6 |
| | | | | (0.067) | (0.114) | (0.339) | |
| | | | SD | 0.343 | 0.266 | 0.976 | 31.9 |
| | | | | (0.052) | (0.040) | (0.147) | |
| Pancreatic | BxPC-3 | 10 | Average | 0.677 | 1.482 | 5.332 | 89.4 |
| | | | | (0.102) | (0.223) | (0.802) | |
| | | | SD | 0.313 | 0.576 | 1.945 | 3.6 |
| | | | | (0.047) | (0.087) | (0.293) | |
| | HPAF-II | 2* | | 8.219 | 14.620 | 36.420 | 19.5 |
| | | | | (1.236) | (2.198) | (5.477) | |
| | PANC-1 | 3 | Average | 0.554 | 1.534 | 8.578 | 61.0 |
| | | | | (0.083) | (0.231) | (1.290) | |
| | | | SD | 0.185 | 0.099 | 3.080 | 22.1 |
| | | | | (0.028) | (0.015) | (0.463) | |
| CRC | COLO 205 | 6 | Average | 0.103 | 0.163 | 0.339 | 99.6 |
| | | | | (0.015) | (0.024) | (0.052) | |
| | | | SD | 0.042 | 0.060 | 0.106 | 0.6 |
| | | | | (0.007) | (0.009) | (0.016) | |
| | HCT 116 | 4 | Average | 0.401 | 0.858 | 2.950 | 31.9 |
| | | | | (0.060) | (0.129) | (0.444) | |
| | | | SD | 0.347 | 0.668 | 1.819 | 21.2 |
| | | | | (0.052) | (0.101) | (0.274) | |
| | HCT-15 | 3 | Average | 0.692 | 1.396 | 4.336 | 66.7 |
| | | | | (0.104) | (0.210) | (0.652) | |
| | | | SD | 0.329 | 0.820 | 3.168 | 14.5 |
| | | | | (0.050) | (0.123) | (0.476) | |
| | HT-29 | 2 | Average | 2.152 | 5.173 | 21.240 | 15.9 |
| | | | | (0.324) | (0.778) | (3.194) | |
| | | | SD | 0.326 | 0.041 | 4.723 | 7.3 |
| | | | | (0.049) | (0.006) | (0.710) | |
| | SW480 | 4 | Average | 0.870 | 2.950 | 24.874 | 25.9 |
| | | | | (0.131) | (0.444) | (3.740) | |
| | | | SD | 0.520 | 1.202 | 18.587 | 5.8 |
| | | | | (0.078) | (0.181) | (2.795) | |
| NSCLC | A549 | 2 | | no efficacy | no efficacy | no efficacy | no efficacy |
| | SK-MES-1 | 2 | Average | 0.572 | 2.056 | 17.806 | 76.6 |
| | | | | (0.086) | (0.309) | (2.678) | |
| | | | SD | 0.055 | 0.562 | 13.780 | 11.4 |
| | | | | (0.008) | (0.085) | (2.072) | |
| Gastric | SNU-5 | 2 | Average | 0.274 | 1.261 | 21.465 | 44.7 |
| | | | | (0.041) | (0.190) | (3.228) | |
| | | | SD | 0.297 | 0.837 | 8.393 | 10.1 |
| | | | | (0.045) | (0.126) | (1.262) | |

*From the two experiments performed using HPAF-II cells (n = 2), no efficacy was observed in one experiment; 19.5% maximal inhibition was observed in the other experiment.

### Example 8: Viability Screening Using a Human Cancer Cell Line Panel: Solid Tumor indications

The cytotoxic capacity of the mixture of IgG1-hDR5-01-G56T-E430G and IgG1-hDR5-05-E430G was explored in a panel of 240 cell lines representing 16 solid cancer lineages: renal, lung mesothelioma, colorectal, gastric, pancreatic, liver, endometrial, ovarian, head and neck and urothelial cancer, triple negative breast cancer (TNBC), non-TNBC, NSCLC, small cell lung cancer (SCLC), melanoma and brain tumors (Table 8). The screening was performed in two sets at Horizon Discovery Ltd (screening 1 and screening 2). Frozen cells were thawed and expanded in growth media.

TABLE 8
Culturing conditions cell lines solid tumor indications at Horizon Discovery Ltd.

| Cell Line | TumorType | Culture Media |
|---|---|---|
| 5637 | Urothelial | RPMI with 10% FBS |
| HT-1197 | Urothelial | Eagles with 10% FBS |
| HT-1376 | Urothelial | Eagles MEM with 10% FBS |
| 382 | Urothelial | EMEM with 10% FBS |
| RT-112 | Urothelial | RPMI with 10% FBS |
| RT4 | Urothelial | McCoy's 5A with 10% FBS |
| SCaBER | Urothelial | EMEM with 10% FBS |
| SW780 | Urothelial | RPMI with 10% FBS |
| T-24 | Urothelial | McCoy's 5A with 10% FBS |
| TCCSUP | Urothelial | Eagles MEM with 10% FBS |
| UM-UC-3 | Urothelial | Eagles MEM with 10% FBS |
| A172 | Brain | Eagles MEM with 10% FBS |
| DBTRG-05MG | Brain | RMPI with 10% FBS with supplements |
| H4 | Brain | DMEM with 10% FBS |
| KNS-81 | Brain | DMEM:Ham's F12 (1:1) SERUM FREE |
| NMC-G1 | Brain | DMEM with 10% FBS |
| SF126 | Brain | Eagles MEM with 10% FBS |
| SF295 | Brain | RPMI with 10% FBS |
| SNB-75 | Brain | RPMI with 10% FBS |
| U-87 MG | Brain | Eagles MEM with 10% FBS |
| YH-13 | Brain | MEMa with 20% FBS |
| MCF7 | Breast, non-TNBC | Eagles MEM with 10% FBS and 0.01 mg/mL bovine |
| | | insulin |
| SK-BR-3 | Breast, non-TNBC | McCoy's 5A with 10% FBS |
| T47D | Breast, non-TNBC | RPMI with 10% FBS and 0.2 U/mL bovine insulin |
| BT-20 | Breast, TNBC | Eagles MEM with 10% FBS |
| BT-549 | Breast, TNBC | RPMI with 10% FBS and 0.023 IU/mL insulin |
| DU-4475 | Breast, TNBC | RPMI with 10% FBS |
| HCC1187 | Breast, TNBC | RPMI with 10% FBS |
| HCC1806 | Breast, TNBC | RPMI with 10% FBS |
| HCC1937 | Breast, TNBC | RPMI with 10% FBS |
| HCC38 | Breast, TNBC | RPMI with 10% FBS |
| HCC70 | Breast, TNBC | RPMI with 10% FBS |
| HMC-1-8 | Breast, TNBC | RPMI with 10% FBS |
| MDA-MB-231 | Breast, TNBC | RPMI with 10% FBS |
| MDA-MB-436 | Breast, TNBC | RPMI with 10% FBS and supplements |
| MDA-MB-453 | Breast, TNBC | RPMI with 10% FBS |
| MDA-MB-468 | Breast, TNBC | DMEM with 10% FBS |
| SUM159PT | Breast, TNBC | Ham's F12 with 5% FBS + supplements |
| C2BBe1 | Colorectal | DMEM with 10% FBS and 0.01 mg/mL human |
| | | transferrin |
| CL-11 | Colorectal | 1:1 DMEM, Ham's F12 with 20% FBS |
| CL-34 | Colorectal | 1:1 DMEM, Ham's F12 with 20% FBS |
| CL-40 | Colorectal | 1:1 DMEM, Ham's F12 with 20% FBS |
| COLO-201 | Colorectal | RPMI with 10% FBS |
| COLO-205 | Colorectal | RPMI with 10% FBS |
| COLO-206F | Colorectal | RPMI with 10% FBS |
| COLO-320 | Colorectal | RPMI with 10% FBS |
| COLO-320-DM | Colorectal | RPMI with 10% FBS |
| COLO-678 | Colorectal | RPMI with 10% FBS |
| DLD-1_BRCA2 (−/−) | Colorectal | RPMI with 10% FBS |
| DLD-1_PAR-008 | Colorectal | RPMI with 10% FBS |
| Gp2D | Colorectal | DMEM with 10% FBS and 2mM Glutamine |
| HCT-116_ARID1A | Colorectal | McCoy's 5A with 10% FBS and 2 mM Glutamine |
| (Q456/Q456) | | |
| HCT-116_KRAS | Colorectal | McCoy's 5A with 10% FBS and 2 mM Glutamine |
| (+/−) | | |
| HCT-116_PAR-007 | Colorectal | McCoy's 5A with 10% FBS and 2 mM Glutamine |
| HCT-116_PIK3A | Colorectal | McCoy's 5A with 10% FBS and 2 mM Glutamine |
| (+/−) KO mt H1047R | | |
| HCT-116_PTEN (−/−) | Colorectal | McCoy's 5A with 10% FBS and 2 mM Glutamine |
| HCT-116_TP53 (−/−) | Colorectal | McCoy's 5A with 10% FBS and 2 mM Glutamine |
| HCT-15 | Colorectal | RPMI with 10% FBS |
| HRT-18G | Colorectal | DMEM with 5% FBS |
| HT-115 | Colorectal | DMEM with 15% FBS and 2 mM Glutamine |
| HT-29 | Colorectal | McCoy's 5A with 10% FBS |
| HT55 | Colorectal | EMEM with 20% FBS, 2 mM Glutamine and 1% Non |
| | | Essential Amino Acids (NEAA) |
| LoVo | Colorectal | F12 K with 10% FBS |
| LS-411N | Colorectal | RPMI with 10% FBS |
| MDST8 | Colorectal | DMEM with 10% FBS |
| RKO | Colorectal | Eagles MEM with 10% FBS |
| SNU-1033 | Colorectal | RPMI with 10% FBS + L-glutamine (300 mg/L), 25 mM |
| | | HEPES with 25mM NaHCO3 |
| SNU-1197 | Colorectal | RPMI with 10% FBS, 25 mM HEPES and 25 mM sodium |
| | | bicarbonate |
| SNU-175 | Colorectal | RPMI with 10% FBS, 25 mM HEPES and 25 mM sodium |
| | | bicarbonate |
| SNU-283 | Colorectal | RPMI with 10% FBS |
| SNU-407 | Colorectal | RPMI with 10% FBS |
| SNU-C2B | Colorectal | RPMI with 10% FBS |
| SW1116 | Colorectal | RPMI with 10% FBS |
| SW1417 | Colorectal | RPMI with 10% FBS |
| SW480 | Colorectal | RPMI with 10% FBS |
| SW837 | Colorectal | RPMI with 10% FBS |
| COLO-684 | Endometrium | RPMI with 10% FBS |
| HEC-1 | Endometrium | EMEM with 10% FBS |
| HEC-108 | Endometrium | Eagles MEM with 15% FBS |
| HEC-1-A | Endometrium | McCoy's 5A with 10% FBS |
| HEC-265 | Endometrium | Eagles MEM with 15% FBS |
| HEC-50B | Endometrium | Eagles MEM with 15% FBS |
| JHUEM-2 | Endometrium | 1:1 RPMI, Ham's F12 with 10% FBS |
| JHUEM-3 | Endometrium | DMEM:Ham's F12 (1:1) with 10% FBS and 0.1 mM |
| | | NEAA |
| MES-SA | Endometrium | McCoy's 5A and 10% FBS |
| MFE-280 | Endometrium | 1:1 RPMI, MEMa with 20% FBS and 1× insulin- |
| | | transferrin-sodium selenite |
| MFE-296 | Endometrium | RPMI with 10% FBS |
| RL95-2 | Endometrium | DMEM F12 with 10% FBS with 0.005 mg/mL bovine |
| | | insulin |
| SK-UT-1 | Endometrium | Eagles MEM with 10% FBS and 1% NEAA |
| SNG-II | Endometrium | DMEM with 10% FBS |
| TEN | Endometrium | EMEM with 10% FBS |
| ECC10 | Gastric | RPMI with 10% FBS |
| ECC12 | Gastric | RPMI with 10% FBS |
| GSS | Gastric | RPMI with 10% FBS |
| GSU | Gastric | RPMI with 10% FBS |
| KE-97 | Gastric | RPMI with 10% FBS |
| LMSU | Gastric | Ham's F10 with 10% FBS |
| MKN1 | Gastric | RPMI with 10% FBS |
| NCC-StC-K140 | Gastric | RPMI with 10% FBS |
| NUGC-3 | Gastric | RPMI with 10% FBS |
| RERF-GC-1B | Gastric | RPMI with 10% FBS |
| SH-10-TC | Gastric | RPMI with 10% FBS |
| SNU-216 | Gastric | RPMI with 10% FBS, 25 mM HEPES and 25 mM sodium |
| | | bicarbonate |
| SNU-5 | Gastric | RPMI with 10% FBS, 25 mM HEPES and 25 mM sodium |
| | | bicarbonate |
| SNU-601 | Gastric | RPMI with 10% FBS, 25 mM HEPES and 25 mM sodium |
| | | bicarbonate |
| SNU-620 | Gastric | RPMI with 10% FBS, 25 mM HEPES and 25 mM sodium |
| | | bicarbonate |
| SNU-668 | Gastric | RPMI with 10% FBS, 25 mM HEPES and 25 mM sodium |
| | | bicarbonate |
| SNU-719 | Gastric | RPMI with 10% FBS, 25 mM HEPES and 25 mM sodium |
| | | bicarbonate |
| BICR 18 | Head and Neck | DMEM with 10% FBS with 2 mM Glutamine and 0.4 |
| | | micrograms/mL Hydrocortisone |
| BICR 22 | Head and Neck | DMEM with 10% FBS with 2 mM Glutamine and 0.4 |
| | | micrograms/mL Hydrocortisone |
| BICR 31 | Head and Neck | DMEM with 2% FBS with 2 mM Glutamine and 0.4 |
| | | micrograms/mL Hydrocortisone |
| EC-GI-10 | Head and Neck | RPMI with 10% FBS |
| FTC-238 | Head and Neck | DMEM:Ham's F12 (1:1) with 5% FBS and 2 mM |
| | | Glutamin |
| HSC-4 | Head and Neck | RPMI with 10% FBS |
| KYM-1 | Head and Neck | DMEM with 10% FBS |
| KYSE-270 | Head and Neck | 1:1 Hams F12:rpmi 1640 with 2% FBS and 2 mM |
| | | Glutamine |
| KYSE-30 | Head and Neck | RPMI with 10% FBS |
| KYSE-410 | Head and Neck | Eagles MEM with 10% FBS |
| KYSE-70 | Head and Neck | RPMI with 10% FBS |
| PE-CA-P334-cl C12 | Head and Neck | IDMEM with 10% FBS and 2 mM Glutamine |
| SCC-15 | Head and Neck | DMEM and Ham's F12 (1:) with 10% FBS and |
| | | supplements |
| TE-1 | Head and Neck | RPMI with 10% FBS |
| TE-10 | Head and Neck | RPMI with 10% FBS |
| TE-4 | Head and Neck | RPMI with 10% FBS |
| TE-5 | Head and Neck | RPMI with 10% FBS |
| TE-6 | Head and Neck | RPMI with 10% FBS |
| TE-8 | Head and Neck | RPMI with 10% FBS |
| TE-9 | Head and Neck | RPMI with 10% FBS |
| YD-15 | Head and Neck | RPMI with 10% FBS, 25 mM HEPES and 25 mM sodium |
| | | bicarbonate |
| YD-8 | Head and Neck | RPMI with 10% FBS, 25 mM HEPES and 25 mM sodium |
| | | bicarbonate |
| HLE | Liver | Eagles MEM with 10% FBS and 1% NEAA |
| HuCCT1 | Liver | RPMI with 10% FBS |
| HuH-1 | Liver | DMEM with 10% FBS |
| HuH-28 | Liver | EMEM with 10% FBS |
| HUH-6-clone5 | Liver | DMEM with 10% FBS |
| Li-7 | Liver | RPMI with 10% FBS |
| RH-41 | Liver | RPMI with 10% FBS |
| SNU-1079 | Liver | RPMI with 10% FBS, 25 mM HEPES and 25 mM sodium |
| | | bicarbonate |
| SNU-1196 | Liver | RPMI with 10% FBS, 25 mM HEPES and 25 mM sodium |
| | | bicarbonate |
| SNU-308 | Liver | RPMI with 10% FBS, 25 mM HEPES and 25 mM sodium |
| | | bicarbonate |
| SNU-478 | Liver | RPMI with 10% FBS, 25 mM HEPES and 25 mM sodium |
| | | bicarbonate |
| SNU-761 | Liver | RPMI with 10% FBS, 25 mM HEPES and 25 mM sodium |
| | | bicarbonate |
| SNU-869 | Liver | RPMI with 10% FBS, 25 mM HEPES and 25 mM sodium |
| | | bicarbonate |
| SNU-878 | Liver | RPMI with 10% FBS, 25 mM HEPES and 25 mM sodium |
| | | bicarbonate |
| BEN | Lung, NSCLC | RPMI with 10% FBS |
| CAL-12T | Lung, NSCLC | DMEM with 10% FBS |
| Calu-1 | Lung, NSCLC | McCoy's 5A with 10% FBS |
| COLO-699 | Lung, NSCLC | RPMI with 10% FBS |
| COR-L105 | Lung, NSCLC | RPMI with 10% FBS |
| COR-L23 | Lung, NSCLC | RPMI with 10% FBS |
| DV-90 | Lung, NSCLC | RPMI with 10% FBS and NEAA |
| EPLC-272H | Lung, NSCLC | RPMI with 20% FBS |
| HLC-1 | Lung, NSCLC | Ham's F12K with 10% FBS |
| HOP-62 | Lung, NSCLC | RPMI with 10% FBS |
| LC-1F | Lung, NSCLC | RPMI with 10% FBS |
| LC-1sq | Lung, NSCLC | 1:1 Hams F12:rpmi 1640 with 10% FBS and 25 mM |
| | | Hepes |
| LCLC-103H | Lung, NSCLC | RPMI with 10% FBS |
| LCLC-97TM1 | Lung, NSCLC | RPMI with 20% FBS |
| LK-2 | Lung, NSCLC | RPMI with 10% FBS |
| LOU-NH91 | Lung, NSCLC | RPMI with 20% FBS |
| LU-65 | Lung, NSCLC | RPMI with 10% FBS |
| LU-99 | Lung, NSCLC | RPMI with 10% FBS |
| LUDLU-1 | Lung, NSCLC | RPMI with 10% FBS |
| LXF-289 | Lung, NSCLC | Ham's F10 with 10% FBS |
| NCI-H460 | Lung, NSCLC | RPMI with 10% FBS |
| NCI-H820 | Lung, NSCLC | RPMI with 10% FBS |
| RERF-LC-K3 | Lung, NSCLC | RPMI with 10% FBS |
| SW1573 | Lung, NSCLC | RPMI with 10% FBS |
| T3M-10 | Lung, NSCLC | Ham's F10 with 10% FBS |
| VMRC-LCD | Lung, NSCLC | Eagles MEM with 10% FBS |
| ACC-MESO-1 | Lung, | RPMI with 10% FBS |
| | mesothelioma | |
| JL-1 | Lung, | DMEM with 20% FBS |
| | mesothelioma | |
| JU77 | Lung, | RPMI with 5% FBS |
| | mesothelioma | |
| LO68 | Lung, | RPMI with 5% FBS |
| | mesothelioma | |
| MPP-89 | Lung, | RPMI with 10% FBS |
| | mesothelioma | |
| IST-SL2 | Lung, SCLC | RPMI with 20% FBS |
| LU-134-A | Lung, SCLC | RPMI with 10% FBS |
| LU-135 | Lung, SCLC | RPMI with 10% FBS |
| LU-139 | Lung, SCLC | RPMI with 10% FBS |
| NCI-H345 | Lung, SCLC | HITES medium |
| NCI-H446 | Lung, SCLC | RPMI with 10% FBS |
| NCI-H69 | Lung, SCLC | RPMI with 10% FBS |
| SHP-77 | Lung, SCLC | RPMI with 10% FBS |
| A375 | Melanoma | DMEM with 10% FBS |
| OM | Melanoma4 | RPMI with 10% FBS |
| COLO-679 | Melanoma | RPMI with 10% FBS |
| COLO-783 | Melanoma | RPMI with 10% FBS |
| COLO-800 | Melanoma | RPMI with 10% FBS |
| COLO-818 | melanoma | RPMI with 10% FBS |
| COLO-849 | Melanoma | RPMI with 10% FBS |
| COLO-858 | Melanoma | RPMI with 10% FBS |
| HMCB | Melanoma | EMEM with 10% FBS, NEAA, 10 mM HEPES |
| | | and 1 mM sodium pyruvate (NaP) |
| Hs 294T | Melanoma | DMEM with 10% FBS |
| Hs 936.T | Melanoma | DMEM with 10% FBS |
| IGR-1 | Melanoma | DMEM with 15% FBS |
| IGR-37 | Melanoma | DMEM with 15% FBS |
| IGR-39 | Melanoma | DMEM with 15% FBS |
| IPC-298 | Melanoma | RPMI with 10% FBS |
| MEL-HO | Melanoma | RPMI with 10% FBS |
| MEL-JUSO | Melanoma | RPMI with 10% FBS |
| RVH-421 | Melanoma | RPMI with 10% FBS |
| SK-MEL-30 | Melanoma | Ham's F12 with 10% FBS |
| WM-266-4 | Melanoma | EMEM with 10% FBS and 2 mM Glutamine and 1% |
| | | NEAA and 1% NaP |
| 59M | Ovary | DMEM with 10% FBS and 2 mM Glutamine and 1 mM |
| | | NaP and 20 IU/L Bovine Insulin |
| COV434 | Ovary | DMEM with 10% FBS and 2 mM Glutamine |
| COV504 | Ovary | DMEM with 10% FBS |
| COV644 | Ovary | DMEM with 10% FBS and 2 mM Glutamine |
| JHOC-5 | Ovary | DMEM:Ham's F12 (1:1) with 10% FBS and 0.1 mM |
| | | NEAA |
| JHOM-1 | Ovary | DMEM:Ham's F12 (1:1) with 10% FBS and 0.1 mM |
| | | NEAA |
| JHOM-2B | Ovary | DMEM:Ham's F12 (1:1) with 10% FBS and 0.1 mM |
| | | NEAA |
| JHOS-4 | Ovary | DMEM:Ham's F12 (1:1) with 10% FBS and 0.1 mM |
| | | NEAA |
| KURAMOCHI | Ovary | RPMI with 10% FBS |
| MCAS | Ovary | EMEM with 20% FBS |
| OV7 | Ovary | DMEM:Ham's F12 (1:1) with 5% FBS, 2 mM Glutamine, |
| | | 0.5 pg/mL hydrocortisone and 10 μg/mL insulin |
| OVCAR-5 | Ovary | RPMI with 10% FBS |
| OVISE | Ovary | RPMI with 10% FBS |
| OVK18 | Ovary | RPMI with 10% FBS |
| OVTOKO | Ovary | RPMI with 10% FBS |
| SNU-119 | Ovary | DMEM with 10% FBS |
| BxPC-3 | Pancreas | RPMI with 10% FBS |
| CAPAN-2 | Pancreas | McCoy's 5A and 10% FBS |
| CFPAC-1 | Pancreas | Iscoves with 20% FBS |
| HPAF-II | Pancreas | Eagles MEM with 10% FBS |
| HuP-T3 | Pancreas | 1:1 DMEM, Ham's F12 with 10% FBS and 1% NEAA |
| KLM-1 | Pancreas | RPMI with 10% FBS |
| KP-2 | Pancreas | RPMI with 10% FBS |
| KP-3 | Pancreas | RPMI with 10% FBS |
| KP-4 | Pancreas | RPMI with 10% FBS |
| Panc 02.13 | Pancreas | RPMI with 15% FBS with 10 units/mL human |
| | | recombinant insulin |
| PK-1 | Pancreas | RPMI with 10% FBS |
| PSN1 | Pancreas | RPMI with 10% FBS |
| 769-P | Renal | RPMI with 10% FBS |
| 786-0 | Renal | RPMI with 10% FBS |
| A498 | Renal | Eagles MEM with 10% FBS |
| A704 | Renal | EMEM (EBSS) with 10% FBS and 2 mM Glutamine and |
| | | 1% NEAA and 1 mM NaP |
| ACHN | Renal | Eagles MEM with 10% FBS |
| CAKI-2 | Renal | McCoy's 5A with 10% FBS |
| CAL-54 | Renal | DMEM with 15% FBS and 0.4 μg/ml hydrocortisone |
| | | and 10 ng/mL EGF |
| G-401 | Renal | McCoy's 5A with 10% FBS |
| G-402 | Renal | McCoy's 5A with 10% FBS |

When cells reached expected doubling times, cells were harvested by trypsinization and single cell suspensions (500 cells per well) were seeded in black 384-well assay plates (Corning) and allowed to adhere overnight at 37° C. The following day, 0.25 μL antibody samples (0-71 nM final in screening 1 or 0-67 nM in screening 2 in 3-fold dilutions) or a medium control were added to 25 μL media per assay well and incubated for 3 days at 37° C. (except for DLD-1 and HCT 116 cell lines, for which a 120-hour assay was performed). Samples were tested as four replicates in 384-well assay plates. The viability of the cultured cells was determined in an ATPlite 1step Luminescence Assay System (Perkin Elmer, Cat no 6016739) that quantifies the presence of ATP, which is an indicator of metabolically active cells. Viability was also determined at time of treatment for samples which did not receive antibody sample. From the kit, 15 μL ATPLite suspension were added to 25 μL of media per assay well of the viability assay plate, luminescence was measured on an ultra-sensitive luminescence Envision plate reader. All data points were collected via automated processes and were subject to quality control and analyzed using Chalice viewer software (Horizon). Percentage inhibition was calculated using the formulas: If T≥V(0) than percentage inhibition=100×[1−(T−V(0))/(V−V(0))]; If T≤V(0) than percentage inhibition=100%, with T=luminescence of the test sample, V(0)=luminescence of the medium control sample on day 0 and V=luminescence of the medium control sample on day 3. Percentage viability was calculated using the formula: 100—percentage inhibition.

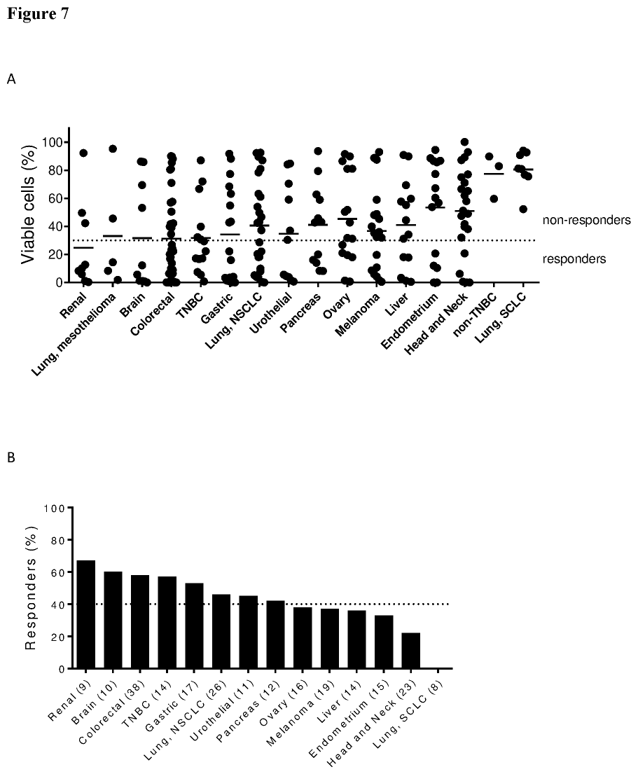

Cell lines that showed 30% tumor cell viability in presence of the mixture of IgG1-hDR5-01-G56T-E430G and IgG1-hDR5-05-E430G (i.e. >70% inhibition of tumor cell viability) were classified as responders. For all tumor types, except non-TNBC and SCLC, at least one responding cell line was identified (FIG. 7). For tumor types for which more than 5 cell lines were included in the panel, the percentage of responding cell lines was calculated (FIG. 7). In renal, colorectal, gastric, urothelial and pancreatic cancer, TNBC, NSCLC and brain tumors, more than 40% of the tested cell lines responded to treatment with the mixture of IgG1-hDR5-01-G56T-E430G and IgG1-hDR5-05-E430G. A complete overview for the individual cell lines is provided in Table 9.

Table 9: Overview of cytotoxicity in 240 human tumor cell lines. IC50, IC90 values and percentage of maximal growth inhibition from a 72-hour ATPlite assay (except for DLD-1 and HCT 116 cell lines, for which a 120-hour assay was performed), to test the cytotoxicity of the mixture of IgG1-hDR5-01-G56T-E430G and IgG1-hDR5-05-E430G. The screening was performed in two parts (#1 and #2). For cell lines tested in both experiments, the average values were calculated. A. Urothelial cancer (11 cell lines); B. Brain tumors (10 cell lines); C. non-TNBC (3 cell lines); D. TNBC (14 cell lines); E. colorectal cancer (38 cell lines); F. Endometrial cancer (15 cell lines); G. Gastric cancer (17 cell lines); H. Head and neck cancer (23 cell lines); I. Renal cell carcinoma (9 cell lines); J. Liver cancer (14 cell lines); K. NSCLC (26 cell lines); L. SCLC (8 cell lines); M. Lung mesothelioma cancer (5 cell lines); N. Melanoma (19 cell lines); 0. Ovarian cancer (16 cell lines); P. Pancreatic cancer (12 cell lines).

TABLE 9 A

| | | | | | Max |
|---|---|---|---|---|---|
| Cancer | | | IC50 | IC90 | Inhibition |
| subtype | Cell Line | Screening # | (nM) | (nM) | (%) |
| Urothelial | SW780 | average | 0.421 | 0.632 | 98.5 |
| | | screening | | | |
| | | 1 + 2 | | | |
| Urothelial | UM-UC-3 | average | 0.893 | 1.709 | 94.4 |
| | | screening | | | |
| | | 1 + 2 | | | |
| Urothelial | SCABER | average | 2.375 | 13.528 | 29.6 |
| | | screening | | | |
| | | 1 + 2 | | | |
| Urothelial | J82 | average | 34.493 | 42.991 | 15.9 |
| | | screening | | | |
| | | 1 + 2 | | | |
| Urothelial | 5637 | 2 | 0.828 | 1.607 | 99.3 |
| Urothelial | RT-112 | 2 | 3.520 | 14.684 | 96.1 |
| Urothelial | RT4 | 2 | 4.638 | 12.390 | 95.5 |
| Urothelial | TCCSUP | 2 | 1.367 | 3.023 | 69.6 |
| Urothelial | T-24 | 2 | 1.300 | 3.904 | 63.0 |
| Urothelial | HT-1197 | 2 | 0.782 | 2.354 | 40.9 |
| Urothelial | HT-1376 | 2 | 67.114 | 83.818 | 15.3 |

TABLE 9 B

| | | | | | Max |
|---|---|---|---|---|---|
| Cancer | | | IC50 | IC90 | Inhibition |
| subtype | Cell Line | Screening # | (nM) | (nM) | (%) |
| Brain | U-87 MG | 2 | 1.784 | 3.307 | 87.8 |
| Brain | A172 | 2 | 0.888 | 1.106 | 100.0 |
| Brain | SF295 | 2 | 0.764 | 0.952 | 99.2 |
| Brain | SF126 | 2 | 0.713 | 0.888 | 98.9 |
| Brain | H4 | 2 | 0.459 | 0.673 | 98.8 |
| Brain | YH-13 | 2 | 1.248 | 2.477 | 94.4 |
| Brain | DBTRG-05MG | 2 | 0.971 | 3.633 | 46.8 |
| Brain | KNS-81 | 2 | 13.008 | 16.190 | 30.6 |
| Brain | SNB-75 | 2 | 3.225 | 38.586 | 14.2 |
| Brain | NMC-G1 | 2 | 0.005 | 0.057 | 13.8 |

TABLE 9 C

| | | | | | Max |
|---|---|---|---|---|---|
| Cancer | | | IC50 | IC90 | Inhibition |
| subtype | Cell Line | Screening # | (nM) | (nM) | (%) |
| Breast. | SK-BR-3 | 2 | 1.757 | 11.583 | 40.4 |
| non-TNBC | | | | | |
| Breast. | T47D | 2 | 2.557 | 3.185 | 17.1 |
| non-TNBC | | | | | |
| Breast. | MCF7 | 2 | 19.492 | 24.390 | 10.2 |
| non-TNBC | | | | | |

TABLE 9 D

| | | | | | Max |
|---|---|---|---|---|---|
| Cancer | | | IC50 | IC90 | Inhibition |
| subtype | Cell Line | Screening # | (nM) | (nM) | (%) |
| Breast, TNBC | MDA-MB-231 | average | 0.914 | 3.941 | 60.2 |
| | | screening | | | |
| | | 1 + 2 | | | |
| Breast, TNBC | SUM159PT | 2 | 0.569 | 0.978 | 99.2 |
| Breast, TNBC | DU-4475 | 2 | 1.102 | 3.407 | 94.5 |
| Breast, TNBC | HCC1806 | 2 | 2.678 | 8.912 | 92.5 |
| Breast, TNBC | BT-549 | 2 | 1.021 | 2.176 | 83.3 |
| Breast, TNBC | BT-20 | 2 | 2.843 | 22.068 | 82.8 |
| Breast, TNBC | HCC1187 | 2 | 1.521 | 5.501 | 82.8 |
| Breast, TNBC | MDA-MB-436 | 2 | 0.762 | 0.950 | 77.7 |
| Breast, TNBC | HCC38 | 2 | 0.903 | 2.377 | 70.6 |
| Breast, TNBC | HCC70 | 2 | 18.703 | 76.051 | 69.3 |
| Breast, TNBC | HMC-1-8 | 2 | 0.714 | 1.728 | 67.7 |
| Breast, TNBC | MDA-MB-468 | 2 | 3.772 | 60.825 | 33.3 |
| Breast, TNBC | HCC1937 | 2 | 8.548 | 34.273 | 28.0 |
| Breast, TNBC | MDA-MB-453 | 2 | 0.723 | 0.900 | 13.0 |

TABLE 9 E

| | | | | | Max |
|---|---|---|---|---|---|
| Cancer | | | IC50 | IC90 | Inhibition |
| subtype | Cell Line | Screening # | (nM) | (nM) | (%) |
| Colorectal | CL-11 | 1 | 0.613 | 0.762 | 100.0 |
| Colorectal | GP2D | 1 | 0.731 | 1.442 | 100.0 |
| Colorectal | HT-115 | 1 | 2.091 | 5.179 | 100.0 |
| Colorectal | SNU-1197 | 1 | 1.063 | 1.781 | 100.0 |
| Colorectal | COLO 205 | 1 | 0.356 | 0.517 | 99.9 |
| Colorectal | COLO-206F | 1 | 0.198 | 0.456 | 99.9 |
| Colorectal | CL-34 | 1 | 0.379 | 0.801 | 99.8 |
| Colorectal | HRT-18G | 1 | 0.424 | 0.871 | 99.3 |
| Colorectal | HCT-15 | 1 | 0.800 | 2.346 | 98.8 |
| Colorectal | SNU-407 | 1 | 0.794 | 2.093 | 98.5 |
| Colorectal | MDST8 | 1 | 0.972 | 2.131 | 93.6 |
| Colorectal | COLO-201 | 1 | 0.551 | 0.764 | 93.6 |
| Colorectal | HT55 | 1 | 0.888 | 2.862 | 91.0 |
| Colorectal | SNU-175 | 1 | 1.615 | 3.653 | 90.1 |
| Colorectal | HCT-116_ARID1A | 1 | 0.198 | 0.527 | 86.2 |
| | (Q456*/Q456*) | | | | |
| Colorectal | DLD-1_PAR-008 | 1 | 0.879 | 1.095 | 83.1 |
| Colorectal | SNU-283 | 1 | 3.787 | 50.610 | 81.9 |
| Colorectal | CL-40 | 1 | 1.004 | 5.039 | 79.9 |
| Colorectal | HCT-116_KRAS (+/−) | 1 | 0.791 | 0.986 | 77.7 |
| Colorectal | SW837 | 1 | 0.952 | 3.268 | 76.6 |
| Colorectal | LOVO | 1 | 2.776 | 16.859 | 75.7 |
| Colorectal | LS-411N | 1 | 2.276 | 7.983 | 73.1 |
| Colorectal | HT-29 | 1 | 1.304 | 5.161 | 65.5 |
| Colorectal | SNU-1033 | 1 | 1.959 | 6.270 | 60.9 |
| Colorectal | SW480 | 1 | 0.853 | 1.066 | 60.2 |
| Colorectal | COLO-678 | 1 | 1.478 | 4.220 | 58.6 |
| Colorectal | DLD-1_BRCA2 (−/−) | 1 | 1.115 | 1.860 | 58.3 |
| Colorectal | HCT-116_PIK3A (+/−) | 1 | 0.353 | 0.583 | 57.8 |
| | KO mt H1047R | | | | |
| Colorectal | C2BBe1 | 1 | 2.406 | 7.635 | 49.5 |
| Colorectal | SNU-C2B | 1 | 0.840 | 1.462 | 43.0 |
| Colorectal | SW1116 | 1 | 2.873 | 3.777 | 42.4 |
| Colorectal | HCT-116_PAR-007 | 1 | 0.520 | 1.874 | 29.0 |
| Colorectal | HCT-116_TP53 (−/−) | 1 | 0.292 | 0.364 | 19.6 |
| Colorectal | SW1417 | 1 | 1.645 | 3.906 | 19.1 |
| Colorectal | RKO | 1 | 0.798 | 0.994 | 14.5 |
| Colorectal | HCT-116_PTEN (−/−) | 1 | 0.479 | 0.596 | 11.9 |
| Colorectal | COLO-320-DM | 1 | 0.559 | 4.568 | 10.1 |
| Colorectal | COLO-320 | 1 | 12.386 | 15.424 | 9.9 |

TABLE 9 F

| | | | | | Max |
|---|---|---|---|---|---|
| Cancer | | | IC50 | IC90 | Inhibition |
| subtype | Cell Line | Screening # | (nM) | (nM) | (%) |
| Endometrium | HEC-265 | 1 | 0.395 | 0.632 | 100.0 |
| Endometrium | MES-SA | 1 | 0.505 | 0.736 | 100.0 |
| Endometrium | JHUEM-2 | 1 | 0.545 | 1.297 | 89.6 |
| Endometrium | RL95-2 | 1 | 1.436 | 3.776 | 88.0 |
| Endometrium | SNG-II | 1 | 0.821 | 2.092 | 79.3 |
| Endometrium | JHUEM-3 | 1 | 5.219 | 12.041 | 46.2 |
| Endometrium | TEN | 1 | 3.875 | 14.322 | 43.2 |
| Endometrium | HEC-1-A | 1 | 0.625 | 1.120 | 39.6 |
| Endometrium | HEC-108 | 1 | 0.001 | 0.008 | 32.8 |
| Endometrium | MFE-296 | 1 | 1.648 | 2.790 | 22.7 |
| Endometrium | SK-UT-1 | 1 | 1.048 | 1.305 | 13.4 |
| Endometrium | HEC-1 | 1 | 0.109 | 0.139 | 13.2 |
| Endometrium | MFE-280 | 1 | 1.189 | 1.481 | 11.4 |
| Endometrium | COLO-684 | 1 | 129.998 | 1172.632 | 7.6 |
| Endometrium | HEC-50B | 1 | 64.087 | 79.957 | 5.5 |

TABLE 9 G

| | | | | | Max |
|---|---|---|---|---|---|
| Cancer | | | IC50 | IC90 | Inhibition |
| subtype | Cell Line | Screening # | (nM) | (nM) | (%) |
| Gastric | SNU-620 | 1 | 0.816 | 1.017 | 100.0 |
| Gastric | SNU-668 | 1 | 0.365 | 0.716 | 99.9 |
| Gastric | SNU-719 | 1 | 1.445 | 4.035 | 98.9 |
| Gastric | SNU-601 | 1 | 0.506 | 1.093 | 98.6 |
| Gastric | NUGC-3 | 1 | 0.628 | 2.832 | 96.7 |
| Gastric | GSU | 1 | 0.160 | 0.357 | 96.4 |
| Gastric | SNU-5 | 1 | 0.710 | 0.953 | 95.9 |
| Gastric | SNU-216 | 1 | 4.723 | 9.806 | 84.0 |
| Gastric | NCC-StC-K140 | 1 | 0.826 | 1.796 | 77.8 |
| Gastric | KE-97 | 1 | 0.546 | 1.044 | 57.2 |
| Gastric | LMSU | 1 | 0.999 | 3.317 | 56.6 |
| Gastric | RERF-GC-1B | 1 | 0.829 | 4.004 | 45.1 |
| Gastric | MKN1 | 1 | 0.837 | 7.523 | 36.8 |
| Gastric | SH-10-TC | 1 | 0.668 | 1.432 | 31.6 |
| Gastric | ECC12 | 1 | 5.924 | 13.083 | 22.7 |
| Gastric | GSS | 1 | 142.454 | 177.477 | 12.1 |
| Gastric | ECC10 | 1 | 141.602 | 177.867 | 8.3 |

TABLE 9 H

| | | | | | Max |
|---|---|---|---|---|---|
| Cancer | | | IC50 | IC90 | Inhibition |
| subtype | Cell Line | Screening # | (nM) | (nM) | (%) |
| Head & Neck | YD-15 | 1 | 1.355 | 3.105 | 100.0 |
| Head & Neck | TE-4 | 1 | 0.868 | 1.081 | 99.9 |
| Head & Neck | KYM-1 | 1 | 0.431 | 0.730 | 99.4 |
| Head & Neck | FTC-238 | 1 | 0.395 | 0.975 | 93.8 |
| Head & Neck | KYSE-70 | 1 | 1.129 | 1.776 | 79.2 |
| Head & Neck | TE-10 | 1 | 2.192 | 6.007 | 68.0 |
| Head & Neck | TE-6 | 1 | 1.778 | 9.554 | 61.9 |
| Head & Neck | TE-9 | 1 | 0.473 | 1.130 | 60.3 |
| Head & Neck | TE-1 | 1 | 0.879 | 3.671 | 58.3 |
| Head & Neck | BICR 31 | 1 | 1.325 | 2.637 | 52.2 |
| Head & Neck | KYSE-410 | 1 | 2.523 | 3.146 | 51.0 |
| Head & Neck | OM | 1 | 0.919 | 1.145 | 48.8 |
| Head & Neck | BICR 22 | 1 | 4.202 | 49.504 | 42.0 |
| Head & Neck | KYSE-30 | 1 | 1.691 | 7.367 | 38.0 |
| Head & Neck | SCC-15 | 1 | 3.725 | 13.942 | 34.8 |
| Head & Neck | TE-8 | 1 | 0.873 | 2.349 | 33.4 |
| Head & Neck | PE-CA-P334-cl C12 | 1 | 8.436 | 70.201 | 28.9 |
| Head & Neck | EC-GI-10 | 1 | 2.086 | 3.076 | 25.0 |
| Head & Neck | TE-5 | 1 | 2.738 | 3.423 | 23.0 |
| Head & Neck | HSC-4 | 1 | 2.406 | 3.007 | 12.8 |
| Head & Neck | YD-8 | 1 | 6.742 | 8.403 | 10.8 |
| Head & Neck | KYSE-270 | 1 | 7.125 | 34.777 | 7.1 |
| Head & Neck | BICR 18 | 1 | 142.518 | 177.495 | −0.1 |

TABLE 9 I

| | | | | | Max |
|---|---|---|---|---|---|
| Cancer | | | IC50 | IC90 | Inhibition |
| subtype | Cell Line | Screening # | (nM) | (nM) | (%) |
| Renal | A704 | 1 | 0.469 | 0.801 | 99.7 |
| Renal | CAL-54 | average | 1.502 | 3.313 | 91.7 |
| | | screening | | | |
| | | 1 + 2 | | | |
| Renal | CAKI-2 | 1 | 1.350 | 3.766 | 87.3 |
| Renal | A498 | 2 | 1.223 | 2.136 | 98.9 |
| Renal | G-401 | 2 | 0.509 | 0.795 | 94.1 |
| Renal | ACHN | 2 | 0.843 | 1.720 | 89.9 |
| Renal | 769-P | 2 | 0.957 | 1.521 | 57.7 |
| Renal | G-402 | 2 | 0.605 | 1.019 | 50.4 |
| Renal | 786-0 | 2 | 0.005 | 0.022 | 7.7 |

TABLE 9 J

| | | | | | Max |
|---|---|---|---|---|---|
| Cancer | | | IC50 | IC90 | Inhibition |
| subtype | Cell Line | Screening # | (nM) | (nM) | (%) |
| Liver | SNU-878 | 1 | 0.697 | 1.564 | 99.5 |
| Liver | SNU-308 | 1 | 1.474 | 3.454 | 98.8 |
| Liver | HuH-28 | 1 | 0.881 | 1.097 | 96.7 |
| Liver | SNU-478 | 1 | 1.530 | 6.161 | 82.2 |
| Liver | HLE | 1 | 0.199 | 1.927 | 81.9 |
| Liver | SNU-869 | 1 | 1.156 | 2.709 | 68.8 |
| Liver | Li-7 | 1 | 0.965 | 2.471 | 65.9 |
| Liver | HuCCT1 | 1 | 1.356 | 6.033 | 55.6 |
| Liver | SNU-1196 | 1 | 0.920 | 6.003 | 44.8 |
| Liver | HUH-6-clone5 | 1 | 5.985 | 66.083 | 42.3 |
| Liver | SNU-1079 | 1 | 1.928 | 12.999 | 40.3 |
| Liver | HuH-1 | 1 | 0.734 | 7.826 | 30.7 |
| Liver | RH-41 | 1 | 0.860 | 1.072 | 10.1 |
| Liver | SNU-761 | 1 | 55.150 | 134.264 | 9.1 |

TABLE 9 K

| | | Screen- | | | Max |
|---|---|---|---|---|---|
| | | ing | IC50 | IC90 | Inhibition |
| Cancer subtype | Cell Line | # | (nM) | (nM) | (%) |
| Lung. NSCLC | DV-90 | 1 | 0.229 | 2.681 | 100.0 |
| Lung. NSCLC | NCI-H820 | 1 | 0.847 | 1.055 | 99.7 |
| Lung. NSCLC | LCLC-97TM1 | 1 | 1.116 | 7.074 | 96.6 |
| Lung. NSCLC | COR-L23 | 1 | 0.682 | 1.139 | 96.0 |
| Lung. NSCLC | LOU-NH91 | 1 | 0.506 | 0.838 | 94.8 |
| Lung. NSCLC | LCLC-103H | 1 | 0.506 | 0.943 | 92.2 |
| Lung. NSCLC | T3M-10 | 1 | 0.950 | 2.097 | 90.1 |
| Lung. NSCLC | LU-99 | 1 | 1.500 | 4.226 | 81.9 |
| Lung. NSCLC | HOP-62 | 1 | 0.837 | 1.043 | 81.7 |
| Lung. NSCLC | EPLC-272H | 1 | 0.840 | 3.117 | 79.7 |
| Lung. NSCLC | LUDLU-1 | 1 | 1.458 | 6.466 | 77.9 |
| Lung. NSCLC | RERF-LC-K3 | 1 | 1.930 | 4.252 | 71.4 |
| Lung. NSCLC | LXF-289 | 1 | 2.081 | 4.894 | 62.7 |
| Lung. NSCLC | COR-L105 | 1 | 1.024 | 3.224 | 57.5 |
| Lung. NSCLC | LC-1sq | 1 | 1.313 | 8.746 | 56.6 |
| Lung. NSCLC | NCI-H460 | 1 | 0.713 | 1.959 | 53.3 |
| Lung. NSCLC | LC-1F | 1 | 1.243 | 1.712 | 50.3 |
| Lung. NSCLC | SW1573 | 1 | 0.716 | 1.441 | 46.1 |
| Lung. NSCLC | LU-65 | 1 | 0.539 | 1.277 | 38.9 |
| Lung. NSCLC | HLC-1 | 1 | 1.035 | 1.650 | 36.8 |
| Lung. NSCLC | VMRC-LCD | 1 | 0.004 | 0.004 | 21.6 |
| Lung. NSCLC | LK-2 | 1 | 1.751 | 7.833 | 19.0 |
| Lung. NSCLC | Calu-1 | 1 | 2.347 | 3.394 | 13.0 |
| Lung. NSCLC | CAL-12T | 1 | 4.940 | 13.965 | 10.4 |
| Lung. NSCLC | COLO-699 | 1 | 2.436 | 3.038 | 7.7 |
| Lung. NSCLC | BEN | 1 | 0.548 | 1.235 | 6.3 |

TABLE 9 L

| | | Screen- | | | Max |
|---|---|---|---|---|---|
| | | ing | IC50 | IC90 | Inhibition |
| Cancer subtype | Cell Line | # | (nM) | (nM) | (%) |
| Lung, SCLC | LU-134-A | 1 | 1.365 | 1.700 | 47.7 |
| Lung, SCLC | IST-SL2 | 1 | 0.791 | 0.985 | 24.4 |
| Lung, SCLC | NCI-H69 | 1 | 142.492 | 177.477 | 23.2 |
| Lung, SCLC | LU-139 | 1 | 67.092 | 87.557 | 18.7 |
| Lung, SCLC | SHP-77 | 1 | 142.503 | 177.505 | 9.2 |
| Lung, SCLC | NCI-H345 | 1 | 69.543 | 86.690 | 7.4 |
| Lung, SCLC | NCI-H446 | 1 | 13.913 | 17.323 | 7.3 |
| Lung, SCLC | LU-135 | 1 | 13.041 | 16.245 | 6.0 |

TABLE 9 M

| | | Screen- | | | Max |
|---|---|---|---|---|---|
| | | ing | IC50 | IC90 | Inhibition |
| Cancer subtype | Cell Line | # | (nM) | (nM) | (%) |
| Lung, | LO68 | 1 | 0.729 | 0.907 | 98.1 |
| mesothelioma | | | | | |
| Lung, | JL-1 | 1 | 0.690 | 2.478 | 91.6 |
| mesothelioma | | | | | |
| Lung, | JU77 | 1 | 1.001 | 3.329 | 85.6 |
| mesothelioma | | | | | |
| Lung, | MPP-89 | 1 | 0.650 | 1.344 | 54.4 |
| mesothelioma | | | | | |
| Lung, | ACC-MESO-1 | 1 | 95.154 | 129.109 | 4.7 |
| mesothelioma | | | | | |

TABLE 9 N

| | | | | | Max |
|---|---|---|---|---|---|
| Cancer | | | IC50 | IC90 | Inhibition |
| subtype | Cell Line | Screening # | (nM) | (nM) | (%) |
| Melanoma | COLO-679 | 1 | 0.517 | 0.698 | 99.5 |
| Melanoma | COLO-783 | 1 | 0.494 | 0.726 | 98.2 |
| Melanoma | COLO-800 | 1 | 0.352 | 0.614 | 95.7 |
| Melanoma | Hs 294T | 1 | 0.558 | 1.037 | 94.1 |
| Melanoma | RVH-421 | 1 | 0.539 | 0.901 | 91.3 |
| Melanoma | MEL-HO | 1 | 0.710 | 1.117 | 89.2 |
| Melanoma | WM-266-4 | 1 | 0.887 | 2.708 | 80.3 |
| Melanoma | COLO-858 | 1 | 0.405 | 0.751 | 68.1 |
| Melanoma | MEL-JUSO | 1 | 0.862 | 1.830 | 67.6 |
| Melanoma | COLO-818 | 1 | 0.708 | 1.283 | 64.8 |
| Melanoma | IGR-39 | 1 | 0.415 | 1.241 | 63.9 |
| Melanoma | IGR-1 | 1 | 0.769 | 0.957 | 60.1 |
| Melanoma | IGR-37 | 1 | 1.926 | 8.412 | 54.7 |
| Melanoma | COLO-849 | 1 | 1.143 | 2.495 | 51.9 |
| Melanoma | A375 | 1 | 0.995 | 3.648 | 51.2 |
| Melanoma | Hs 936.T | 1 | 1.065 | 2.334 | 41.0 |
| Melanoma | SK-MEL-30 | 1 | 1.669 | 2.078 | 12.5 |
| Melanoma | IPC-298 | 1 | 0.894 | 2.103 | 11.2 |
| Melanoma | HMCB | 1 | 142.446 | 177.447 | 7.0 |

TABLE 9 O

| | | | | | Max |
|---|---|---|---|---|---|
| Cancer | | | IC50 | IC90 | Inhibition |
| subtype | Cell Line | Screening # | (nM) | (nM) | (%) |
| Ovary | SNU-119 | 1 | 0.670 | 1.349 | 99.3 |
| Ovary | 59M | 1 | 0.802 | 3.287 | 98.6 |
| Ovary | JHOM-2B | 1 | 5.748 | 59.864 | 82.0 |
| Ovary | COV434 | 1 | 0.806 | 2.798 | 80.4 |
| Ovary | OVCAR-5 | 1 | 1.938 | 5.790 | 75.6 |
| Ovary | OVK18 | 1 | 0.698 | 1.158 | 73.2 |
| Ovary | JHOM-1 | 1 | 1.070 | 3.442 | 68.8 |
| Ovary | COV644 | 1 | 1.451 | 7.283 | 68.2 |
| Ovary | MCAS | 1 | 4.136 | 16.351 | 57.2 |
| Ovary | JHOS-4 | 1 | 3.073 | 14.867 | 49.6 |
| Ovary | OV7 | 1 | 3.232 | 43.214 | 48.3 |
| Ovary | COV504 | 1 | 0.070 | 0.421 | 19.0 |
| Ovary | OVTOKO | 1 | 1.515 | 1.886 | 18.9 |
| Ovary | OVISE | 1 | 43.822 | 69.787 | 13.5 |
| Ovary | KURAMOCHI | 1 | 3.578 | 9.077 | 10.1 |
| Ovary | JHOC-5 | 1 | 142.464 | 177.482 | 8.5 |

TABLE 9 P

| | | | | | Max |
|---|---|---|---|---|---|
| Cancer | | | IC50 | IC90 | Inhibition |
| subtype | Cell Line | Screening # | (nM) | (nM) | (%) |
| Pancreas | HuP-T3 | 1 | 0.645 | 2.367 | 91.8 |
| Pancreas | PSN1 | 1 | 0.621 | 0.986 | 91.6 |
| Pancreas | Panc 02.13 | 1 | 1.745 | 10.676 | 85.9 |
| Pancreas | BxPC-3 | 1 | 0.387 | 0.709 | 83.9 |
| Pancreas | KP-4 | 1 | 1.300 | 4.598 | 80.0 |
| Pancreas | CFPAC-1 | 1 | 2.393 | 16.400 | 57.2 |
| Pancreas | HPAF-II | 1 | 3.557 | 9.022 | 56.9 |
| Pancreas | KP-2 | 1 | 2.853 | 8.113 | 54.2 |
| Pancreas | KLM-1 | 1 | 1.643 | 3.408 | 41.0 |
| Pancreas | KP-3 | 1 | 0.769 | 2.378 | 37.3 |
| Pancreas | CAPAN-2 | 1 | 3.012 | 3.752 | 20.6 |
| Pancreas | PK-1 | 1 | 14.064 | 17.516 | 6.4 |

### Example 9: Viability Screening Using a Human Cancer Cell Line Panel: Haematological Malignancies

The cytotoxic capacity of the mixture of IgG1-hDR5-01-G56T-E430G and IgG1-hDR5-05-E430G was explored in a panel of 45 cell lines representing different haematological malignancies (Table 8). The screening was performed at Horizon Discovery Ltd. Frozen cells were thawed and expanded in growth media.

TABLE 10
Culturing conditions cell lines haematological malignancies at Horizon Discovery Ltd.

| | Cancer | | |
|---|---|---|---|
| | type | Cell line | Culture Medium |
| 1 | AML | EOL-1 | RPMI (ThermoFisher, Gibco; Cat no 21875-091 |
| | | | with 10% FBS (ThermoFisher, Gibco; Cat no 10270106) |
| 2 | | HL-60 | IMDM (ThermoFisher, Gibco; Cat no 12440-061) |
| | | | with 20 % FBS |
| 3 | | KASUMI-1 | RPMI with 10% FBS |
| 4 | | KG-1 | IMDM with 20% FBS |
| 5 | | ML-1 | RPMI with 10% FBS |
| 6 | | MOLM-13 | RPMI with 10% FBS |
| 7 | | MV-4-11 | IMDM with 10% FBS |
| 8 | | THP-1 | RPMI with 10% FBS and 0.05 mM 2-mercaptoethanol |
| | | | (Sigma, Cat no M3148) |
| 9 | | KG-1a | IMDM with 20% FBS |
| 10 | | NOMO-1 | RPMI with 10% FBS |
| 11 | | HEL 92.1.7 | RPMI with 10% FBS |
| 1 | DLBCL | A3/KAW | RPMI with 10% FBS |
| 2 | | DB | RPMI with 10% FBS |
| 3 | | DOHH-2 | RPMI with 10% FBS |
| 4 | | OCI-Ly10 | IMDM with 20% FBS |
| 5 | | OCI-Ly19 | MEM-alpha (ThermoFisher, Gibco; Cat no 22571-020) |
| | | | with 20% FBS |
| 6 | | OCI-Ly7 | IMDM with 20% FBS |
| 7 | | Pfeiffer | RPMI with 10% FBS |
| 8 | | SU-DHL-10 | RPMI with 10% FBS |
| 9 | | SU-DHL-4 | RPMI with 10% FBS |
| 10 | | SU-DHL-5 | RPMI with 10% FBS |
| 11 | | SU-DHL-6 | RPMI with 10% FBS |
| 12 | | SU-DHL-8 | RPMI with 10% FBS |
| 13 | | Toledo | RPMI with 10% FBS |
| 14 | | U-2932 | RPMI with 10% FBS |
| 1 | FL | SC-1 | RPMI with 10% FBS |
| 2 | | WSU-NHL | RPMI with 10% FBS |
| 1 | MM | U266B1 | RPMI with 15% FBS |
| 2 | | KMS-12-BM | RPMI with 10% FBS |
| 3 | | L-363 | RPMI with 15% FBS |
| 4 | | LP-1 | IMDM with 20% FBS |
| 5 | | KMS-11 | RPMI with 10% FBS |
| 6 | | KMS-26 | RPMI with 10% FBS |
| 7 | | MM.1R | RPMI with 10% FBS |
| 8 | | MM.1S | RPMI with 10% FBS |
| 9 | | NCI-H929 | RPMI with 10% FBS and 0.05 mM 2-mercaptoethanol |
| 10 | | RPMI-8226 | RPMI with 10% FBS |
| 1 | MCL | GRANTA-519 | DMEM (ThermoFisher, Gibco; Cat no 41966-029) |
| | | | with 10% FBS |
| 2 | | REC-1 | RPMI with 10% FBS |
| 3 | | JVM-2 | RPMI with 10% FBS |
| 4 | | Jeko-1 | RPMI with 20% FBS |
| 5 | | MAVER-1 | RPMI with 10% FBS |
| 6 | | Mino | RPMI with 15% FBS |
| 7 | | JVM-13 | RPMI with 10% FBS |
| 8 | | Z-138 | IMDM with 10% FBS |

72h-viability assays were performed using the CellTiter-Glo proliferation assay in the presence of pooled complement human serum. Briefly, cell lines that have been preserved in liquid nitrogen were thawed and expanded in growth media. Once cells reached expected doubling times, cells were seeded in growth media in black 384-well tissue culture treated plates at 500-1,500 cells per well. Cells were briefly centrifuged in assay plates and incubated at 37° C. 5% CO₂ for 24 hours before treatment. At the time of treatment, a set of assay plates, which did not receive treatment, were collected and viability was determined in a CellTiter-Glo (CTG) 2.0 assay (Promega, Cat no G9243) that quantifies the presence of ATP. These T_{zero} (T₀) plates were read using ultra-sensitive luminescence on Envision plate readers (Perkin Elmer). Remaining assay plates were dosed with test agents using Echo 555 acoustic dispensers (Labcyte). Pooled complement human serum (Innovative Research) was added to assay plates to a final concentration of 20%. Assay plates were incubated with compound for 72 hours and were then analyzed using CTG 2.0.

All data points were collected via automated processes and were subject to quality control and analyzed using Chalice software (Horizon). Assay plates were accepted if they passed the following quality control standards: relative raw values consistent throughout the entire experiment, Z-factor scores greater than 0.6 and untreated/vehicle controls showing consistent behavior on the plate.

Percentage inhibition was calculated using the formulas: If T≥V₀ then percentage inhibition=100×[1−(T−V₀)/(V−V₀)]; If T≤V₀ then percentage inhibition=100%, with T=luminescence of the test sample, V=luminescence of the untreated control sample on day 3, and V₀=luminescence of the untreated control sample at time zero (T₀ plates). Percentage viability was calculated using the formula: 100−percentage inhibition.

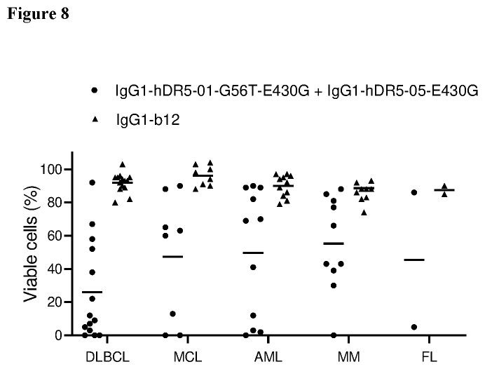

A panel of 45 cell lines was exposed to increasing concentrations of the mixture of IgG1-hDR5-01-G56T-E430G+IgG1-hDR5-05-E430G and the maximum inhibitory effect observed on cell growth was used as an indicator for their sensibility to the antibody mixture. Cell lines were grouped according to their pathological origin into five disease groups: diffuse large B-cell lymphoma (DLBCL), mantle cell lymphoma (MCL), follicular lymphoma (FL), acute myeloid leukemia (AML) and multiple myeloma (MM). Incubation for 72 hours with the mixture of IgG1-hDR5-01-G56T-E430G+IgG1-hDR5-05-E430G reduced average cell viability in all tested disease groups in comparison to control antibody IgG1-b12 (FIG. 8).

TABLE 11
Overview of cytotoxicity of the mixture of IgG1-hDR5-01-
G56T-E430G and IgG1-hDR5-05-E430G in 45 cell lines
representing different haematological malignancies. IC50
and maximal effect (percentage growth inhibition) from a
72 h CellTiter-Glo proliferation assay in the presence of
pooled complement human serum.

| | Haematological | | IC50 | Max Effect |
|---|---|---|---|---|
| | malignancy | Cell line | (μM) | (% inhibition) |
| 1 | AML | EOL-1 | 0.0006 | 97 |
| 2 | | HL-60 | 0.0214 | 31 |
| 3 | | KASUMI-1 | 0.0009 | 59 |
| 4 | | KG-1 | 0.1115 | 10 |
| 5 | | KG-1a | 0.2680 | 11 |
| 6 | | ML-1 | 0.0012 | 98 |
| 7 | | MOLM-13 | 0.0006 | 100 |
| 8 | | MV-4-11 | 0.0014 | 88 |
| 9 | | NOMO-1 | 0.0092 | 18 |
| 10 | | THP-1 | 0.0394 | 30 |
| 11 | | HEL 92.1.7 | 0.0256 | 11 |
| 1 | DLBCL | A3/KAW | 0.0089 | 93 |
| 2 | | DB | 0.0012 | 33 |
| 3 | | DOHH-2 | 0.0007 | 100 |
| 4 | | OCI-Ly10 | 0.0007 | 78 |
| 5 | | OCI-Ly19 | 0.0008 | 100 |
| 6 | | OCI-Ly7 | 0.0014 | 91 |
| 7 | | Pfeiffer | 0.0020 | 42 |
| 8 | | SU-DHL-10 | 0.0001 | 8 |
| 9 | | SU-DHL4 | 0.0005 | 97 |
| 10 | | SU-DHL-5 | 0.0009 | 95 |
| 11 | | SU-DHL-6 | 0.0020 | 88 |
| 12 | | SU-DHL-8 | 0.0011 | 100 |
| 13 | | Toledo | 0.0014 | 62 |
| 14 | | U-2932 | 0.0015 | 48 |
| 1 | FL | SC-1 | 0.2677 | 14 |
| 2 | | WSU-NHL | 0.0013 | 95 |
| 1 | MM | KMS-12-BM | 0.0017 | 57 |
| 2 | | L-363 | 0.0047 | 61 |
| 3 | | LP-1 | 0.0430 | 12 |
| 4 | | U266B1 | 0.1305 | 15 |
| 5 | | KM5-11 | 0.0014 | 23 |
| 6 | | KM5-26 | 0.0012 | 19 |
| 7 | | MM.1R | 0.0022 | 70 |
| 8 | | MM.1S | 0.0017 | 34 |
| 9 | | NCI-H929 | 0.0013 | 100 |
| 10 | | RPMI-8226 | 0.0025 | 57 |
| 1 | MCL | GRANTA-519 | 0.0008 | 35 |
| 2 | | Jeko-1 | 0.0006 | 100 |
| 3 | | JVM-13 | 0.0035 | 40 |
| 4 | | JVM-2 | 0.2679 | 12 |
| 5 | | MAVER-1 | 0.0029 | 10 |
| 6 | | Mino | 0.0010 | 100 |
| 7 | | REC-1 | 0.0009 | 37 |
| 8 | | Z-138 | 0.0010 | 87 |

### Example 10: In Vivo Efficacy Using Cell Line-Derived Xenograft Models

The in vivo therapeutic activity of the mixture of IgG1-hDR5-01-G56T-E430G and IgG1-hDR5-05-E430G was tested in xenograft tumor models derived from established human tumor cell lines (CDX) representing different solid tumor indications (Table 12). Immunodeficient 7-10 weeks old female CB17-SCID (C.B-17/IcrHan®Hsd-Prkdcscid, Harlan), BALB/c athymic nude mice (Shanghai Laboratory Animal Center, China) or NOD/SCID mice (Beijing HFK Bioscience) were used in the CDX studies. Earmarks were placed for mouse identification. Tumors were induced by subcutaneous injection of 100-200 μL tumor cell suspension containing three to ten million cells in the flank of the mouse.

TABLE 12
Cell lines used for CDX models (solid tumor inications).

| | | | | No of cells | |
|---|---|---|---|---|---|
| Name | Tumor origin | Source | Cat no. | inoculated¹ | Study performed by |
| COLO 205 | Colorectal | ATCC | CCL-222 | 3 × 10⁶ | Genmab |
| | cancer | | | | |
| HCT-15 | Colorectal | Crown Bioscience | | 5 × 10⁶ | Crown Bioscience Inc. |
| | cancer | | | | (Taicang) |
| HCT 116 | Colorectal | ATCC | CCL-247 | 5 × 10⁶ | Genmab |
| | cancer | | | | |
| SW480 | Colorectal | Crown Bioscience | | 1 × 10⁷ | Crown Bioscience Inc. |
| | cancer | | | | (Taicang) |
| BxPC-3 | Pancreatic | ATCC | CRL-1687 | 5 × 10⁶ | Genmab |
| | cancer | | | | |
| PANC-1 | Pancreatic | ATCC | CRL-1469 | 5 × 10⁶ | Genmab |
| | cancer | | | | |
| A375 | Melanoma | ATCC | CRL-1619 | 5 × 10⁶ | Genmab |
| SNU-5 | Gastric cancer | Crown Bioscience | | 1 × 10⁷ | Crown Bioscience Inc. |
| | | | | | (Taicang) |
| SK-MES-1 | Squamous cell | Crown Bioscience | | 5 × 10⁶ | Crown Bioscience Inc. |
| | lung carcinoma | | | | (Taicang) |

¹All cell lines were harvested in log-phase (at a confluence of approximately 70%).

Mice were divided into groups of 6-8 mice each, with equal tumor size distribution (average and variance). Mice were injected intraperitoneally (i.p.) or intravenously (i.v.) with 0.1 mL test solution per mouse, according to the specific schedules mentioned below. In most studies, the body weight of the mice was monitored twice weekly, including on the day of treatment. Mice were observed at least twice a week for clinical signs of illness. Tumor volumes were measured at least twice a week using a digital caliper (PLEXX). Tumor volumes (mm³) were calculated as follows: tumor volume=0.52×(length)×(width)². Statistical differences in median tumor volumes were compared between treatment groups using Mann Whitney test on the last day treatment groups were complete using graphpad prism software. Mantel-Cox analysis of Kaplan-Meier curves was performed to analyse statistical differences in progression-free survival time with a general tumor size cut-off of 500 mm³ using IBM SPSS statistics.

The experiments were ended for individual mice when the tumor size exceeded 1.5 cm³, the tumor showed ulceration, in case of serious clinical illness, when the tumor growth blocked the movement of the mouse, or when tumor growth assessment had been completed.

### Different Dosing Regimens in HCT-15 CRC CDX Model

The in vivo anti-tumor efficacy of the mixture of IgG1-hDR5-01-G56T-E430G and IgG1-hDR5-05-E430G at different dosing regimens with an equal cumulative end dose of 1.5 mg/kg was studied in the subcutaneous HCT-15 colorectal cancer xenograft model. Cells were injected in a volume of 100 μL PBS into the flank of 7-9 weeks old female BALB/c athymic nude mice. Mice were assigned to groups using randomized block design and treatments were started when the mean tumor size reached on average 161 mm³ (8 mice per group). Mice were treated on day 11 after tumor cell inoculation by i.v. injection of 10 μL test item per gram body weight; every 7 days for 2 rounds (Q7Dx2) with 0.075 mg/mL (0.75 mg/kg); every 7 days for 3 rounds (Q7Dx3) with 0.05 mg/mL (0.5 mg/kg) or on day 0, 3, 7, 10, 14 and 21 with 0.025 mg/mL (0.25 mg/kg) by i.v. injection. Furthermore a high dose with 1 mg/mL of the mixture of IgG1-hDR5-01-G56T-E430G and IgG1-hDR5-05-E430G (10 mg/kg) dosed Q7Dx2 was included and mice in the control group were treated with 0.05 mg/mL (0.5 mg/kg) IgG1-b12.

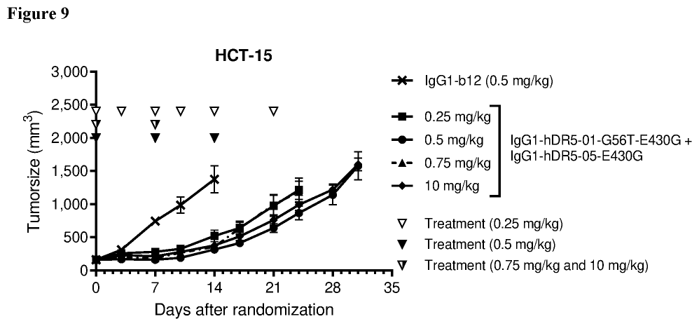

FIG. 9 shows mean tumor volumes per treatment group. Statistical analysis on the last day that all groups treated with the mixture of IgG1-hDR5-01-G56T-E430G and IgG1-hDR5-05-E430G were intact (day 24) showed that tumor growth inhibition was not significantly different when a cumulative dose of 1.5 mg/kg was given using different dosing regimens; 6×0.25 mg/kg, 3×0.5 mg/kg or 2×0.75 mg/kg. Furthermore, increasing the cumulative dose from 1.5 mg/kg to 10 mg/kg did not result in significantly increased in anti-tumor activity. These data demonstrate that frequent treatment with a low dose of 0.25 mg/kg Hx-DR5-01/05 or less frequent with a high dose of 0.5 mg/kg or 0.75 mg/kg Hx-DR5-01/05 gave a comparable anti-tumor effect in vivo.

### In Vivo Anti-Tumor Efficacy in Different CDX Models

The in vivo anti-tumor efficacy of different doses of the mixture of IgG1-hDR5-01-G56T-E430G and IgG1-hDR5-05-E430G was evaluated in subcutaneous CDX models. In these studies, a mixture of human/mouse chimeric HexaBody molecules containing a K409R or F405L mutation (IgG1-DR5-01-K409R-E430G+IgG1-DR5-05-F405L-E430G) was used as surrogate for of IgG1-hDR5-01-G56T-E430G and IgG1-hDR5-05-E430G, which is known to show functional comparability.

### COLO 205 Colorectal Cancer Xenograft Model

COLO 205 cells were injected in a volume of 200 μL PBS into the flank of 6-11 weeks old female CB17-SCID mice. Mice were assigned into groups using randomized block design and treatments were started when the mean tumor size reached ˜400 mm³ (8 mice per group). Mice were treated once by i.v. injection of 40 μg (2 mg/kg), 10 μg (0.5 mg/kg) or 2 μg (0.1 mg/kg) antibody in 100 μL PBS on day 10. Mice in the control group were treated with 40 μg (2 mg/kg) IgG1-b12.

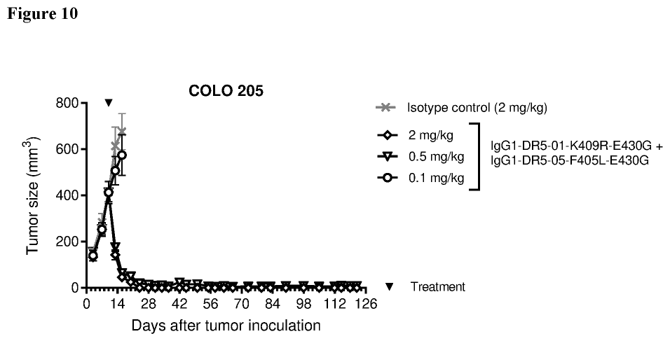

FIG. 10 shows mean tumor volumes per treatment group. Treatment with a single dose of 0.5 mg/kg or 2 mg/kg of the mixture IgG1-DR5-01-K409R-E430G+IgG1-DR5-05-F405L-E430G resulted in complete tumor regression, with no tumor recurrence until the study was stopped on day 126. At 0.1 mg/kg, the mixture IgG1-DR5-01-K409R-E430G+IgG1-DR5-05-F405L-E430G showed anti-tumor activity. These data demonstrate that the tested surrogate for the mixture of IgG1-DR5-01-G56T-E430G+IgG1-DR5-05-E430G inhibited tumor growth at 0.5 mg/kg and 2 mg/kg kg in a COLO 205 human colorectal cancer xenograft model.

### HCT-15 Colorectal Cancer Xenograft Model

HCT-15 cells were injected in a volume of 100 μL PBS into the flank of 7-9 weeks old female BALB/c athymic nude mice. Mice were assigned into groups using randomized block design and treatments were started when the mean tumor size reached on average 186 mm3 (8 mice per group). Mice were treated Q7Dx2 by i.v. injection of 10 μL test item per gram body weight; 1 mg/mL (10 mg/kg), 0.2 mg/mL (2 mg/kg) or 0.05 mg/mL (0.5 mg/kg) starting with the first dose on day 11. Mice in the control group were treated with 1 mg/mL (10 mg/kg) IgG1-b12.

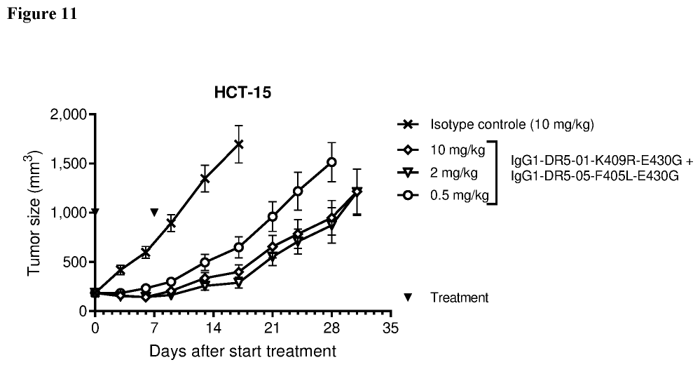

FIG. 11 shows mean tumor volumes per treatment group. All tested doses of the mixture IgG1-DR5-01-K409R-E430G+IgG1-DR5-05-F405L-E430G significantly inhibited tumor growth compared to the negative control IgG1-b12 (Mann Whitney test; 10 mg/kg p<0.0003; 2 mg/kg p<0.0002; 0.5 mg/kg p<0.0011). These data indicate that the tested surrogate for the mixture of IgG1-DR5-01-G56T-E430G+IgG1-DR5-05-E430G inhibited tumor growth at 0.5 mg/kg, 2 mg/kg and 10 mg/kg in an in vivo xenograft model with HCT-15 human colorectal cancer cells.

### SW480 Colorectal Cancer Xenograft Model

SW480 cells were injected in a volume of 200 μL PBS with Matrigel (1:1) into the flank of 6-8 weeks old female NOD/SCID mice. Mice were assigned into groups using randomized block design and treatments were started when the mean tumor size reached on average 175 mm³ (8 mice per group). Mice were treated Q7Dx2 by i.v. injection of 10 μL test item per gram body weight; 1 mg/mL (10 mg/kg), 0.2 mg/mL (2 mg/kg) or 0.05 mg/mL (0.5 mg/kg) starting with the first dose on day 10. Mice in the control group were treated with 1 mg/mL (10 mg/kg) IgG1-b12.

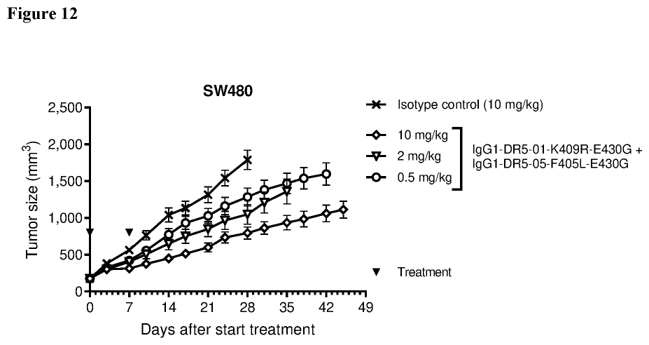

FIG. 12 shows mean tumor volumes per treatment group. Statistical analysis on the last day that all groups were intact (day 28 after start treatment) showed that all tested doses of the mixture IgG1-DR5-01-K409R-E430G+IgG1-DR5-05-F405L-E430G significantly inhibited tumor growth compared to the negative control IgG1-b12 (Mann Whitney test; 10 mg/kg p<0.0003; 2 mg/kg p<0.0047; 0.5 mg/kg p<0.0281). These data indicate that the tested surrogate for the mixture of IgG1-DR5-01-G56T-E430G+IgG1-DR5-05-E430G inhibited tumor growth at 0.5 mg/kg, 2 mg/kg and 10 mg/kg in an in vivo SW480 human colorectal cancer xenograft model.

### BxPC-3 Pancreatic Cancer Xenograft Model

BxPC-3 cells were injected in a volume of 100 μL PBS into the flank of 6-11 weeks old female CB17-SCID mice. Mice were assigned into groups using randomized block design and treatments were started when the mean tumor size reached ˜250 mm³ (8 mice per group). Mice were treated Q7Dx2 by i.v. injection of 200 μg (10 mg/kg), 40 μg (2 mg/kg) or 10 μg (0.5 mg/kg) antibody in 200 μL PBS on day 20. Mice in the control group were treated with 200 μg (10 mg/kg) IgG1-b12.

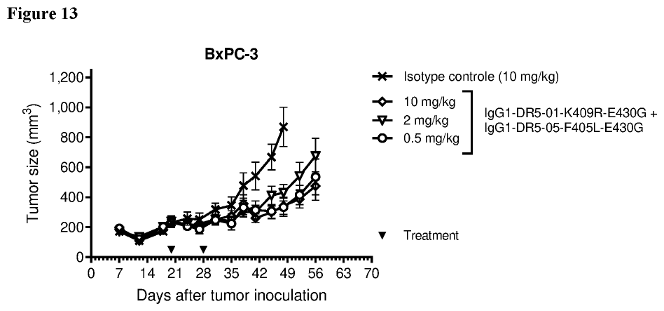

FIG. 13 shows mean tumor volumes per treatment group. Statistical analysis on the last day that all groups were intact (day 48) showed significant tumor growth inhibition at all tested doses of the mixture IgG1-DR5-01-K409R-E430G+IgG1-DR5-05-F405L-E430G compared to the negative control IgG1-b12 (Mann Whitney test; 10 mg/kg p<0.0070; 2 mg/kg p<0.0281; 0.5 mg/kg p<0.0104). These data indicate that the tested surrogate for the mixture of IgG1-DR5-01-G56T-E430G+IgG1-DR5-05-E430G inhibited tumor growth at 0.5 mg/kg, 2 mg/kg and 10 mg/kg in an in vivo BxPC-3 human pancreatic cancer xenograft model.

### PANC-1 Pancreatic Cancer Xenograft Model

PANC-1 cells were injected in a volume of 100 μL PBS into the flank of 6-11 weeks old female CB17-SCID mice. Mice were assigned into groups using randomized block design and treatments were started when the mean tumor size reached ˜250 mm³ (8 mice per group). Mice were treated once by i.v. injection of 200 μg (10 mg/kg), 40 μg (2 mg/kg) or 10 μg (0.5 mg/kg) antibody in 200 μL PBS on day 13. Mice in the control group were treated with 200 μg (10 mg/kg) IgG1-b12.

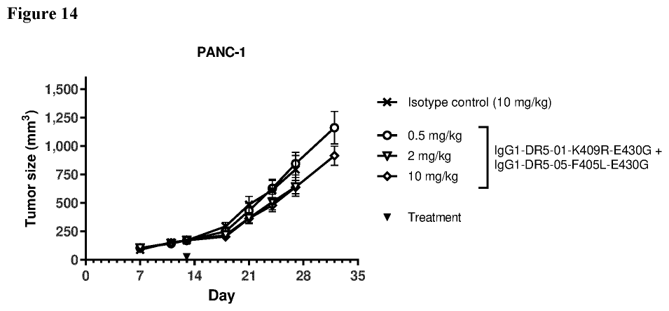

FIG. 14 shows mean tumor volumes per treatment group. No anti-tumor effect of the mixture of IgG1-DR5-01-K409R-E430G+IgG1-DR5-05-F405L-E430G was observed at any dose level.

### A375 Melanoma Xenograft Model

A375 cells were injected in a volume of 100 μL PBS into the flank of 6-11 weeks old female CB17-SCID mice. Mice were assigned into groups using randomized block design and treatments were started when the mean tumor size reached ˜250 mm³ (8 mice per group). Mice were treated Q7Dx2 by i.v. injection of 200 μg (10 mg/kg), 40 μg (2 mg/kg) or 10 μg (0.5 mg/kg) antibody in 200 μL PBS on day 19. Mice in the control group were treated with 200 μg (10 mg/kg) IgG1-b12.

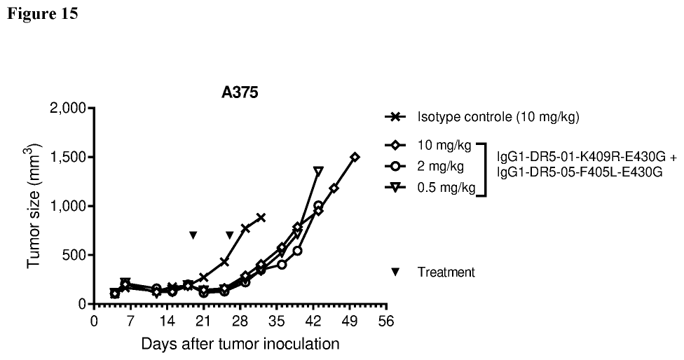

FIG. 15 shows median tumor volumes per treatment group. Statistical analysis on the last day that all groups were intact (day 29) showed significant tumor growth inhibition at all tested doses of the mixture IgG1-DR5-01-K409R-E430G+IgG1-DR5-05-F405L-E430G compared to the negative control IgG1-b12 (Mann Whitney test; 10 mg/kg p<0.0006; 2 mg/kg p<0.0006; 0.5 mg/kg p<0.0047). These data indicate that the tested surrogate for the mixture of IgG1-DR5-01-G56T-E430G+IgG1-DR5-05-E430G inhibited tumor growth at 0.5 mg/kg, 2 mg/kg and 10 mg/kg in an in vivo A375 melanoma xenograft model.

### SNU-5 Gastric Cancer Xenograft Model

SNU-5 cells were injected in a volume of 200 μL PBS with Matrigel (1:1) into the flank of 6-8 weeks old female CB17-SCID mice. Mice were assigned into groups using randomized block design and treatments were started when the mean tumor size reached on average 169 mm³ (8 mice per group). Mice were treated Q7Dx2 by i.v. injection of 10 μL test item per gram body weight; 1 mg/mL (10 mg/kg), 0.2 mg/mL (2 mg/kg) or 0.05 mg/mL (0.5 mg/kg) starting with the first dose on day 8. Mice in the control group were treated with 1 mg/mL (10 mg/kg) IgG1-b12.

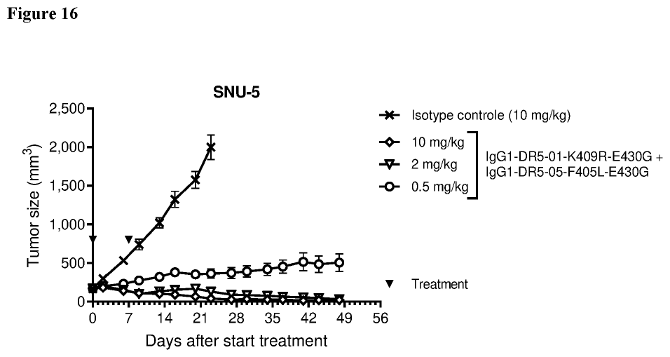

FIG. 16 shows mean tumor volumes per treatment group. Statistical analysis on the last day that all groups were intact (day 23 after start treatment) showed significant tumor growth inhibition at all tested doses of the mixture IgG1-DR5-01-K409R-E430G+IgG1-DR5-05-F405L-E430G compared to the control IgG1-b12 (Mann Whitney test; p<0.0002 for all tested doses). These data indicate that the tested surrogate for the mixture of IgG1-DR5-01-G56T-E430G+IgG1-DR5-05-E430G inhibited tumor growth at growth at 0.5 mg/kg, 2 mg/kg and 10 mg/kg in an in vivo SNU-5 human gastric cancer xenograft model.

### SK-MES-1 NSCLC Xenograft Model

SK-MES-1 cells were injected in a volume of 100 μL PBS into the flank of 7-9 weeks old female BALB/c athymic nude mice. Mice were assigned into groups using randomized block design and treatments were started when the mean tumor size reached on average 161 mm³ (8 mice per group). Mice were treated Q7Dx2 by i.v. injection of 10 μL test item per gram body weight; 1 mg/mL (10 mg/kg), 0.2 mg/mL (2 mg/kg) or 0.05 mg/mL (0.5 mg/kg) starting with the first dose on day 21. Mice in the control group were treated with 1 mg/mL (10 mg/kg) IgG1-b12.

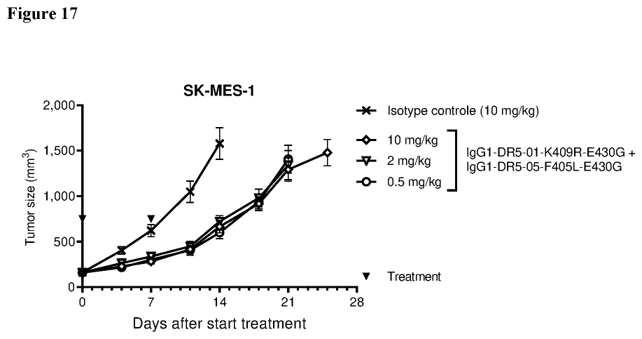

FIG. 17 shows mean tumor volumes per treatment group. Statistical analysis on the last day that all groups were intact (day 14 after start treatment) showed significant tumor growth inhibition at all tested doses of the mixture IgG1-DR5-01-K409R-E430G+IgG1-DR5-05-F405L-E430G compared to the negative control IgG1-b12 (Mann Whitney test; 10 mg/kg p<0.0012; 2 mg/kg p<0.0006; 0.5 mg/kg p<0.0003). These data indicate that the tested surrogate for the mixture of IgG1-DR5-01-G56T-E430G+IgG1-DR5-05-E430G inhibited tumor growth at 0.5 mg/kg, 2 mg/kg and 10 mg/kg in an in vivo SK-MES-1 human squamous cell lung carcinoma xenograft model.

### Example 11: Mouse PDX clinical trials

Patient-derived xenograft (PDX) models are generated by implantation and propagation of human tumor biopsy material in immunodeficient mice and are thought to represent the genetic and histological heterogeneity as observed in human tumors. A PDX clinical trial can be used to screen a large set of PDX models for drug sensitivity with the aim to predict the clinical trial response. We made use of PDX clinical trials, performed by Crown Bioscience at the Beijing, China facility and San Diego, US facility, to screen for sensitivity to the mixture of IgG1-DR5-01-G56T-E430G+IgG1-DR5-05-E430G in the following three solid tumor indications: CRC, NSCLC and kidney cancer.

For the models performed at Crown Bioscience, Beijing, China, fresh tumor tissues from mice bearing established primary human cancer tissues were harvested and cut into small pieces (approximately 2-3 mm in diameter). PDX tumor fragments, harvested from donor mice and washed with RPMI1640, were inoculated subcutaneously (s.c.) at the upper right dorsal flank of female BALB/c athymic Nude mice (Nanjing Biomedical Research Institute of Nanjing University, China) or NU/NU NUDE mice (Beijing Vital River Laboratory Animal Technology Co. Ltd, China) for tumor development.

For the models performed at CrownBio, San Diego, US, frozen cell suspensions obtained from PDX tumor fragments were thawed, washed in PBS, counted and resuspended in cold PBS at a concentration of 0.25×10⁶-1.0×10⁶ viable cells/mL. Cell suspensions were mixed with an equal volume of Cultrex ECM, withdrawn into a chilled 1 mL Luer-lok syringe fitted with a 26G 7/8 (0.5 mm×22 mm) needle and stored on ice. Immunodeficient 6-8 weeks old female NOD/SCID mice (Envigo and Jackson, US) were prepared for injection using standard isoflurane anesthesia and shaved prior to s.c. injection of 200 μL tumor cell suspension in ECM in the rear flank (range 0.5×10⁵−5×10⁵ cells/mouse).

Once tumors were palpable, tumor volumes were measured at least twice per week using calipers until tumor volume reached 1,500 mm³. Tumor volumes (mm³) were calculated as follows: tumor volume=0.5×(length)×(width)².

The PDX clinical trial was performed using a 1 mouse per group design: for each model, two mice with comparable tumor volume were enrolled in the study and treatments were started when the mean tumor volume reached ˜150-250 mm³. The mouse in the treatment group was treated once a week (QW) ×2 by i.v. injection of 10 μL 0.2 mg/mL antibody per gram body weight (2 mg/kg). The control mouse was treated with PBS QW×2.

Evaluation of response to treatment with the mixture of IgG1-DR5-01-G56T-E430G+IgG1-DR5-05-E430G was performed by comparing the change in tumor volume in mice treated with the antibody mixture (ΔT=tumor volume last day of analysis treated mouse−tumor volume day 0 treated mouse) with the change in tumor volume of PBS-treated control mice (AC=tumor volume last day of analysis control mouse−tumor volume day 0 control mouse). Response was evaluated between the day of randomization (initiation of treatment) and the last day of analysis (maximally 25 days after initiation of treatment, when exposure could reasonably be assumed). Models in which the tumor volume in the PBS-treated control mouse did not increase at least two-fold compared to the day treatment started were excluded from analysis. The relative tumor growth was calculated according to the formula ΔT/ΔC*100. Responding models were defined as models showing ΔT/ΔC<10% (tumor regression or tumor stasis), and non-responder models were defined as models showing ΔT/ΔC≥70%. The models that could not be classified as responder or non-responder (10%≤ΔT/ΔC<70%) were classified as intermediate. In total, 70 CRC, 62 NSCLC and 5 kidney cancer models were analyzed and categorized according to these criteria.

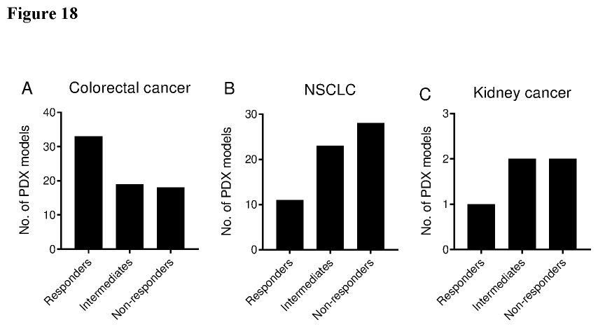

Of the CRC PDX models, 33/70 (47%) responded to the mixture of IgG1-DR5-01-G56T-E430G+IgG1-DR5-05-E430G, 19/70 (27%) were intermediates and 18/70 (26%) did not respond (FIG. 18A).

Of the NSCLC PDX models, 11/62 (18%) responded to the mixture of IgG1-DR5-01-G56T-E430G+IgG1-DR5-05-E430G, 23/62 (37%) were intermediates and 28/62 (45%) did not respond (FIG. 18B).

Of the kidney cancer PDX models, 1/5 (20%) responded to the mixture of IgG1-DR5-01-G56T-E430G+IgG1-DR5-05-E430G, 2/5 (40%) were intermediates and 2/5 (40%) did not respond (FIG. 18C).

### Example 12: Anti-tumor activity in colorectal cancer PDX models

Immunodeficient 8-13 weeks old female BALB/c athymic nude mice (Beijing HFK Bio-Technology Co. Ltd.) or nu/nu mice (Vital River Laboratories Research Models and Services) were inoculated subcutaneously at the right flank with one tumor fragment (2-3 mm diameter) at CrownBio, Beijing, China. Earmarks were placed for mouse identification. Tumor volumes were measured at least twice per week using a digital caliper (PLEXX). Tumor volumes (mm³) were calculated as follows: tumor volume=0.5×(length)×(width)². Mice were divided into groups of 8 mice each, with equal tumor size distribution (average and variance). Mice were treated QW×2 by i.v. injection of 10 μL IgG1-DR5-01-G56T-E430G+IgG1-DR5-05-E430G per gram body weight: 0.2 mg/mL (2 mg/kg) or 0.05 mg/mL (0.5 mg/kg).

### Colorectal Cancer PDX Model CR0126

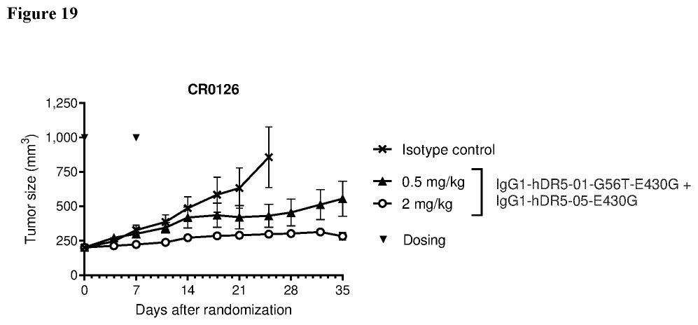

Tumor fragments of the colorectal cancer PDX model CR0126 were inoculated into the flank of 8-9 weeks old female nu/nu mice. Mice were assigned into groups using randomized block design and treatments were started at day 18 when the mean tumor size reached 201 mm³ (8 mice per group). Two mice died during the study, which was considered to be treatment unrelated, these mice were excluded from analysis. FIG. 19 shows mean tumor volumes per treatment group. Statistical analysis on the last day that all groups were intact (day 25 after start treatment) showed significant tumor growth inhibition by 2 mg/kg Hx-DR5-01/05 (Mann-Whitney test; p<0.0379) compared to the control IgG1-b12-E430G. No significant tumor growth inhibition was observed for 0.5 mg/kg Hx-DR5-01/05.

### Colorectal Cancer PDX Model CR3056

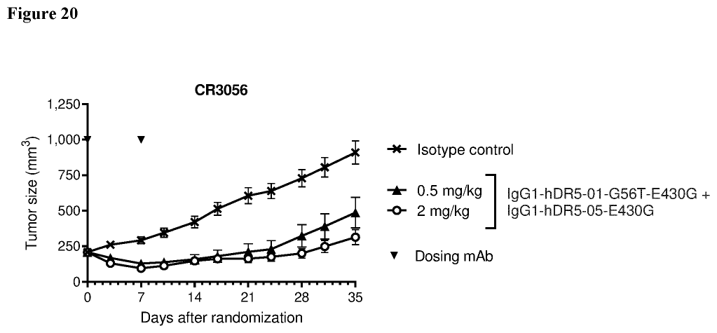

Tumor fragments of the colorectal cancer PDX model CR3056 was inoculated into the flank of 12-13 weeks old female BALB/c nude mice. Mice were assigned into groups using randomized block design and treatments were started at day 37 when the mean tumor size reached 208 mm³ (8 mice per group). FIG. 20 shows mean tumor volumes per treatment group. Statistical analysis on the last day that all groups were intact (day 42 after start treatment) showed significant tumor growth inhibition by both 2 mg/kg and 0.5 mg/kg Hx-DR5-01/05 (Mann-Whitney test; p<0.0003 and p<0.0379 respectively) compared to the control IgG1-b12-E430G.

### Colorectal Cancer PDX Model CR3150

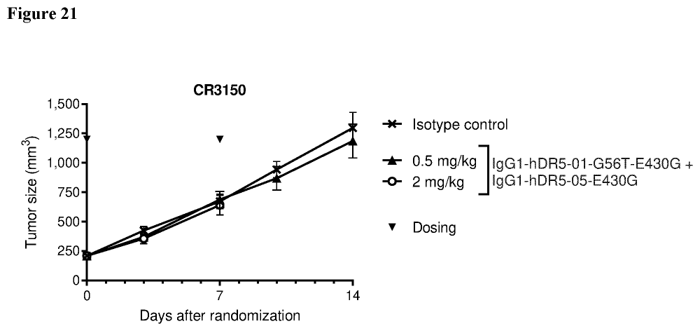

Tumor fragments of the colorectal cancer PDX model CR3056 was inoculated into the flank of 9-10 weeks old female BALB/c nude mice. Mice were assigned into groups using randomized block design and treatments were started at day 19 when the mean tumor size reached 208 mm³ (8 mice per group). FIG. 21 shows mean tumor volumes per treatment group. Statistical analysis on the last day that all groups were intact (day 7) shows that none of the treatments induced significant tumor growth inhibition.

### Example 13: PK in Tumor-Free Mice

The in vivo PK was explored in 7-10 weeks old female tumor-free immunodeficient CB17-SCID mice (C.B-17/IcrHan®Hsd-Prkdcscid, Harlan). Earmarks were placed for mouse identification.

Mice were intravenously injected with 0.2 mL of 0.1 mg/mL IgG1-DR5-01-G56T-E430G or IgG1-DR5-05-E430G, or the mixture thereof per mouse (20 μg/mouse, which equals around 1 mg/kg) with 3 mice/group. 50-100 μL blood samples were collected from the saphenous vein at 10 minutes, 4 hours, 1 day, 2 days, 7 days, 13 days and 20 days after antibody administration.

Blood was collected into heparin containing vials (Sarstedt, Cat no 20.1309), centrifuged for 5 minutes at 10,000×g and supernatant (plasma) was transferred to Eppendorf tubes. For human IgG analysis, plasma samples were diluted 1:20 for the first five time points (15 μL sample in 285 μL PBSTA [PBS; B.Braun; Cat no 3623140, with 0.05% Tween-20; Sigma-Aldrich; Cat no 63158, and 0.2% Bovine Serum Albumin; BSA; Roche; Cat no 10735086001]) and 1:10 for the last two time points (30 μL sample in 270 μL PBSTA) and stored at −20° C. until determination of antibody concentrations.

96-well flat bottom ELISA plates (Greiner bio-one; Cat no 655092) were coated overnight at 4° C. with 2 μg/mL mouse-anti-human IgG antibody, clone MH16-1 (Sanquin, Cat no M1268) in 100 μL PBS. After washing the plates three times in PBST (PBS with 0.05% Tween-20 [Sigma-Aldrich]), non-specific binding was blocked by adding 200 μL/well PBSA (PBS with 0.2% BSA [Roche]) and incubated for 1 hour at room temperature (RT). The wells were washed three times with PBST. Next, 100 μL diluted plasma samples (four serial dilutions for each sample in PBSTA; 100×, 300×, 900×, 2700× for the first five time points; 50×, 150×, 450×, 1350× for the last two time points) were added and incubated for 1 hour at RT while shaking (300 rpm). After washing three times with PBST, wells were incubated with 100 μL peroxidase-conjugated AffiniPure goat anti-human IgG Fey-specific antibody (Jackson; Cat no 109-035-098) 1:10,000 in PBSTA for one hour at RT while shaking (300 rpm). After washing three times with PBST, the reaction was visualized through an incubation with 100 μL 2,2′-azino-bis (3-ethylbenzothiazoline-6-sulfonic acid (ABTS [Roche; Cat no 11112597001]) for around 10 minutes at RT protected from light. The substrate reaction was stopped by adding an equal volume of 2% oxalic acid. Absorbance was measured at 405 nm on an ELISA reader (BioTek ELx808 Absorbance Microplate Reader). The injected material (range 0.037-1 μg/mL in 3-fold dilutions in PBSTA) was used to generate a standard curve, from which a calibration curve was calculated by interpolation of unknowns using a 4-parameter logistic fit curve in Microsoft Excel. Human IgG concentrations in the plasma samples were calculated from the equation of the calibration curve and plotted using GraphPad Prism software. As a plate control, purified human IgG1 (Binding Site, Cat no BP078) was included.
1. Area under the curve (AUC) from time point 0 to last measured time point was calculated using Graphpad Prism software.
2. Maximum observed plasma drug concentration (Cmax) is the maximal human IgG plasma concentration after administration of the antibody injection as measured 10 minutes after antibody injection.
3. Clearance rate (CL) until the last day of blood sampling (day 20 after antibody injection) was determined by the formula: (Dose×1,000)/AUC.
4. Central volume of distribution (Vcen) was determined by the formula: (Dose x 1000)/Cmax.

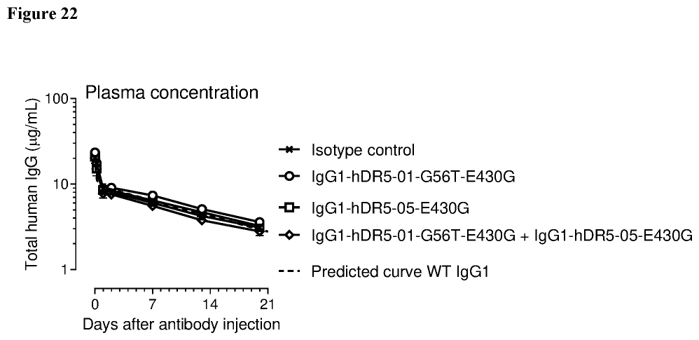

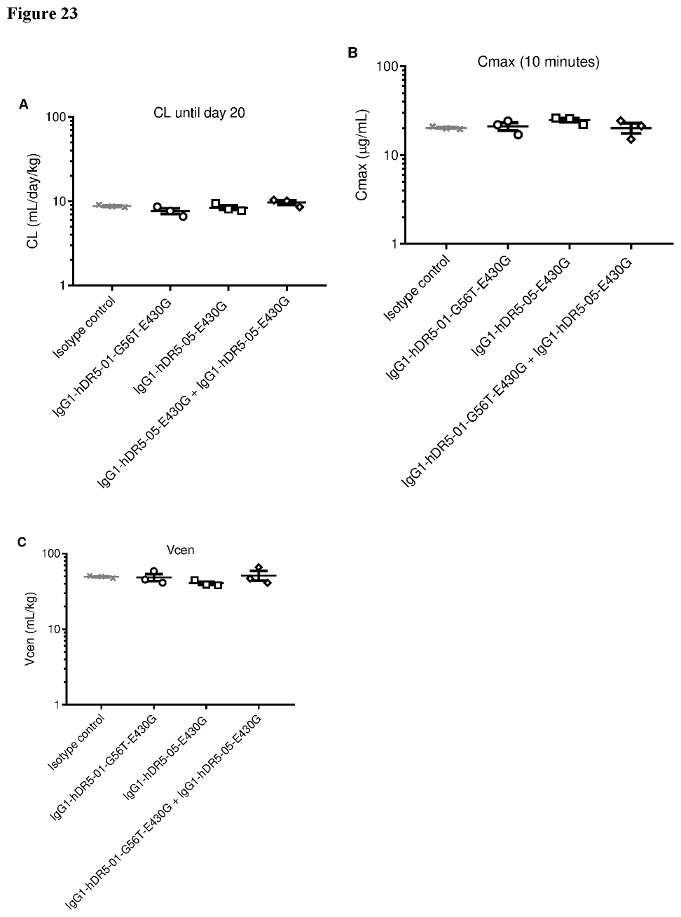

FIG. 22 shows the total human plasma IgG concentration in time. The predicted IgG1 curve was based on a two-compartment model. Curves of the antibody test samples did not deviate from the predicted curve of a non-binding human IgG1 in mice. No differences in the CL, Cmax and Vcen were observed between IgG1-hDR5-01-G56T-E430G, IgG1-hDR5-05-E430G and the isotype control antibody (FIG. 23). The presented data demonstrate that the observed pharmacokinetic properties of IgG1-DR5-01-G56T-E430G, IgG1-DR5-05-E430G, and the mixture thereof were comparable to normal human IgG1 in tumor-free mice.

### Example 14: PK in Cynomolgus Monkey

### Single Dose Study of the Mixture of IgG1-hDR5-05-E430G+IgG1-hDR5-01-G56T-E430G by Intravenous Infusion in Cynomolgus Monkeys

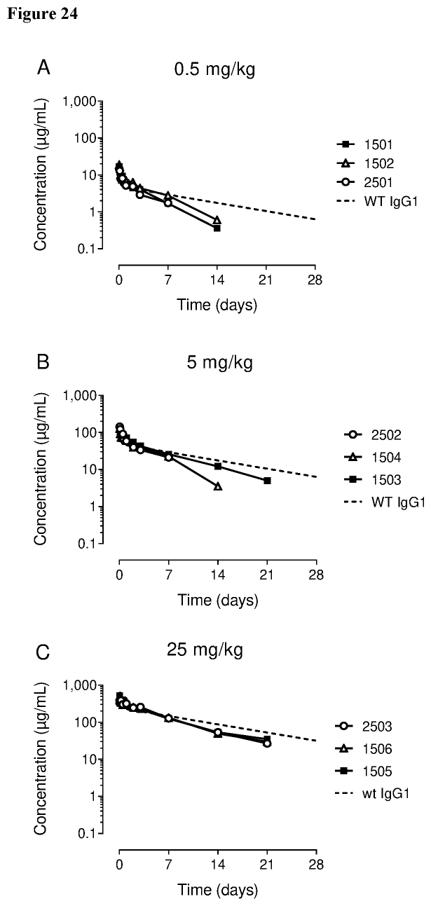

Human antibody plasma concentration profiles were measured using a generic IgG PK electrochemiluminescence immunoassay (ECLIA) after intravenous single dose infusion of the mixture of IgG1-hDR5-05-E430G+IgG1-hDR5-01-G56T-E430G at dose levels of 0.5, 5 and 25 mg/kg (infusion time 30 min, n=3 females, time points post-dose: 1, 3, 6, 12, 24 hours, 2, 3, 7, 14 and 21 days). In short, IgG1-hDR5-05-E430G+IgG1-hDR5-01-G56T-E430G were captured on a coat of monoclonal anti Human IgG (non-cross reactive with Cynomolgus IgG) antibody. Captured IgG was detected by using another monoclonal anti Human IgG (non-cross reactive with Cynomolgus IgG) conjugated to SULFO-TAG. This complex was visualized using an ECL imager. Plasma concentration-time profiles were consistent with the intravenous dose route of the test item (FIG. 24). Individual estimates of the half-life (T_{1/2}) ranged from 3.16 to 7.57 days. Individual clearance values ranged from 8.98 to 14.2 mL/day/kg. Individual volumes of distribution values ranged from 52.4 to 98.8 mL/kg, indicating a limited distribution of the test item beyond the circulation. Based on nAUC_{0-∞}, a more or less proportional increase in exposure was seen with increasing dose. Based on nCₘₐₓ, a proportional increase in exposure was seen up to a dose of 5 mg/kg, whereas between 5 and 25 mg/kg a less than proportional increase in exposure was observed. Individual plasma concentration profiles are shown in FIG. 24 and group mean toxicokinetic parameters are shown in Table 13.

TABLE 13
Mean pharmacokinetic parameters in single i.v. dose study of IgG1-hDR5-05-E430G +
IgG1-hDR5-01-G56T-E430G in cynomolgus monkeys

| | | | | | | nAUC_{0-∞} | | |
|---|---|---|---|---|---|---|---|---|
| | | | nCₘₐₓ | | | (μg · | | |
| | | | (μg · | | AUC_{0-∞} | day · | CL | |
| Dose | | Cₘₐₓ | mL⁻¹/ | | (μg · | mL⁻¹/ | (mL | Vd |
| (mg · | Tₘₐₓ | (μg · | mg · | T_{1/2} | day/ | mg · | day⁻¹/ | (mL · |
| kg⁻¹) | (day) | mL⁻¹) | kg⁻¹) | (day) | ·mL⁻¹) | kg⁻¹) | ·kg⁻¹) | kg⁻¹) |
| 0.5 | 0.0417 | 16.7 | 33.5 | 3.48 | 40.3 | 80.6 | 12.6 | 63.5 |
| 5 | 0.0417 | 135.0 | 27.0 | 4.59 | 452.6 | 90.4 | 11.4 | 72.6 |
| 25 | 0.14 | 470.3 | 18.8 | 6.93 | 2748 | 109.9 | 9.10 | 91.0 |

### Dose Range Finding Study of the Mixture of IgG1-hDR5-05-E430G+IgG1-hDR5-01-G56T-E430G by Intravenous Infusion in Cynomolgus Monkeys

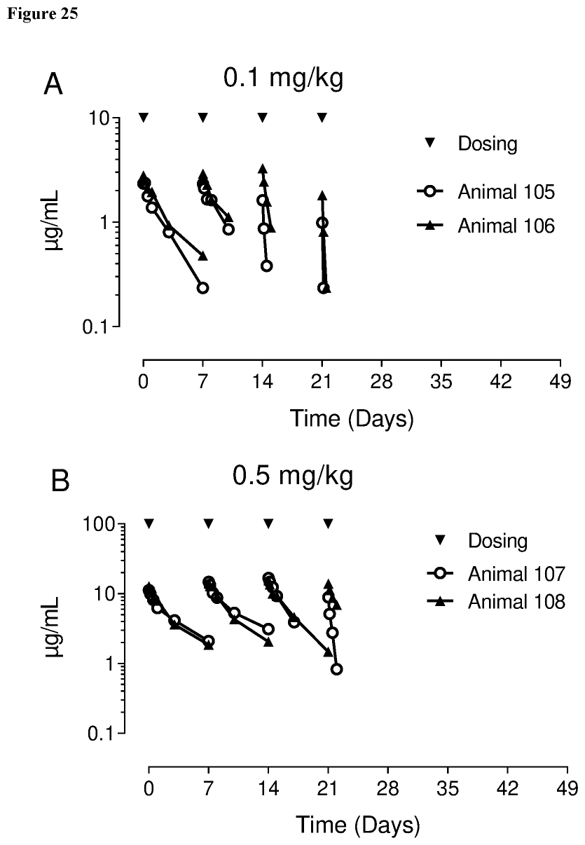

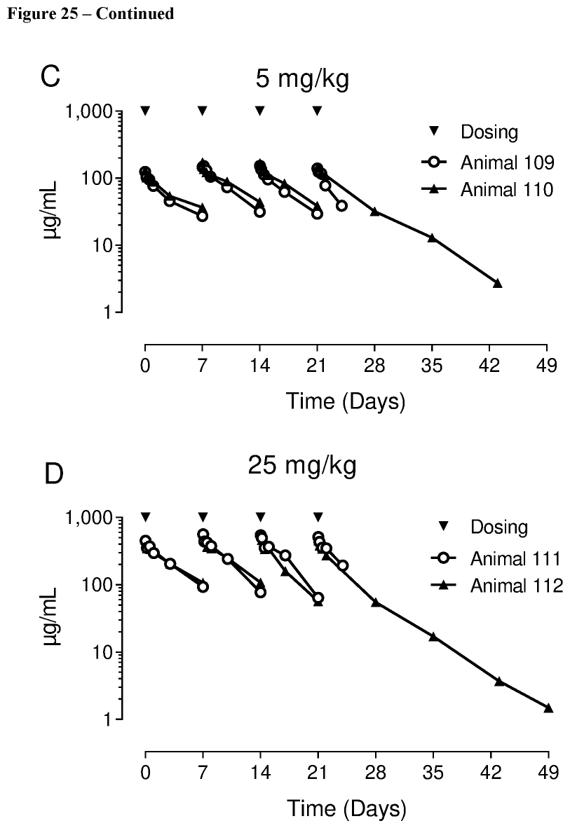

Human antibody plasma concentration profiles were measured using a generic IgG PK ECLIA after i.v. single dose (n=1) or repeated dose (1q4×4, n=2) infusion of the mixture of IgG1-hDR5-05-E430G+IgG1-hDR5-01-G56T-E430G at dose levels of 0.1, 0.5, 5 and 25 mg/kg (infusion time 30 min, sampling time points pre-dose and 0.5, 4, 12, 24 and 72 hours post-dose). Plasma concentration-time profiles were consistent with a wild type human IgG1 clearance in cynomolgus monkeys, with no indications of target-mediated clearance (FIG. 25). Based on nCₘₐₓ and nAUC_{0-∞}, a proportional increase in exposure was seen with increasing dose; based on nCₘₐₓ a less than proportional increase in exposure was observed between the mid- and high-dose groups, only in the single-dose treated groups.

Repeat-Dose Toxicity Study with 5 Repeated Weekly i.v. Infusions of the Mixture of IgG1-hDR5-05-E430G+IgG1-hDR5-01-G56T-E430G in Cynomolgus Monkeys

Four groups of five male and five female cynomolgus monkeys each received once-weekly 30 minute intravenous infusions, on Days 1, 8, 15, 22 and 29 of the dosing phase, as follows:

| Group | Group | Dose level |
|---|---|---|
| number | description | (mg/kg) |
| 1 | Control | 0 (vehicle) |
| 2 | Low | 2 |
| 3 | Intermediate | 10 |
| 4 | High | 50 |

For Days 1, 8, 15, 22 and 29, blood samples were taken from each animal pre-dose and 0.5, 4, 12, 24 and 72 hours after the end of infusion. Additional blood samples were taken from each recovery animal (two males and two females per dose group) on Days 36, 43, 50 and 57.

Concentrations of IgG1-hDR5-01-G56T-E430G in cynomolgus monkey plasma were determined using an ECLIA method (based on Example 5). In this assay of IgG1-hDR5-01-G56T-E430G is captured with coating antigen DR5sh79-115ECDdelHis (SEQ ID NO 43). Captured of IgG1-hDR5-01-G56T-E430G was detected by a monoclonal anti Human IgG (non-cross reactive with Cynomolgus IgG) antibody conjugated to SULFO-TAG. The complex was visualized using an ECL imager. This ECLIA detects only of IgG1-hDR5-01-G56T-E430G, not IgG1-hDR5-05-E430G.

Concentrations of IgG1-hDR5-05-E430G in cynomolgus monkey plasma were determined using an ECLIA method (based on Example 5). In this assay, IgG1-hDR5-05-E430G is captured with coating antigen DR5sh139-166ECDdelHis (SEQ ID NO 44). Captured IgG1-hDR5-05-E430G was detected by a monoclonal anti Human IgG (non-cross reactive with Cynomolgus IgG) antibody conjugated to SULFO-TAG. The complex was visualized using an ECL imager. This ECLIA detects only IgG1-hDR5-05-E430G, not IgG1-hDR5-01-G56T-E430G.

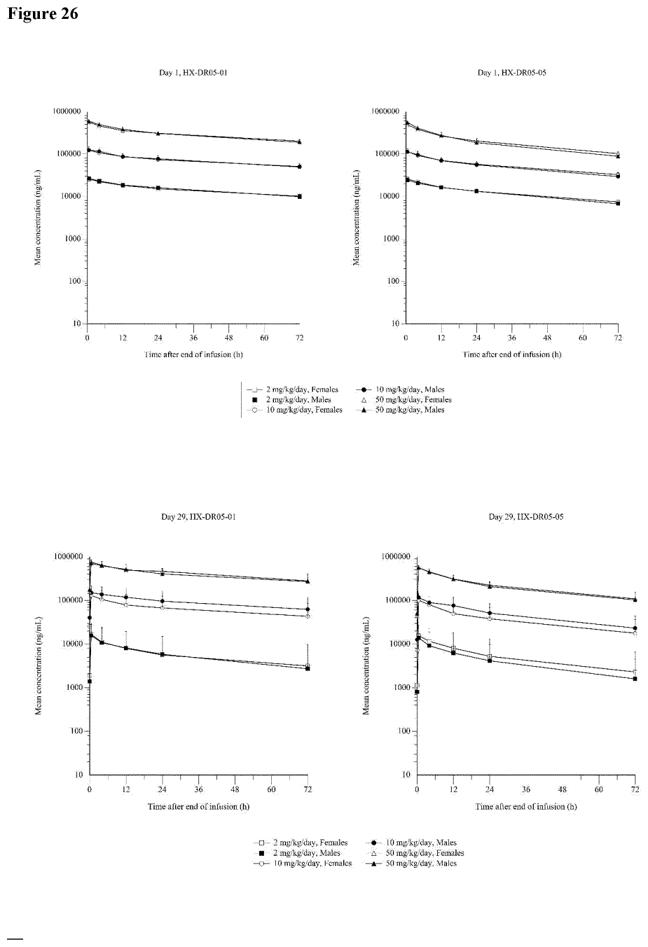

FIG. 26 shows the mean plasma concentration of IgG1-hDR5-01-G56T-E430G and IgG1-hDR5-05-E430G on dosing days 1 and 29. On each sampling occasion, plasma concentrations of both IgG1-hDR5-01-G56T-E430G and IgG1-hDR5-05-E430G were generally at a maximum at the first sampling time point, 1 hour after the start of infusion (0.5 hours after the end of infusion).

Clearance of both IgG1-hDR5-01-G56T-E430G and IgG1-hDR5-05-E430G from the systemic circulation was incomplete within the dosing interval after each of the first two once-weekly doses. The extent of clearance of both IgG1-hDR5-01-G56T-E430G and IgG1-hDR5-05-E430G increased following subsequent weekly doses, particularly at the 2 mg/kg/week dose level. On Day 1, where calculable, total serum clearance was between 0.649 and 1.23 mL/h/kg for IgG1-hDR5-01-G56T-E430G and between 0.936 and 1.65 mL/h/kg for IgG1-hDR5-05-E430G. On subsequent sampling occasions, mean Cl₅₅ values tended to increase with time. The most rapid clearance tended to occur in the animals that were anti-drug antibody (ADA)-positive and gave the highest ADA responses.

On Day 1, where calculable, elimination half-lives (t_{1/2}) were between 83.1 and 103 hours for IgG1-hDR5-01-G56T-E430G and between 54.5 and 95.4 hours for IgG1-hDR5-05-E430G. On Day 8, where calculable, t_{1/2} was between 26.4 and 103 hours for IgG1-hDR5-01-G56T-E430G and between 27.3 and 89.1 hours for IgG1-hDR5-05-E430G. After subsequent weekly doses, the mean half-life for both compounds tended to decrease, particularly at the low and intermediate dose levels, with greater inter-animal variability.

The observed increases in measures of plasma exposure (Cₘₐₓ and AUC₍₀₋ₜ₎) to both IgG1-hDR5-01-G56T-E430G and IgG1-hDR5-05-E430G following once-weekly intravenous administration were approximately proportional to the increases in dose level. These data suggest that there was no saturation of the clearance of either compound at the higher dose levels.

### Example 15: Human Equivalent Dose

For safety reasons, a lower FIH starting dose than the no observed adverse events level (NOAEL)/highest non severely toxic dose (HNSTD)-based maximum recommended starting dose (MRSD) of the mixture of IgG1-hDR5-01-G56T-E430G+IgG1-hDR5-05-E430G of 8.3 mg/kg was used. A FIH clinical trial starting dose of the mixture of IgG1-hDR5-01-G56T-E430G+IgG1-hDR5-05-E430G of 0.3 mg/kg was used. This dose level was considered to be safe and in the lower end of the potential therapeutically active dose range, based on considerations from nonclinical pharmacology, pharmacokinetic and toxicology studies and a preclinical population PK simulation model (performed by BAST GmbH). For modelling purposes, only cynomolgus monkey PK data from the first dosing cycles were used to avoid confounding effects of anti-drug antibodies that were observed upon subsequent dosings.
1. The NOAEL and HNSTD in cynomolgus monkey following a 1QW×5 i.v. dosing of the mixture of IgG1-hDR5-01-G56T-E430G+IgG1-hDR5-05-E430G was determined in the pivotal good laboratory practice (GLP) i.v. toxicity study to be 50 mg/kg, which was converted to an MRSD of 8.3 mg/kg in human.

| | | MRSDₕᵤₘₐₙ |
|---|---|---|
| NOAEL_{cyno} | HNSTD_{cyno} | (=NOAEL_{cyno}/6) |
| (mg/kg) | (mg/kg) | (mg/kg) |
| 50 | 50 | 8.3 |

The in vivo pharmacologically active dose of the mixture of IgG1-hDR5-01-G56T-E430G+IgG1-hDR5-05-E430G as determined using CDX (described in Example 10) and PDX mouse models (described in Example 11) was used for conversion to a human equivalent dose level using a preclinical population PK simulation model (performed by BAST GmbH). Based on these predictions, a human dose of 0.3 mg/kg of the mixture of IgG1-hDR5-01-G56T-E430G+IgG1-hDR5-05-E430G is considered to correspond to the in vivo murine dose level range of 0.5 mg/kg that induced a partial anti-tumor response in the mouse xenograft models. Therefore, the FIH starting dose of 0.3 mg/kg was considered to be in the lower end of a potential therapeutic dose-range in human patients.

| | In vivo | | |
|---|---|---|---|
| | anti-tumor | | |
| | response in | Predicted human | |
| | CDX and PDX | equivalent dose | |
| | mouse models | (mg/kg) | |
| | (mg/kg) | AUC-based | Cₘₐₓ-based |
| Partial response | 0.5 | 0.2 | 0.32 |
| Full response | 2 | 0.8 | 1.26 |

IC20 values of the in vitro cytotoxicity of the mixture of IgG1-hDR5-01-G56T-E430G+IgG1-hDR5-05-E430G in various human cancer cell lines were used for conversion to the minimal anticipated biological effect level (MABEL) in humans. From the data described in Example 7, the average IC20 values for the cell lines for which more than 40% inhibition of cell viability was observed with the mixture of IgG1-hDR5-01-G56T-E430G+IgG1-hDR5-05-E430G were used to calculate the median IC20 value to be 0.554 nM (0.083 μg/mL) (Table 14). Conversion to a corresponding human dose level was performed using the preclinical population PK simulation model (performed by BAST GmbH), resulted in a MABEL dose of 0.0051 mg/kg in human patients.

TABLE 14
Average IC20 and percentage of maximal growth inhibition
from a three-day viability assay with IgG1-hDR5-01-G56T-
E430G + IgG1-hDR5-05-E430G performed with the cell
lines used for MABEL, i.e. showing >40 max inhibition.

| | | | | Max |
|---|---|---|---|---|
| | Cancer | Average IC20 | | inhibition |
| Cell line | subtype | nM | μg/mL | % |
| A375 | Melanoma | 0.445 | 0.067 | 54.6 |
| BxPC-3 | Pancreatic | 0.677 | 0.102 | 89.4 |
| PANC-1 | Pancreatic | 0.554 | 0.083 | 61.0 |
| COLO 205 | CRC | 0.103 | 0.015 | 99.6 |
| HCT15 | CRC | 0.692 | 0.104 | 66.7 |
| SK-MES-1 | NSCLC | 0.572 | 0.086 | 76.6 |
| SNU-5 | Gastric | 0.274 | 0.041 | 44.7 |

| | | |
|---|---|---|
| Overall Median | 0.554 | 0.083 |

The MABEL-based starting dose of 0.0051 mg/kg derived from in vitro cytotoxicity studies with GEN1029 was not considered appropriate for treatment of patients with advanced cancer for the following reasons: 1) the mixture of IgG1-hDR5-01-G56T-E430G+IgG1-hDR5-05-E430G has shown no immune agonistic properties, and 2) no hazard of acute cytokine-releasing activity has been identified with the mixture of IgG1-hDR5-01-G56T-E430G+IgG1-hDR5-05-E430G.

The nonclinical population PK model was also used to simulate the potential plasma concentration of IgG1-hDR5-01-G56T-E430G and IgG1-hDR5-05-E430G after repeated 2-weekly (1Q2W) i.v. treatment of humans at an assumed therapeutically active dose level of 1 mg/kg of the mixture. According to the in vitro pharmacology studies, the predicted plasma concentrations of IgG1-hDR5-01-G56T-E430G and IgG1-hDR5-05-E430G at trough time after a dose of 1 mg/kg of the mixture was considered to be therapeutically active.

### Example 16: PK in humans

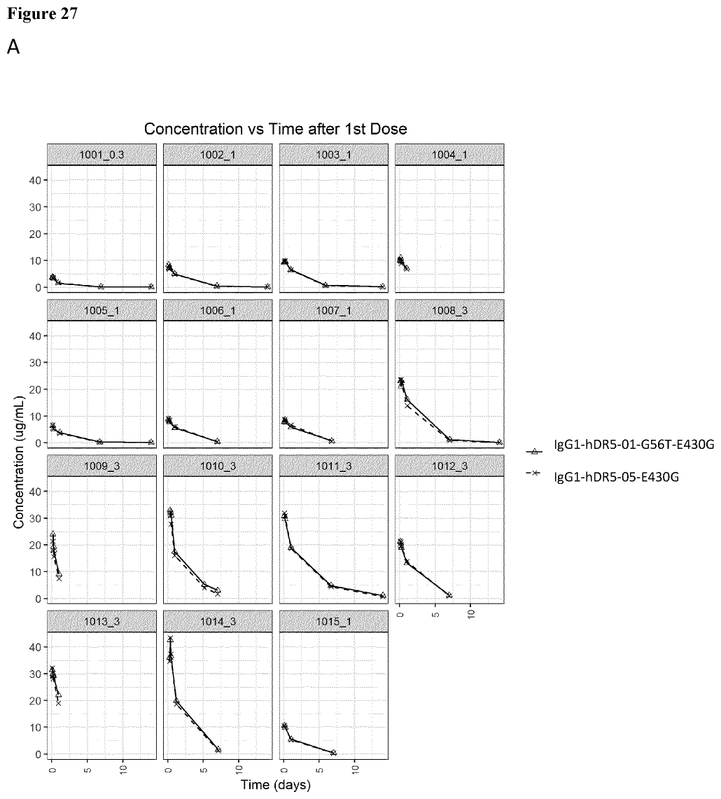

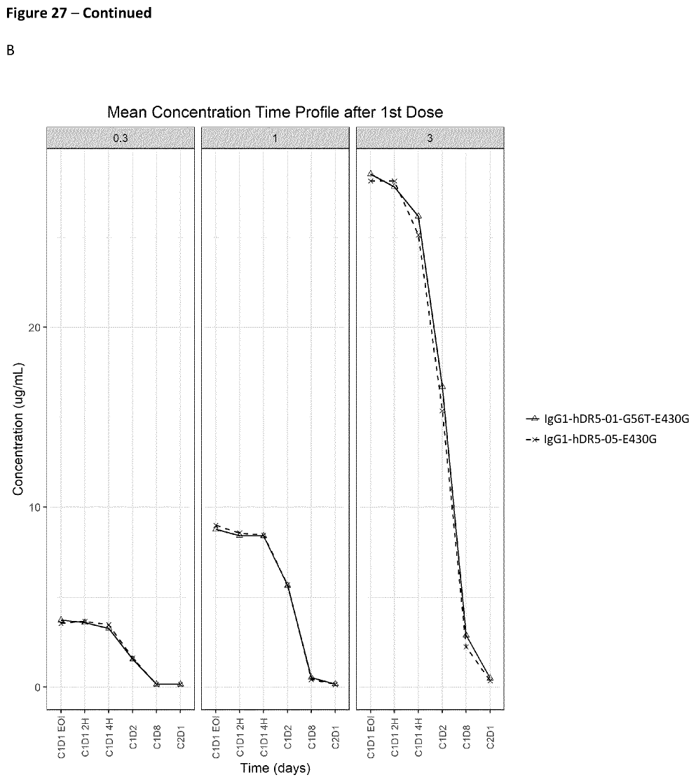

Available PK data from the FIH clinical trial GCT1029-01 with the mixture of IgG1-hDR5-01-G56T-E430G+IgG1-hDR5-05-E430G evaluated after the first dose of 0.3, 1 or 3.0 mg/kg in dose escalation cohorts shows that the PK in human appears to be very similar for the two molecules IgG1-hDR5-01-G56T-E430G and IgG1-hDR5-05-E430G (FIG. 27). In addition, plasma half-life (T_{1/2}) was found to be relatively short ranging from 15 to 80 hours. These data indicate that the human PK predictions based on the preclinical PK model as described in Example 15 have underestimated the human clearances of both IgG1-hDR5-01-G56T-E430G and IgG1-hDR5-05-E430G, thereby resulting in C_{trough} values of both antibodies below the lower limit of quantification (LLOQ) (based on bi-weekly dosing interval) and AUC_{0-14 days} below the predicted values. Therefore, the applied bi-weekly dosing regimen may not allow to achieve the best therapeutic index, and an intensified weekly schedule and priming doses have been postulated to be able to increase the dose and thereby obtain higher drug exposures and likely improved anti-tumor efficacy.

1. A method of treating a solid tumor or a hematological malignancy in a subject, the method comprising administering to a subject in need thereof a first antibody that binds DR5 and a second antibody that binds DR5, or a pharmaceutically acceptable salt thereof, wherein the first antibody and the second antibody is administered on,
i) day 1 and day 8 of a 14-days cycle for the first four cycles; or
ii) day 1 and day 8 of a first 14-day cycle; or
iii) day 1 of a first 14-day cycle; or
iv) day 1 of a first and second 14-day cycle;
followed by administration on day 1 of a 14-day cycle.

2. The method of claim 1, wherein the first antibody comprises a variable heavy chain region and a variable light chain region wherein the variable heavy chain region comprises the CDR1, CDR2 and CDR3 sequences of SEQ ID Nos: 1, 8, and 3 respectively; and wherein the variable light chain region comprises the CDR1, CDR2 and CDR3 sequences of SEQ ID Nos: 5, FAS, and 6 respectively.

3. The method of claim 1, wherein the second antibody comprises a variable heavy chain region and a variable light chain region wherein the variable heavy chain region comprises the CDR1, CDR2 and CDR3 sequences of SEQ ID Nos: 10, 2, and 11 respectively; and wherein the variable light chain region comprises the CDR1, CDR2 and CDR3 sequences of SEQ ID Nos: 13, RTS, and 14 respectively.

4. The method of any one of claims 1-3, wherein the first and second antibody comprises an Fc region of a human IgG1, wherein the Fc region comprises an E430G mutation of an amino acid position corresponding E430 in human IgG1, wherein the amino acid position is according to the Eu numbering.

5. The method of any one of claims 1 to 4, wherein the first antibody comprises the heavy chain and light chain as set forth in SEQ ID NO 30 and 27, respectively.

6. The method of any one of claims 1 to 4, wherein the first antibody comprises the heavy chain and light chain as set forth in SEQ ID NO 47 and 27, respectively.

7. The method of any one of claims 1 to 6, wherein the second antibody comprises the heavy chain and light chain as set forth in SEQ ID NO 48 and 35, respectively

8. The method of any one of claims 1 to 6, wherein the second antibody comprises the heavy chain and light chain as set forth in SEQ ID NO 32 and 35, respectively.

9. The method of any one of claims 1-8, wherein the solid tumor is selected from the group consisting of: colorectal cancer (CRC), non-small lung cancer (NSCLC), triple negative breast cancer (TNBC), renal cell carcinoma (RCC), gastric cancer, pancreatic cancer and urothelial cancer.

10. The method of any one of claims 1-8, wherein the hematological malignancy is selected from the group consisting of: leukemia, myeloid leukemia, acute myeloid leukemia, myelodysplastic syndrome, lymphoma, Non-Hodgkin lymphoma, Hodgkin Lymphoma, diffuse large B-cell lymphoma, mantle cell lymphoma, multiple myeloma, chronic lymphocytic leukemia or myelodysplastic syndromes.

11. The method of any one of the preceding claims, wherein the first and second antibody, or a pharmaceutically acceptable salt thereof, is administered simultaneously, separately, or sequentially.

12. The method of any one of the preceding claims, wherein the first and second antibody, or a pharmaceutically acceptable salt thereof, is administered simultaneously.

13. The method of any one of the preceding claims, wherein the first and second antibody, or a pharmaceutically acceptable salt thereof, is administered by intravenous infusion.

14. The method according to any one the preceding claims, wherein the first or second antibody, or a pharmaceutically acceptable salt thereof, is administered at a dose ranging from about 0.05 mg/kg to 9 mg/kg.

15. The method according to any one the preceding claims, wherein the first or second antibody, or a pharmaceutically acceptable salt thereof, is administered at a dose of about 0.05 mg/kg, 0.15 mg/kg 0.3 mg/kg, 0.5 mg/kg, 1 mg/kg, 1.5 mg/kg, 2.25 mg/kg, 3 mg/kg, 4.5 mg/kg, 6 mg/kg, 7.5 mg/kg or 9 mg/kg.

16. The method according to any one the preceding claims, wherein the first or second antibody, or a pharmaceutically acceptable salt thereof, is administered to the subject on day 1 and day 8 of a 14-days cycle for the first four cycles at a dose ranging from about 0.15 mg/kg to 9 mg/kg.

17. The method according to any one of claims 1 to 15, wherein the first or second antibody, or a pharmaceutically acceptable salt thereof is administered to the subject on day 1 and day 8 of a first 14-day cycle at a dose ranging from about 0.15 mg/kg to 3 mg/kg.

18. The method according to any one of claims 1 to 15, wherein the first or second antibody, or a pharmaceutically acceptable salt thereof is administered to the subject on day 1 of a first 14-day cycle at a dose ranging from about 0.05 mg/kg to 1 mg/kg, such as ranging from about 0.05 mg/kg to 0.3 mg/kg.

19. The method according to any one of claims 1 to 15, wherein the first or second antibody, or a pharmaceutically acceptable salt thereof is administered to the subject on day 1 of a first and second 14-day cycle at a dose ranging from about 0.05 mg/kg to 1 mg/kg, such as ranging from about 0.05 mg/kg to 0.3 mg/kg.

20. The method according to any one of claims 1 to 15, wherein the first or second antibody, or a pharmaceutically acceptable salt thereof is administered to the subject on day 8 of a first 14-day cycle at a dose ranging from about 0.5 mg/kg to 3 mg/kg.

21. The method according to any one the preceding claims, wherein when the first and second antibody, or a pharmaceutically acceptable salt thereof, are combined then the total amount of antibody administered is at a dose ranging from about 0.1 mg/kg to 18 mg/kg.

22. The method according to any one of the proceeding claims, wherein the first and second antibody, or a pharmaceutically acceptable salt thereof, is administered at about a 1:1 molar ratio.

23. The method of any one of the preceding claims, wherein prior to administration of the first and second antibody a steroid hormone is administered.

24. The method according to claim 23, wherein the steroid hormone is a corticosteroid.

25. The method according to claims 23 to 24, wherein the steroid hormone is dexamethasone.

26. The method according to claims 23 to 25, wherein the dexamethasone is administered at a dose ranging from 1 to 100 mg.

27. The method according to claims 23 to 26, wherein the dexamethasone is administered at a dose of 10 mg.

28. The method according to claims 20 to 27, wherein the dexamethasone is administered by intravenous infusion.

29. A first and second antibody that binds DR5, or a pharmaceutically acceptable salt thereof, for use in the treatment of a solid tumor or a hematological malignancy, wherein the first antibody and the second antibody, or pharmaceutically acceptable salt thereof, is administered on,
i) day 1 and day 8 of a 14-days cycle for the first four cycles; or
ii) day 1 and day 8 of a first 14-day cycle; or
iii) day 1 of a first 14-day cycle, or
iv) day 1 of a first and second 14-day cycle;
followed by administration on day 1 of a 14-day cycle.

30. The first and second antibody that binds DR5, or a pharmaceutically acceptable salt thereof, for use according to claim 29, wherein the first antibody comprises a variable heavy chain region and a variable light chain region wherein the variable heavy chain region comprises the CDR1, CDR2 and CDR3 sequences of SEQ ID Nos: 1, 8, and 3 respectively; and wherein the variable light chain region comprises the CDR1, CDR2 and CDR3 sequences of SEQ ID Nos: 5, FAS, and 6 respectively.

31. The first and second antibody that binds DR5, or a pharmaceutically acceptable salt thereof, for use according to any one of claims 29 to 30, wherein the second antibody comprises a variable heavy chain region and a variable light chain region wherein the variable heavy chain region comprises the CDR1, CDR2 and CDR3 sequences of SEQ ID Nos: 10, 2, and 11 respectively; and wherein the variable light chain region comprises the CDR1, CDR2 and CDR3 sequences of SEQ ID Nos: 13, RTS, and 14 respectively.

32. The first and second antibody that binds DR5, or a pharmaceutically acceptable salt thereof, for use according to any one of claims 29 to 31, wherein the first and second antibody comprises an Fc region of a human IgG1, wherein the Fc region comprises an E430G mutation of an amino acid position corresponding E430 in human IgG1, wherein the amino acid position is according to the Eu numbering.

33. The first and second antibody that binds DR5, or a pharmaceutically acceptable salt thereof, for use according to any one of claims 29 to 32, wherein the first antibody comprises the heavy chain and light chain as set forth in SEQ ID NO 30 and 27, respectively.

34. The first and second antibody that binds DR5, or a pharmaceutically acceptable salt thereof, for use according to any one of claims 29 to 32, wherein the first antibody comprises the heavy chain and light chain as set forth in SEQ ID NO 47 and 27, respectively.

35. The first and second antibody that binds DR5, or a pharmaceutically acceptable salt thereof, for use according to any one of claims 29 to 34, wherein the second antibody comprises the heavy chain and light chain as set forth in SEQ ID NO 31 and 35, respectively.

36. The first and second antibody that binds DR5, or a pharmaceutically acceptable salt thereof, for use according to any one of claims 29 to 34, wherein the second antibody comprises the heavy chain and light chain as set forth in SEQ ID NO 48 and 35, respectively.

37. The first and second antibody that binds DR5, or a pharmaceutically acceptable salt thereof, for use according to any one of claims 29 to 36, wherein the solid tumor is selected from the group consisting of: colorectal cancer (CRC), non-small lung cancer (NSCLC), triple negative breast cancer (TNBC), renal cell carcinoma (RCC), gastric cancer, pancreatic cancer and urothelial cancer.

38. The first and second antibody that binds DR5, or a pharmaceutically acceptable salt thereof, for use according to any one of claims 29 to 36, wherein the hematological malignancy is selected from the group consisting of: leukemia, including chronic lymphocytic leukemia and myeloid leukemia, including acute myeloid leukemia and chronic myeloid leukemia, lymphoma, Non-Hodgkin lymphoma or multiple myeloma, Hodgkin Lymphoma or myelodysplastic syndromes.

39. The first and second antibody that binds DR5, or a pharmaceutically acceptable salt thereof, for use according to any one of claims 29 to 38, wherein the first and second antibody, or a pharmaceutically acceptable salt thereof, is administered simultaneously, separately, or sequentially.

40. The first and second antibody that binds DR5, or a pharmaceutically acceptable salt thereof, for use according to any one of claims 29 to 38, wherein the first and second antibody, or a pharmaceutically acceptable salt thereof, is administered simultaneously.

41. The first and second antibody that binds DR5, or a pharmaceutically acceptable salt thereof, for use according to any one of claims 29 to 40, wherein the first and second antibody, or a pharmaceutically acceptable salt thereof, is administered by intravenous infusion.

42. The first and second antibody that binds DR5, or a pharmaceutically acceptable salt thereof, for use according to any one of claims 29 to 41, wherein the first or second antibody, or a pharmaceutically acceptable salt thereof, is administered at a dose ranging from about 0.05 mg/kg to 9 mg/kg.

43. The first and second antibody that binds DR5, or a pharmaceutically acceptable salt thereof, for use according to any one of claims 29 to 42, wherein the first or second antibody, or a pharmaceutically acceptable salt thereof, is administered at a dose of about 0.05 mg/kg, 0.15 mg/kg, 0.3 mg/kg, 0.5 mg/kg, 1 mg/kg, 1.5 mg/kg, 2.25 mg/kg, 3 mg/kg, 4.5 mg/kg, 6 mg/kg, 7.5 mg/kg or 9 mg/kg.

44. The first and second antibody that binds DR5, or a pharmaceutically acceptable salt thereof, for use according to any one of claims 29 to 43, wherein the first or second antibody, or a pharmaceutically acceptable salt thereof, is administered to the subject on day 1 and day 8 of a 14-days cycle for the first four cycles at a dose ranging from about 0.15 mg/kg to 9 mg/kg.

45. The first and second antibody that binds DR5, or a pharmaceutically acceptable salt thereof, for use according to any one of claims 29 to 43, wherein the first or second antibody, or a pharmaceutically acceptable salt thereof is administered to the subject on day 1 and day 8 of a first 14-day cycle at a dose ranging from about 0.15 mg/kg to 3 mg/kg.

46. The first and second antibody that binds DR5, or a pharmaceutically acceptable salt thereof, for use according to any one of claims 29 to 43, wherein the first or second antibody, or a pharmaceutically acceptable salt thereof is administered to the subject on day 1 of a first 14-day cycle at a dose ranging from about 0.05 mg/kg to 1 mg/kg, such as ranging from about 0.05 mg/kg to 0.3 mg/kg.

47. The first and second antibody that binds DR5, or a pharmaceutically acceptable salt thereof, for use according to any one of claims 29 to 43, wherein the first or second antibody, or a pharmaceutically acceptable salt thereof is administered to the subject on day 1 of a first and second 14-day cycle at a dose ranging from about 0.05 mg/kg to 1 mg/kg, such as ranging from about 0.05 mg/kg to 0.3 mg/kg.

48. The first and second antibody that binds DR5, or a pharmaceutically acceptable salt thereof, for use according to any one of claims 29 to 43, wherein the first or second antibody, or a pharmaceutically acceptable salt thereof is administered to the subject on day 8 of a first 14-day cycle at a dose ranging from about 0.5 mg/kg to 3 mg/kg.

49. The first and second antibody that binds DR5, or a pharmaceutically acceptable salt thereof, for use according to any one of claims 29 to 48, wherein when the first and second antibody, or a pharmaceutically acceptable salt thereof, are combined then the total amount of antibody administered is at a dose ranging from about 0.1 mg/kg to 18 mg/kg.

50. The first and second antibody that binds DR5, or a pharmaceutically acceptable salt thereof, for use according to any one of claims 29 to 49, wherein the first and second antibody, or a pharmaceutically acceptable salt thereof, is administered at about a 1:1 ratio.

51. The first and second antibody that binds DR5, or a pharmaceutically acceptable salt thereof, for use according to any one of claims 29 to 50, wherein prior to administration of the first and second antibody a steroid hormone is administered.

52. The first and second antibody that binds DR5, or a pharmaceutically acceptable salt thereof, for use according to any one of claims 29 to 51, wherein the steroid hormone is a corticosteroid.

53. The first and second antibody that binds DR5, or a pharmaceutically acceptable salt thereof, for use according to any one of claims 29 to 52, wherein the steroid hormone is dexamethasone.

54. The first and second antibody that binds DR5, or a pharmaceutically acceptable salt thereof, for use according to any one of claims 29 to 53, wherein the steroid hormone is administered at a dose ranging from 5 to 20 mg.

55. The first and second antibody that binds DR5, or a pharmaceutically acceptable salt thereof, for use according to any one of claims 29 to 54, wherein the steroid hormone is administered at a dose of 10 mg.

56. The first and second antibody that binds DR5, or a pharmaceutically acceptable salt thereof, for use according to any one of claims 29 to 55, wherein the steroid hormone is administered by intravenous infusion.


SEQUENCE LISTING (48 sequences)

SEQ ID NO: 1 - protein, 8 aa, Artificial Sequence
  humanized VH region CDR1
GFNIKDTF

SEQ ID NO: 2 - protein, 8 aa, Artificial Sequence
  humanized VH region CDR2
IDPANGNT

SEQ ID NO: 3 - protein, 11 aa, Artificial Sequence
  humanized VH region CDR3
VRGLYTYYFDY

SEQ ID NO: 4 - protein, 118 aa, Artificial Sequence
  humanized VH region
EVQLQQSGAEVVKPGASVKLSCKASGFNIKDTFIHWVKQAPGQGLEWIGRIDPANGNTKYDPKFQGKATITTDTSSNTAYMELSSLRSEDTAVYYCVRGLYTYYFDYWGQGTLVTVSS

SEQ ID NO: 5 - protein, 6 aa, Artificial Sequence
  humanized VL region CDR1
QSISNN

SEQ ID NO: 6 - protein, 9 aa, Artificial Sequence
  humanized VL region CDR3
QQGNSWPYT

SEQ ID NO: 7 - protein, 107 aa, Artificial Sequence
  humanized VL region
EIVMTQSPATLSVSPGERATLSCRASQSISNNLHWYQQKPGQAPRLLIKFASQSITGIPARFSGSGSGTEFTLTISSLQSEDFAVYYCQQGNSWPYTFGQGTKLEIK

SEQ ID NO: 8 - protein, 8 aa, Artificial Sequence
  humanized VH region CDR2
IDPANTNT

SEQ ID NO: 9 - protein, 118 aa, Artificial Sequence
  humanized VH region
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSS

SEQ ID NO: 10 - protein, 8 aa, Artificial Sequence
  humanized VH region CDR1
GFNIKDTH

SEQ ID NO: 11 - protein, 11 aa, Artificial Sequence
  humanized VH region CDR3
ARWGTNVYFAY

SEQ ID NO: 12 - protein, 118 aa, Artificial Sequence
  humanized VH region
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSS

SEQ ID NO: 13 - protein, 5 aa, Artificial Sequence
  humanized VH region CDR1
SSVSY

SEQ ID NO: 14 - protein, 9 aa, Artificial Sequence
  humanized VH region CDR3
QQYHSYPPT

SEQ ID NO: 15 - protein, 106 aa, Artificial Sequence
  humanized VL region
DIQLTQSPSSLSASVGDRVTITCSASSSVSYMYWYQQKPGKAPKPWIYRTSNLASGVPSRFSGSGSGTDFTLTISSLQPEDFATYYCQQYHSYPPTFGGGTKVEIK

SEQ ID NO: 16 - protein, 10 aa, Artificial sequence
  VH region CDR1
GGSISSGDYF

SEQ ID NO: 17 - protein, 7 aa, Artificial Sequence
  VH region CDR2
IHNSGTT

SEQ ID NO: 18 - protein, 14 aa, Artificial Sequence
  VH region CDR3
ARDRGGDYYYGMDV

SEQ ID NO: 19 - protein, 122 aa, Artificial Sequence
  VH region
QVQLQESGPGLVKPSQTLSLTCTVSGGSISSGDYFWSWIRQLPGKGLECIGHIHNSGTTYYNPSLKSRVTISVDTSKKQFSLRLSSVTAADTAVYYCARDRGGDYYYGMDVWGQGTTVTVSS

SEQ ID NO: 20 - protein, 122 aa, Artificial Sequence
  VH region
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSS

SEQ ID NO: 21 - protein, 7 aa, Artificial Sequence
  humanized VH region CDR1
QGISRSY

SEQ ID NO: 22 - protein, 9 aa, Artificial Sequence
  humanized VH region CDR3
QQFGSSPWT

SEQ ID NO: 23 - protein, 108 aa, Artificial Sequence
  humanized VH region
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 24 - protein, 440 aa, Homo Sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 25 - protein, 448 aa, Artificial Sequence
  humanized heavy chain
EVQLQQSGAEVVKPGASVKLSCKASGFNIKDTFIHWVKQAPGQGLEWIGRIDPANGNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 448 aa, Artificial Sequence
  humanized heavy chain
EVQLQQSGAEVVKPGASVKLSCKASGFNIKDTFIHWVKQAPGQGLEWIGRIDPANGNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHGALHNHYTQKSLSLSPGK

SEQ ID NO: 27 - protein, 214 aa, Artificial Sequence
  humanized light chain
EIVMTQSPATLSVSPGERATLSCRASQSISNNLHWYQQKPGQAPRLLIKFASQSITGIPARFSGSGSGTEFTLTISSLQSEDFAVYYCQQGNSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 448 aa, Artificial Sequence
  humanized heavy chain
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 29 - protein, 448 aa, Artificial Sequence
  humanized heavy chain
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 448 aa, Artificial Sequence
  humanized heavy chain
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 31 - protein, 448 aa, Artificial Sequence
  humanized heavy chain
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 448 aa, Artificial Sequence
  humanized heavy chain
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 33 - protein, 448 aa, Artificial Sequence
  humanized heavy chain
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 34 - protein, 448 aa, Artificial Sequence
  humanized heavy chain
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHGALHNHYTQKSLSLSPGK

SEQ ID NO: 35 - protein, 213 aa, Artificial Sequence
  humanized light chain
DIQLTQSPSSLSASVGDRVTITCSASSSVSYMYWYQQKPGKAPKPWIYRTSNLASGVPSRFSGSGSGTDFTLTISSLQPEDFATYYCQQYHSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 411 aa, Artificial Sequence
  modified human DR5
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLANQKIEDHLLSSGKFMYLEGNADSAMS

SEQ ID NO: 37 - protein, 411 aa, Homo Sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 38 - protein, 440 aa, Homo Sapiens [sequence omitted]

SEQ ID NO: 39 - protein, 445 aa, Macaca fascicularis
MGQLRQSAPAASGARKGRGPGPREARGARPGLRVLKTLVLVVAAARVLLSVSADCAPITRQSLDPQRRAAPQQKRSSPTEGLCPPGHHISEDSRECISCKYGQDYSTHWNDFLFCLRCTKCDSGEVEVNSCTTTRNTVCQCEEGTFREEDSPEICRKCRTGCPRGMVKVKDCTPWSDIECVHKESGTKHTGEVPAVEKTVTTSPGTPASPCSLSGIIIGVIVLVVIVVVAVIVWKTSLWKKVLPYLKGVCSGGGGDPERVDSSSHSPQRPGAEDNALNEIVSIVQPSQVPEQEMEVQEPAEQTDVNTLSPGESEHLLEPAKAEGPQRRGQLVPVNENDPTETLRQCFDDFAAIVPFDAWEPLVRQLGLTNNEIKVAKAEAASSRDTLYVMLIKWVNKTGRAASVNTLLDALETLEERLAKQKIQDRLLSSGKFMYLEDNADSATS

SEQ ID NO: 40 - protein, 416 aa, Artificial sequence
  modified Macaca fascicularis
MGQLRQSAPAASGARKGRGPGPREARGARPGLRVLKTLVLVVAAARVLLSVSADCAPITRQSLDPQRRAAPQQKRSSPTEGLCPPGHHISEDSRECISCKYGQDYSTHWNDFLFCLRCTKCDSGEVEVNSCTTTRNTVCQCEEGTFREEDSPEICRKCRTGCPRGMVKVKDCTPWSDIECVHKESGIIIGVIVLVVIVVVAVIVWKTSLWKKVLPYLKGVCSGGGGDPERVDSSSHSPQRPGAEDNALNEIVSIVQPSQVPEQEMEVQEPAEQTDVNTLSPGESEHLLEPAKAEGPQRRGQLVPVNENDPTETLRQCFDDFAAIVPFDAWEPLVRQLGLTNNEIKVAKAEAASSRDTLYVMLIKWVNKTGRAASVNTLLDALETLEERLANQKIQDRLLSSGKFMYLEDNADSATS

SEQ ID NO: 41 - protein, 381 aa, Mus Musculus
MEPPGPSTPTASAAARADHYTPGLRPLPKRRLLYSFALLLAVLQAVFVPVTANPAHNRPAGLQRPEESPSRGPCLAGQYLSEGNCKPCREGIDYTSHSNHSLDSCILCTVCKEDKVVETRCNITTNTVCRCKPGTFEDKDSPEICQSCSNCTDGEEELTSCTPRENRKCVSKTAWASWHKLGLWIGLLVPVVLLIGALLVWKTGAWRQWLLCIKRGCERDPESANSVHSSLLDRQTSSTTNDSNHNTEPGKTQKTGKKLLVPVNGNDSADDLKFIFEYCSDIVPFDSWNRLMRQLGLTDNQIQMVKAETLVTREALYQMLLKWRHQTGRSASINHLLDALEAVEERDAMEKIEDYAVKSGRFTYQNAAAQPETGPGGSQCV

SEQ ID NO: 42 - protein, 450 aa, Artificial Sequence
  modified human DR5
MEQRGQNAPAASGARKRHGPGPREARGARPGLRVPKTLVLVVAAVLLLVSAESALITQQDLAPQQRVAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEAPAVEETVTSSPGTPASPCSAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGKHHHHHHHHEPEA

SEQ ID NO: 43 - protein, 423 aa, Artificial Sequence
  modified human DR5
MEQRGQNAPAASGARKRHGPGPREARGARPGPRVPKTLVLVVAAVLLLVSAESALITQQDLAPQQRAAPQQKRSSPSEGPCLAGQYLSEGNCKPCREGIDYTSHSNHSLDSCILCTRCDSGEVELSPCTTTRNTVCQCEEGTFREEDSPEMCRKCRTGCPRGMVKVGDCTPWSDIECVHKESGAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGKHHHHHHHHEPEA

SEQ ID NO: 44 - protein, 423 aa, Artificial Sequence
  modified human DR5
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCKPGTFEDKDSPEICQSCSNCTDGEEEVGDCTPWSDIECVHKESGAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGKHHHHHHHHEPEA

SEQ ID NO: 45 - protein, 154 aa, Artificial Sequence
  modified human DR5
MGWSCIILFLVATATGVHSQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGHHHHHHHH

SEQ ID NO: 46 - protein, 330 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 447 aa, Artificial Sequence
  humanized heavy chain
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 48 - protein, 447 aa, Artificial Sequence
  humanized heavy chain
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPG